(19) United States
(12) Patent Application Publication
(10) Pub. No.: US 2015/0191446 A1
(43) Pub. Date: Jul. 9, 2015

(54) SELENO-COMPOUNDS AND THERAPEUTIC USES THEREOF

(71) Applicants: THE HEART RESEARCH INSTITUTE LTD, Newtown (AU); THE UNIVERSITY OF MELBOURNE, Carlton (AU)

(72) Inventors: Carl Herbert SCHIESSER, Carlton (AU); Corin STORKEY, Carlton (AU); Michael Jonathan DAVIES, Newtown (AU)

(73) Assignees: THE HEART RESEARCH INSTITUTE LTD, Newtown (AU); THE UNIVERSITY OF MELBOURNE, Carlton (AU)

(21) Appl. No.: 14/512,193

(22) Filed: Oct. 10, 2014

Related U.S. Application Data
(63) Continuation-in-part of application No. 13/881,594, filed on Feb. 27, 2014.

(30) Foreign Application Priority Data
Oct. 28, 2010 (AU) 2010904801

(51) Int. Cl. C07D 345/00

(52) U.S. Cl. CPC C07D 345/00

(57) ABSTRACT

Compounds and compositions, useful as antioxidants and in particular to selenium containing compounds of formula (I): wherein n is 1, 2, or 3; m is 2, 3, 4, or 5; and each R] is independently-(optionally substituted C1-C3 alkylene)p-OH, where p is 0 or 1, or a salt thereof. These seleno-compounds may be used in the treatment of diseases or conditions associated with increased levels of oxidants produced by myeloperoxidase (MPA), such as for instance, atherosclerosis and diabetes.

This application is a Continuation in Part of U.S. application Ser. No. 13/881,594, filed Feb. 7, 2014, which is a §371 National Stage Application of International Application No. PCT/AU11/01391, filed Oct. 28, 2011, which claims priority to Australian Application No. 2010904801, filed Oct. 28, 2010, the disclosure of each is incorporated herein by reference.

The patent or application file contains at least one drawing executed in color. Copies of this patent or patent application publication with color drawing(s) will be provided by the Office upon request and payment of the necessary fee.

This Substitute Specification contains no new matter.

### FIELD

The present invention relates to compounds and compositions useful as antioxidants and in particular to selenium containing compounds (“seleno-compounds”). The invention also relates to the use of these seleno-compounds and compositions comprising at least one seleno-compound in the treatment of diseases or conditions associated with increased levels of oxidants produced by myeloperoxidase (MPO), such as for instance, atherosclerosis.

The invention also has particular application to the use of these seleno-compounds comprising at least one seleno-compound in the treatment of diabetic wounds, particularly chronic wounds in patients suffering diabetes.

### BACKGROUND

Myeloperoxidase (MPO) is a mammalian enzyme that is released at sites of inflammation from intracellular granules by activated neutrophils, monocytes and some macrophages (S. J. Klebanoff, Proc. Assoc. Am. Physicians, 1999, 111:383-389). The activation of these cells also results in the production of hydrogen peroxide (H₂O₂) by NADPH oxidase enzymes via a respiratory burst (B. M. Babior, Trends Biochem. Sci., 1987, 12:241-243); MPO utilizes H₂O₂ to oxidize halide and pseudo-halide ions, predominantly chloride (C⁻), bromide (Br⁻) and thiocyanate (SCN⁻), to generate the potent oxidants, hypochlorous (HOCl), hypobromous (HOBr) and hypothiocyanous acid (HOSCN), respectively (see FIG. 1). The proportions of each of these reactive species present in human plasma is determined by the selectivity constants of MPO for each ion respectively. Therefore at neutral pH and normal physiological plasma concentrations approximately 45% of the hydrogen peroxide consumed by MPO results in the formation of HOCl, 50% HOSCN, with the remaining 5% yielding HOBr (C. J. van Dalen, M. W. Whitehouse, C. C. Winterbourn and A. J. Kettle, Biochem. J., 1997, 327:487-492).

These hypohalous acids (HOX) are key components of the inflammatory response and are bactericidal but have also been linked to several human pathologies as a result of damage to host tissue. Overproduction of HOX may contribute to the oxidative stress associated with diseases such as atherosclerosis and diabetes. The evidence for a role of MPO and its oxidants in the pathogenesis of atherosclerosis is particularly compelling (A. Hoy, B. Leininger-Muller, D. Kutter, G. Siest and S. Visvikis, Clin. Chem. Lab. Med., 2002, 40:2-8; R. Stocker and J. F. Keaney, Jr., Physiol. Rev., 2004, 84:1381-1478; and E. Malle, G. Marsche, J. Arnhold and M. J. Davies, Biochem. Biophys. Acta., 2006, 1761:392-415), but strong evidence exists that these oxidants are also involved in other diseases such as cystic fibrosis, sepsis, rheumatoid arthritis, some cancers, asthma, and kidney disease, amongst others (A. Hoy, B. Leininger-Muller, D. Kutter, G. Siest and S. Visvikis, Clin. Chem. Lab. Med., 2002, 40:2-8; R. Zhang, M.-L. Brennan, X. Fu, R. J. Aviles, G. L. Pearce, M. S. Penn, E. J. Topol, D. L. Sprecher and S. L. Hazen, J. Am. Med. Assoc., 2001, 286:2136-2142; H. Ohshima, M. Tatemichi and T. Sawa, Arch. Biochem. Biophys., 2003, 417:3-11; E. A. Podrez, H. M. Abu-Soud and S. L. Hazen, Free Radical Bioi. Med., 2000, 28:1717-1725; F. J. Kelly and I. S. Mudway, Amino Acids, 2003, 25:375-396; E. Malle, T. Buch and H.-J. Grone, Kidney Int., 2003, 64:1956-1967; A. VanDer Vliet, M. N. Nguyen, M. K. Shigenaga, J. P. Eiserich, G. P. Marelich and C. E. Cross, Am. J. Physiol. Lung Cell Mol. Physiol., 2000, 279, L537-546; and J. M. S. Davies, D. A. Horwitz and K. J. A. Davies, Free Radical Biol. Med., 1993, 15:637-643). Clinical studies have shown that elevated plasma MPO levels are a strong independent risk factor, and predictor of outcomes, for cardiovascular disease (R. Zhang, M.-L. Brennan, X. Fu, R. J. Aviles, G. L. Pearce, M. S. Penn, E. J. Topol, D. L. Sprecher and S. L. Hazen, J. Am. Med. Assoc., 200 I, 286:2136-2142). Studies have also shown a direct link between HOCL-mediated protein damage and atherosclerosis with MPO protein and chlorinated residues of the amino acid Tyrosine being detected in atherosclerotic lesions and the latter identified as a specific marker for HOCl-mediated protein oxidation (L. J. Hazell, G. Baernthaler and R. Stocker, Free Radical Biol. Med., 2001, 31:1254-1262; and S. L. Hazen and J. W. Heinecke, J Clin. Invest., 1997, 99:2075-2081). Through in vitro model studies it has been shown that plasma proteins consume the majority of HOCl with limited damage to other materials. Protein oxidation by HOCl has been studied in detail with the amino acids Met, Cys, Trp, Tyr, Lys, and His established as the major targets (M. J. Davies, C. L. Hawkins, D. I. Pattison and M. D. Rees, Antioxid. Redox Signaling, 2008, 10:1199-1234).

Accordingly, it would be advantageous to identify and develop classes of therapeutic compounds which could regulate the presence of reactive oxygen species (ROS), such as hypohalous acids (e.g., HOCl and HOBr) and/or to minimise the adverse impact of such ROS by inhibiting or minimising the pathogenesis of certain conditions or disease states which are linked to tissue damage by ROS.

### SUMMARY OF THE INVENTION

The present invention thus provides a class of seleno-compounds which possess the ability to protect tissue (and specifically proteins) from ROS mediated damage. More specifically the present invention provides compounds which comprise a stable seleno-moiety, which acts as a radical scavenger and in particular a scavenger of ROS or free-radicals derived from non-radical ROS and as such is able to function as antioxidants.

The invention is based on the discovery that certain seleno-compounds display unique properties including antioxidant activity and aqueous solubility (and plasma solubility).

Accordingly, the seleno-compounds of the present invention may function as effective agents for treating diseases and conditions, which are linked to the production of and damage by free-radicals derived from ROS. Such compounds have significant potential in treating, for instance, atherosclerosis, cystic fibrosis, sepsis, rheumatoid arthritis and other inflammatory disorders, some cancers, asthma, and cardiovascular diseases.

In an aspect the invention provides compounds of formula (1):

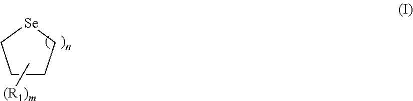

wherein
n is l, 2, or 3;
m is 2, 3, 4; or 5; and
each R₁ is independently-(optionally substituted C₁-C₃ alkylene)ₚ-OH, where p is 0 or 1.

In a further aspect of the invention there is provided a method for the treatment of oxidative stress comprising the administration of a seleno-compound of formula (I), or a pharmaceutically acceptable salt thereof, or a composition comprising a seleno-compound of formula (I), or a pharmaceutically acceptable salt thereof.

In another aspect the invention provides the use of a seleno-compound of formula (I), or a salt thereof, in the manufacture of a medicament for the treatment of oxidative stress.

In a further aspect the invention provides the use of a seleno-compound of formula (I), or a salt thereof, for the treatment of oxidative stress.

In a preferred aspect the oxidative stress is associated with a disease. The disease may be atherosclerosis, cystic fibrosis, sepsis, rheumatoid arthritis and other inflammatory disorders, some cancers, asthma, and cardiovascular diseases. In another preferred aspect the oxidative stress is associated with non-healing chronic wounds, particularly wounds associated with diabetes. Furthermore the disease may be associated with a human or an animal.

In a further preferred aspect the disease is atherosclerosis.

In a further aspect the invention provides a method of protecting against chloramine formation by HOCl, said method comprising the step of administering to a subject a compound of formula (I).

In a further aspect the invention provides a method of protecting a protein from HOCl and HOBr-mediated oxidation said method comprising the step of contacting said protein with a compound of formula (I).

In another aspect of the invention there is provided a method of scavenging free-radicals said method comprising the steps of contacting a source of said free-radicals with a seleno-compound of formula (I), or a pharmaceutically acceptable salt thereof for a time and under suitable conditions.

The above three methods may be conducted both in vivo and ex vivo. The in vivo method would involve treating (i.e., administering) a subject in need thereof with a seleno-compound of the invention.

In a further aspect of the invention there is provided a pharmaceutical composition for use as an antioxidant, the composition comprising an effective amount of a seleno-compound of formula (I), or a pharmaceutically acceptable salt thereof and optionally a carrier or diluent.

In another aspect of the invention there is provided novel processes for the preparation of seleno-compounds of formula (I).

### BRIEF DESCRIPTION OF THE DRAWINGS

FIG. 1 is a schematic diagram summarising the reactions involved in myeloperoxidase (MPO) production of halogenated oxidants, and their subsequent reactions. It should be noted that both MPO can also generate other oxidants from additional anions (e.g. HOSCN from SCN⁻, NO₂ from NO₂⁻).

FIG. 2A is an HPLC electrochemical (EC) trace for NAc-Tyrosine. Lowest concentration of seleno-sugar (light brown, Se 10h)—Highest concentration of seleno-sugar (dark green, Se5h). 2B is an HPLC fluorescence trace (λₑₓ, 265 nm; λₑₘ, 310 nm) for FMoc-methionine sulfoxide. Lowest concentration of seleno-sugar (pink, Se0)—Highest concentration of seleno-sugar (dark green, Se8).

FIGS. 3A and 3B are graphs of ([Substrate].Yₘₐₓ/Y_{quench} mM) as a function of Se-sugar concentration (μM) for determination of HOBr and HOCl scavenging rates. (Errors expressed as 95% confidence intervals (=standard error X t₉₅).

k₂(HOBr+substrate)=gradient×k₂(HOBr+NAc−Tyr):k₂(HOBr+NAc−Tyr)=2.610⁵M⁻¹s⁻¹).

k₂(HOCl+substrate)=gradient×k₂(HOCl+FMocMet):k₂(HOCl+FMocMet)=1.310⁸M⁻¹s⁻¹).

FIG. 3C is a table summarizing the calculated rate constants for each seleno-sugar against HOBr and HOCl

FIGS. 4A to 4J are bar graphs depicting the protection of amino acids from HOCl-mediated oxidation in BSA or human plasma by various seleno-sugars. N=4, with error bars representing standard error of mean. *=p<0.05, assessed by 2-tailed 1-way ANOVA (with Tukey's post-hoc test) relative to control (i.e. demonstrating significant damage vs. control) or #=p<0.05 relative to 0 mM (i.e. demonstrating significant protection vs. 0 mM).

FIG. 5A is a bar graph which shows the seleno-gulose derivative (SeGul, compound 4) effectively preventing 3-chloro-tyrosine formation by HOCl in BSA. FIG. 5B is a bar graph which shows the seleno-gulose derivative effectively preventing 3-chloro-tyrosine formation by HOCl in human plasma. N=3, with error bars representing standard error of mean. #=p<0.05 relative to 0 mM assessed by 2-tailed 1-way ANOVA relative to 0 mM (i.e. demonstrating significant protection vs. 0 mM).

FIG. 6A to 6E are graphs depicting the percentage of chlorinated taurine (FIG. 6A), lysine (FIG. 6B), glycine (FIG. 6C), histidine (FIG. 6D), and bovine serum albumin (BSA, FIG. 6E) remaining after treatment with increasing concentrations of different compounds. Data represents mean±SD, n=3. Compounds are selenomethionine (SeMet), Se-methylselenocysteine (MeSeSys), diselenosystamine (SeCysta), methionine (Met), cysteine (Cys) and the seleno compounds of the invention SeTal (compound 38) and 6-SeGul (compound 4).

FIG. 6F is a table showing IC₅₀ values determined by TMB assay for scavenging of chloramines, calculated from the data displayed in FIGS. 6A-6E using the log [inhibitor] vs. normalized response function as calculated by the software program Prism 5.0.

FIGS. 7A and 7B are graphs depicting the percentage thiol remaining after treatment of glutathione (GSH), cysteine (Cys)•and bovine serum albumin (BSA) with selenomethionine oxide (SeMetO) (FIG. 7A) and the seleno compound of the invention SeTal oxide.

FIG. 8 depicts the protocol for cell survival experiments (cytotoxic effects of the seleno-compounds of the invention). Cells were incubated with the test compound for 24 or 48 h and then MTT for 2 h. MTT is converted by living cells into a purple formazan. This is solubilised with DMSO and quantified by measuring the absorbance of each well at 595 nm λ.

FIGS. 9A, 9B and 9C depicts the effects of seleno-compounds (1 mM) or staurosporine (0.01-1 μM) on CHO or glial cell survival. Cells were pre-incubated with compounds 24 or 48 h at 37° C. and cell survival detected using MTT (2 mg/ml). Data is expressed as (mean±SEM) absorbance values expressed as a percentage of control (100%, cells treated with PBS only). *P<0.05 vs. 100% (dashed line); one-sample t test. n values refer to experiments conducted on different cell passages or taken from separate animals for CHO or glial cells, respectively.

FIG. 10 illustrates wound closure observed in the db/db mouse models topically treated with a 1 mM saline solution of 1,4-anhydro-4-seleno-D-talitol (selenosugar) (b) and a non-selenium control sugar, 1,4-anydro-D-talitol (a).

FIG. 11 illustrates vascular perfusion measured by Doppler imaging and corresponding to the selenosugar treated wound (1) and the untreated control wound (2) of the db⁻/db⁻ mouse model of FIG. 1 (where * indicates p<0.05, and n=6). Doppler imaging is a non-invasive technique used to evaluate blood flow velocity with red blood cells as the moving target;

FIGS. 12A to 21B illustrate comparative tissue histology for (a) the wildtype and (b) db/db mouse models, both (i) non-treated mice, (ii) mice treated for 5 days and (iii) mice treated for 10 days. The figures also illustrate plots against time in days for the following wound parameters for (i), (ii) and (iii) (where * indicates p<0.05, ** indicates p<0.01, *** indicates p<0.001);

FIGS. 12A and 12B relate to % neutrophils;

FIGS. 13A and 13B relate to % interlukin-6 (IL-6);

FIGS. 14A and 14B relate to % actin;

FIGS. 15A and 15-B relate to % elastin;

FIGS. 16A and 16-B relate to % caspase 3 protein;

FIGS. 17A and 17-B relate to % F4/80 (a mouse macrophage-specific membrane marker);

FIGS. 18A and 18-B relate to % monocyte chemotactic protein-1 (MCP-1);

FIGS. 19A and 19-B relate to % myeloperoxidase (MPO);

FIGS. 20A and 20-B relate to % vascular endothelial growth factor (VEGF);

FIGS. 21A and 21-B relates to % von Willebrand factor (vWF) a blood glycoprotein involved in halting the escape of blood from vessels following vascular injury;

FIGS. 22A and 22-B provide a plot illustrating performance of a selenosugar according to the present invention (1,4-anhydro-4-seleno-D-talitol, 1 mM) (3) against performance of a control sugar of identical structure in which the Se is replaced with oxygen (1,4-anydro-D-talitol) (4) in wildtype mice (FIG. 13a) and in db/db knockout mice (FIG. 13(b)) where *** indicates p<0.001 and **** indicates p<0.0001 and N=6;

FIG. 23 is a plot illustrating the performance of a selenosugar according to the present invention (1,4-anhydro-4-seleno-D-talitol) (5), against a known antioxidant (6) with similar in vitro antioxidant capacity (selenomethionine) at the same dose in wildtype mice where ** indicates p<0.01 and **** indicates p, 0.0001 and N=5;

FIGS. 24A and 24B provide a plot illustrating the performance of a selenosugar according to the present invention (1,4-anhydro-4-seleno-D-talitol) (8) against a similar water-soluble selenium compound that is not sugar derived (DHS_{red}) (10) in non-diabetic (wt) mice over time. FIG. 15a illustrates % wound closure in terms of the wound size and FIG. 15b illustrates blood flow intensity (Doppler measurement) in arbitrary units where ** indicates p<0.01, *** P<0.001 and **** p<0.0001 and N=6;

FIG. 25 illustrates a wound treated with a selenosugar according to the present invention (ii) and a wound treated with DHS_{red} (i) at the same dose. Note the development of pus in wound treated with DHS_{red} and the inferior healing;

FIG. 26 illustrates typical differences in scar tissue that forms during wound healing in wildtype mice having a wound treated with a selenosugar according to the present invention (ii) and a wound treated with DHS_{red} (i) at the same dose. Note the pronounced scar tissue build up in the wound treated with DHS_{red}, while the wound treated with the selenosugar has significantly less scar tissue and is better healed.

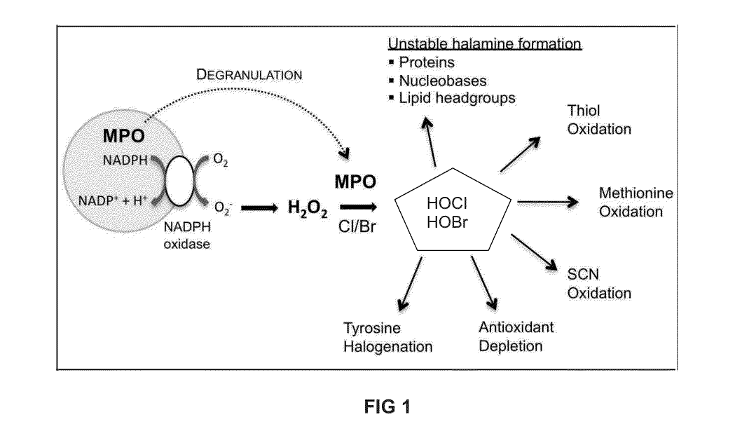

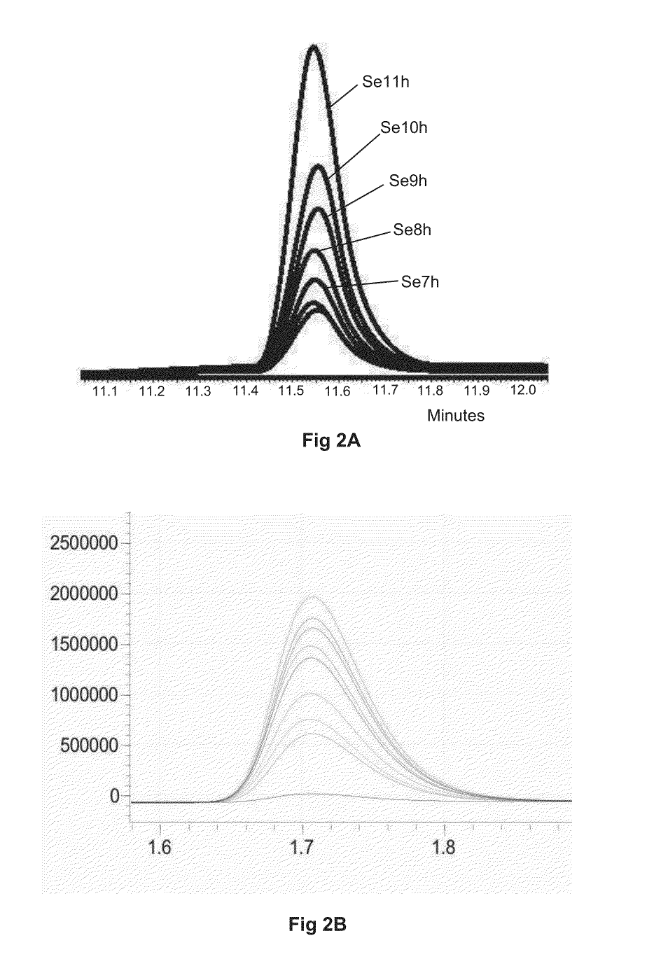

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “oxidative stress” refers to an abnormal level of reactive oxygen species (ROS). Oxidative stress may be induced. by, for example an increase in the levels of free radicals such as hydroxyl, nitric acid or superoxide or an increase in the levels of non-radicals such as hydrogen peroxide, lipid peroxide and hypohalous acid which may themselves be a source of free-radicals. Increased ROS levels may occur as a result of a number of activities or conditions including infections, inflammation, ageing, UV radiation, pollution, excessive alcohol consumption, and cigarette smoking. Oxidative stress may lead to oxidative damage of particular molecules such as proteins and lipids with consequential injury to cells, tissues or organs. Thus, oxidative stress is involved in a number of diseases including cancer, ischemia-reperfusion injury, infectious disease, inflammatory disease, autoimmune diseases, cardiovascular diseases. For a review on oxidative stress and related conditions/diseases the reader is referred to J. Ocul. Pharmacal Ther. 2000 April; 16(2):193-201 which is incorporated herein by reference.

For example, LDL (low density lipoprotein) may become oxidised during periods of oxidative stress and induce the formation of macrophage-derived foam cells. These foam cells are present in pre-atherosclerotic fatty-streak lesions and advanced atherosclerotic plaques. This link between oxidative stress and atherosclerosis is supported by findings that the antihyperlipidemic drug probucol exhibits an antioxidative activity and is effective for the treatment of arterial sclerosis.

In addition, the heme enzyme myeloperoxidase (MPO) is released at sites of inflammation by activated leukocytes. A key function of MPO is the production of hypohalous acids (HOX, X=Cl, Br), which are strong oxidants with potent antibacterial properties. However, HOX can also damage host tissue when produced at the wrong place, time or concentration; this has been implicated in several human diseases (e.g. atherosclerosis, some cancers). Thus, elevated blood and leukocyte levels of MPO are significant independent risk factors for atherosclerosis, while specific markers of HOX-mediated protein oxidation are often present at elevated levels in patients with HOX. Reaction with amines generates chloramines (RNHCl) and bromamines (RNHBr), which are more selective oxidants than HOX and are key intermediates in HOX biochemistry. These species are known to be formed in high yield on a range of protein targets, including proteins in human plasma, on exposure to HOCl. As such it is important to develop therapeutic compounds that can also scavenge these materials in a rapid and effective manner.

“Alkylene” refers to a divalent alkyl group. Examples of such alkylene groups include methylene (—CH₂—), ethylene (—CH₂CH₂—), and the propylene isomers (e.g., —CH₂CH₂CH₂— and —CH(CH₃)CH₂—).

“Optionally substituted” in the context of the present invention is taken to mean that a hydrogen atom on the alkylene chain may be replaced with a group selected from hydroxyl, amino, or thio. More preferably the substituent is hydroxyl.

In a preferred aspect the present invention provides stable, aqueous soluble 5, 6 and 7 membered selenocycles of formula (I) wherein the compound is not metabolisable or derivatisable (to any great extent) by the body. In this regard as there are no known mammalian enzymes that process L-sugars, in particular L-gulose and L-idose, in a further preferred aspect the seleno-cycles of formula (I) are seleno-derivatives of L-sugars.

In an embodiment n is 1.

In an embodiment n is 2.

In an embodiment n is 3.

In an embodiment n is 1 and m is 2, 3, or 4.

In an embodiment n is 2 and m is 2, 3, 4, or 5.

In an embodiment n is 3 and m is 2, 3, 4, and 5:

In an embodiment n is 1 or 2 and m is 2, 3, or 4.

In an embodiment n is 1 or 2, m is 2, 3, or 4 and at least one R₁ is (optionally-substituted (C₁-C₃)alkylene)ₚ-OH where p=1.

In an embodiment n is 1 or 2, m is 2, 3, or 4 and one R₁ is (optionally substituted C₁-C₃alkylene)ₚ-OH where p=1.

In an embodiment n is 2, m is 4, and one R₁ is (optionally substituted C₂-C₃alkylene)ₚ-OH where p=1.

In the above embodiments preferably the (optionally substituted C₁-C₃alkylene)ₚ-OH group is optionally substituted C₂-alkylene-OH or C₁-alkylene-OH. More preferably the group is —CH₂—OH.

In the above embodiments where the C₁-C₃ alkylene group is substituted it is substituted with a hydroxyl group, for example —CH(OH)—CH₂OH.

Examples of seleno-compounds of formula (I) include:

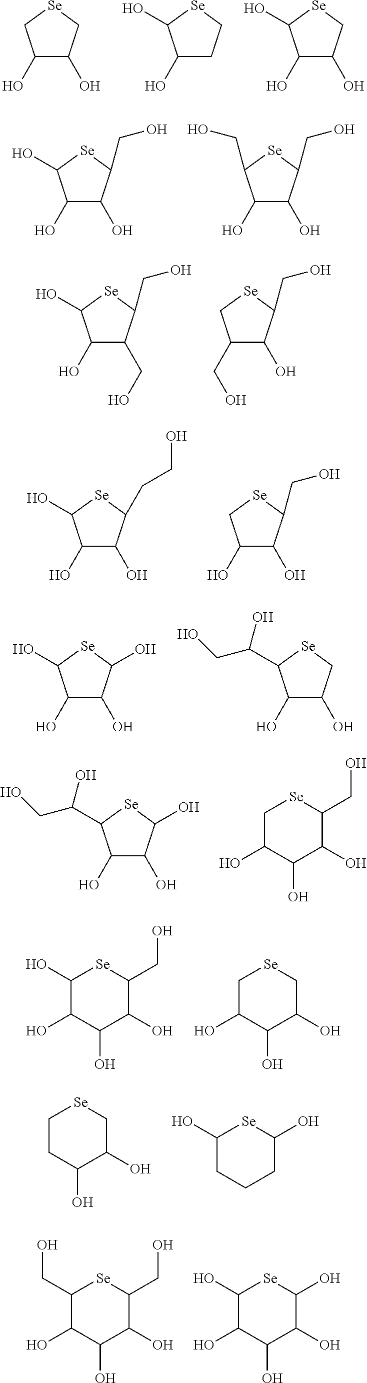

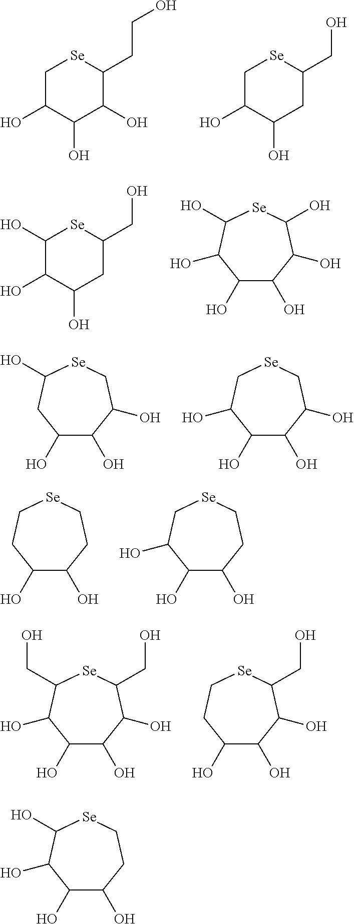

In an embodiment the seleno-compound of formula (I) is represented by

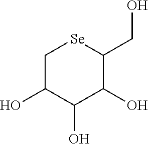 [SMILES: OCC1[Se]CC(O)C(O)C1O]

In a further embodiment the seleno-compound of formula (I) is represented by

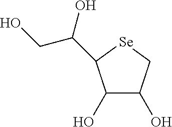 [SMILES: OCC(O)C1[Se]CC(O)C1O]

The compounds of the invention may be in crystalline form either as the free compounds or as solvates (e.g. hydrates) and it is intended that both forms are within the scope of the present invention. Methods of solvation are generally known within the art.

It will also be recognised that compounds of the invention may possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or mixtures may be resolved by conventional methods, e.g., chromatography, or use of a resolving agent.

Alternatively, enantiomerically pure seleno-compounds of formula (I) may be prepared from carbohydrates. In this regard preferred compounds of the present invention may be representative seleno-derivatives of known monosaccharides where the selenium is in. the ring position. Examples of suitable seleno-compounds of this sort may be derived from either D- or L-aldoses such as ribose, arabinose, xylose, lyxose, allose, altrose, glucose, mannose, gulose, idose, galactose, and talose. Preferably the seleno-compounds are derivatives of L-aldoses. Representative examples include:

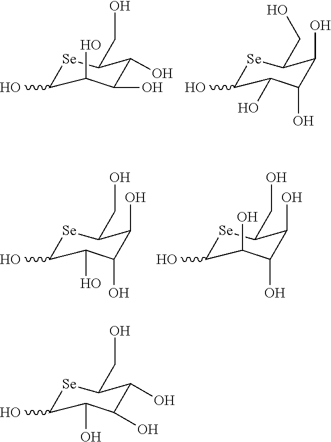

(shown as mixtures of α and β anomers)

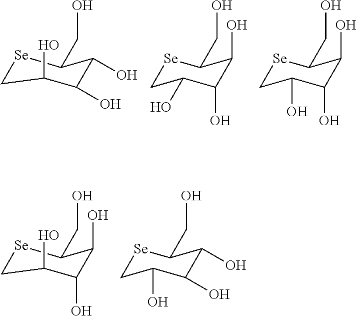

(representation of 1,5-anhydro series)

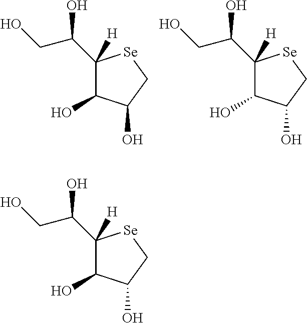 [SMILES: OC[C@@H](O)[C@@H]1[Se]C[C@@H](O)[C@H]1O ; OC[C@@H](O)[C@@H]1[Se]C[C@H](O)[C@@H]1O ; OC[C@@H](O)[C@@H]1[Se]C[C@H](O)[C@H]1O]

(example representation of 1,4-anhydro series)

In an embodiment the compound is selected from one of following:
1,5-anhydro-5-seleno-L-gulitol
1,5-anhydro-5-seleno-L-mannitol
1,5-anhydro-5-seleno-L-iditol
1,5-anhydro-5-seleno-L-glucitol
1,5-anhydro-5-seleno-L-galitol
1,5-anhydro-5-seleno-L-talitol
1,5-anhydro-5-seleno-L-allitol
1,5-anhydro-5-seleno-L-altritol

In an embodiment the compound is selected from one of the following:
1,5-anhydro-5-seleno-D-gulitol
1,5-anhydro-5-seleno-D-mannitol
1,5-anhydro-5-seleno-D-iditol
1,5-anhydro-5-seleno-D-glucitol
1,5-anhydro-5-seleno-D-galitol
1,5-anhydro-5-seleno-D-talitol
1,5-anhydro-5-seleno-D-allitol
1,5-anhydro-5-seleno-D-altritol

In an embodiment the compound is selected from one of the following:
1,4-anhydro-4-seleno-L-gulitol
1,4-anhydro-4-seleno-L-mannitol
1,4-anhydro-4-seleno-L-iditol
1,4-anhydro-4-seleno-L-glucitol
1,4-anhydro-4-seleno-L-galitol
1,4-anhydro-4-seleno-L-talitol
1,4-anhydro-4-seleno-L-allitol
1,4-anhydro-4-seleno-L-altritol

In another embodiment the compound is selected from one of the following:
1,4-anhydro-4-seleno-D-gulitol
1,4-anhydro-4-seleno-D-mannitol
1,4-anhydro-4-seleno-D-iditol
1,4-anhydro-4-seleno-D-glucitol
1,4-anhydro-4-seleno-D-galitol
1,4-anhydro-4-seleno-D-talitol
1,4-anhydro-4-seleno-D-allitol
1,4-anhydro-4-seleno-D-altritol

The seleno-compounds of the present invention can be prepared based on the modification of the synthetic procedures described in, for example, M. A. Lucas et al., Tetrahedron, 2000,56:3995-4000 and C. Storkey et al., Chem. Comm., 2011,47, 9693-9695.

In•respect of compounds of formula (I) some examples of suitable synthetic approaches are depicted in the below schemes.

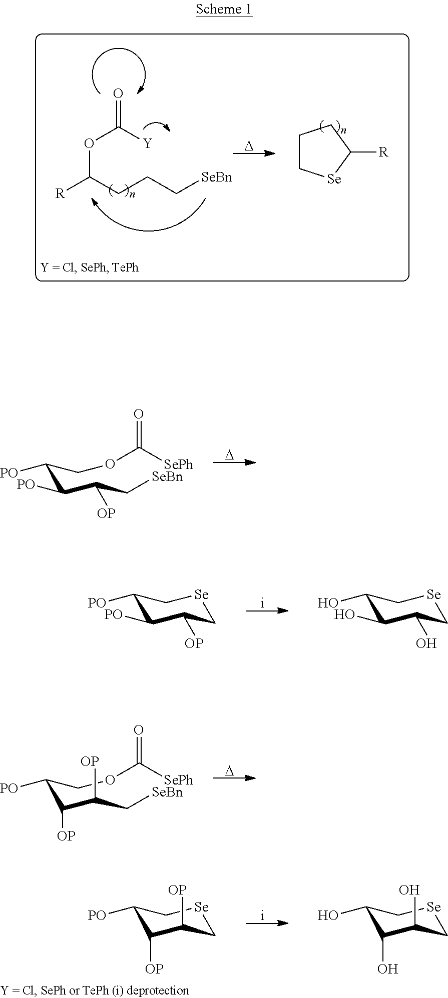

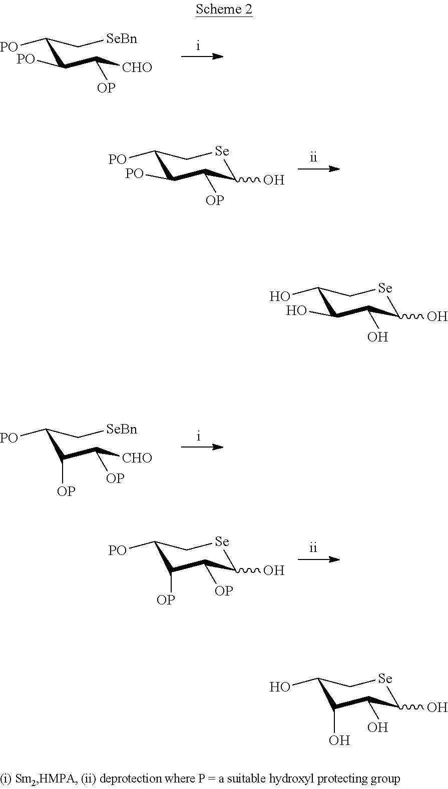

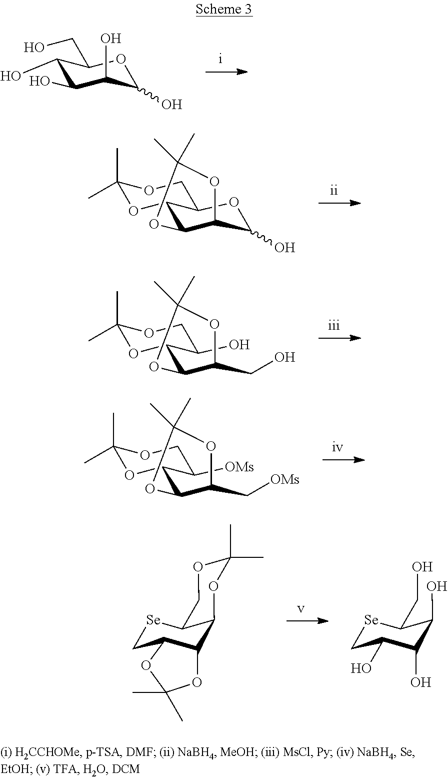

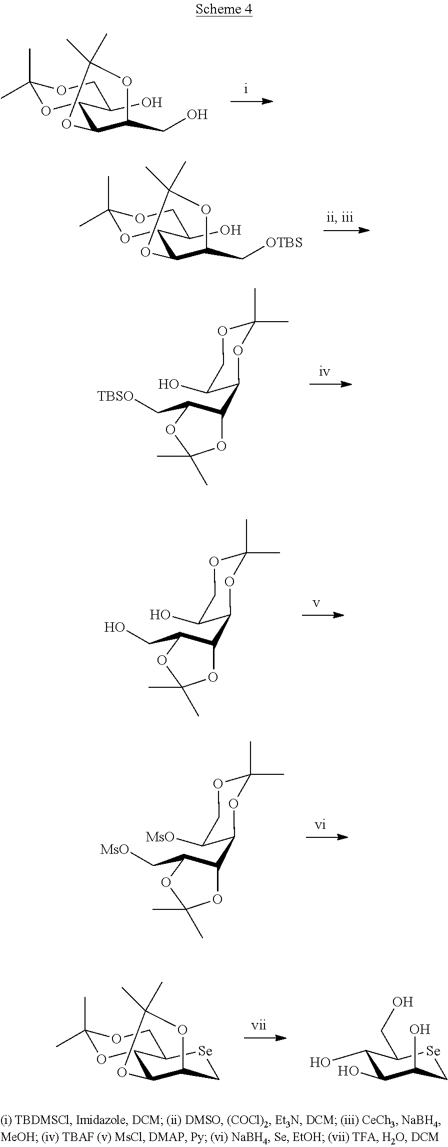

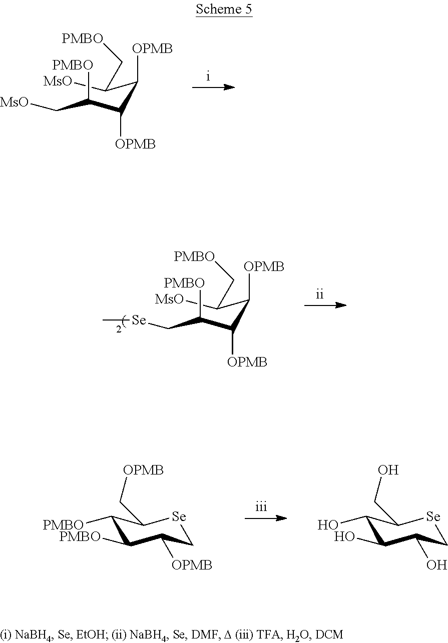

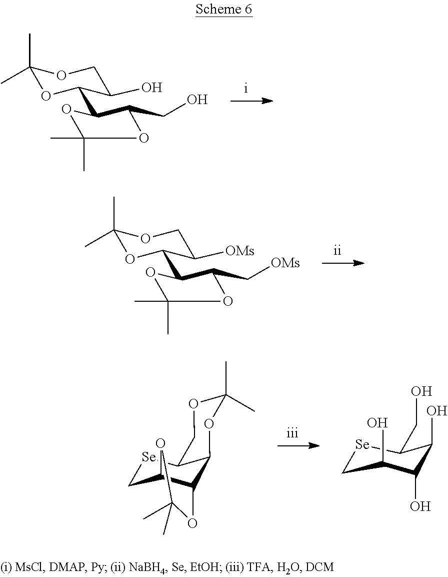

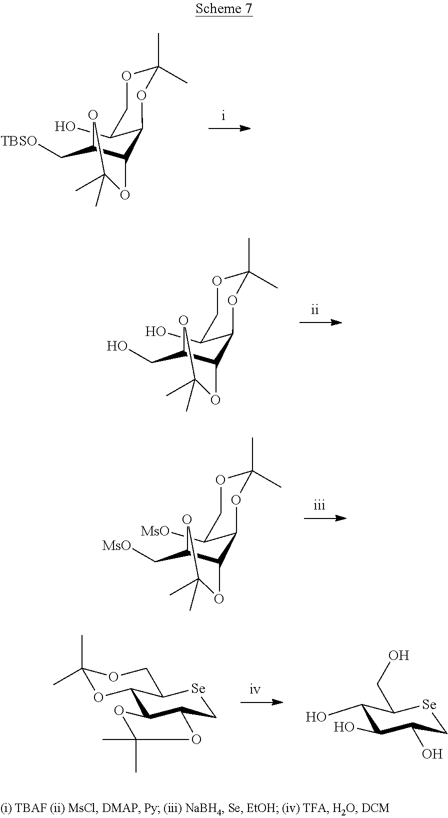

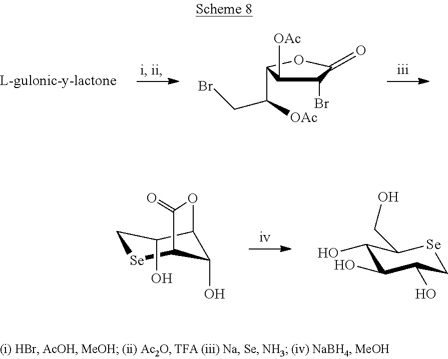

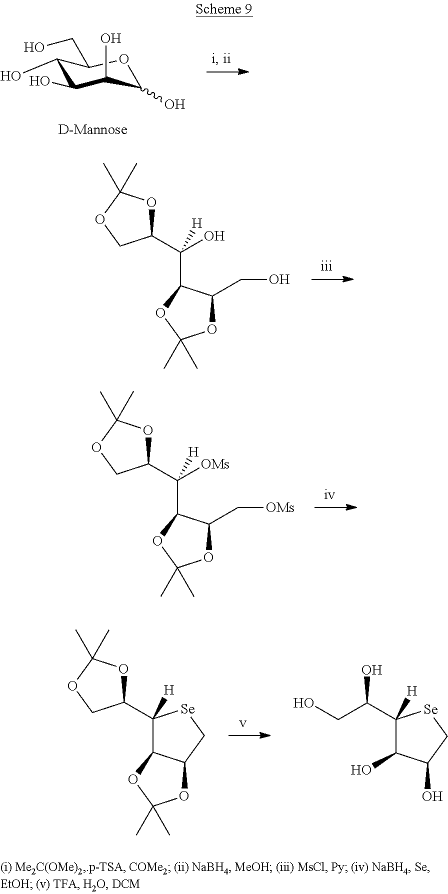

It will be appreciated from the above schemes, that various other seleno containing carbohydrates may be obtained by following the procedures using different starting carbohydrates.

During the reactions a number of the moieties may need to be protected. Suitable protecting groups are well known in industry and have been described in many references such as Protecting Groups in Organic Synthesis, Greene T W, Wiley-Interscience, New York, 1981.

In another aspect, the present invention provides pharmaceutical compositions for use as free-radical scavengers, more particularly as antioxidants, the composition comprising an effective amount of a seleno-compound of the present invention or a pharmaceutically acceptable salt thereof, and optionally a pharmaceutically acceptable carrier or diluent.

The term “composition” is intended to include the formulation of an active ingredient with encapsulating material as carrier, to give a capsule in which the active ingredient (with or without other carrier) is surrounded by carriers.

The pharmaceutical compositions or formulations include those suitable for oral, rectal, nasal, topical (including. buccal and sub-lingual), vaginal or parenteral (including intramuscular, sub-cutaneous and intravenous) administration or in a form suitable for administration by inhalation or insufflation.

The seleno-compounds of the invention, together with•a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to one hundred (100) milligrams, per tablet, are accordingly suitable representative unit dosage forms.

The seleno-compounds of the present invention can be administered in a wide variety of oral and parenteral dosage forms. It will be obvious to those skilled in the art that the following dosage forms may comprise, as the active component, either a compound of the invention or a pharmaceutically acceptable salt of a compound of the invention.

The compounds of the present invention may be administered to a subject as a pharmaceutically acceptable salt. It will be appreciated however that non-pharmaceutically acceptable salts also fall within the scope of the present invention since these may be useful as intermediates in the preparation of pharmaceutically acceptable salts. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valerie acids.

Base salts include, but are not limited to, those formed with pharmaceutically acceptable cations, such as sodium, potassium, lithium, calcium, magnesium, ammonium and alkylammonium.

For preparing pharmaceutical compositions from the compounds of the present invention, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

In powders, the carrier is a finely divided solid that is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it.

Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized moulds, allowed to cool, and thereby to solidify.

Formulations suitable for vaginal administration may be presented as pessaries, tampons, creams, gels, pastes, foams or prays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active•ingredient can be dissolved or suspended in a pharmaceutically acceptable carrier, such as sterile water, sterile organic solvent or a mixture of both.

The seleno-compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, eg. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention the compounds according to the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 5 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. •In such form, the preparation is subdivided into. unit doses containing appropriate quantities of the active component.• The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The invention also includes the compounds in the absence of carrier where the compounds are in unit dosage form.

The amount of the seleno-compound which is to be administered may be in the range from about 10 mg to 2000 mg per day, depending on the activity of the compound and the disease to be treated.

Liquids or powders for intranasal administration, tablets or capsules for oral administration and liquids for intravenous administration are the preferred compositions.

The compositions may further contain one or more other antioxidants or be administered along with another active agent such as for instance an antihypertensive agent.

As discussed above the present inventors have found that seleno-compounds act as effective oxidant scavengers in plasma. Accordingly, the components of the present invention may be used in therapies where antioxidants have proven to be effective such as treating conditions associated with oxidative stress.

Thus, in another aspect the invention provides a method for scavenging oxidants in plasma comprising administering to a subject an effective amount of a compound of formula (I).

Humans consume approximately 250 grams of oxygen per day and a typical human cell metabolises about 10¹² molecules of oxygen per day. An inevitable consequence of our dependence on oxygen is that small amounts of highly reactive radical and non-radical derivatives of diatomic oxygen (ROS), such as O₂.⁻, H₂O₂, .OH, RO₂., ROOH, HOCl, HOBr, HOSCN and ONOO⁻, are generated in vivo.

The main source of ROS within the arterial wall is a form of the enzyme NAD(P)H oxidase. This enzyme generated superoxide radicals by catalysing the reduction of O₂ (see scheme 10). Superoxide radicals can subsequently be converted to more potent ROS. For example, dismutation provides hydrogen peroxide and reaction with nitric oxide affords peroxynitrite (see scheme 10).

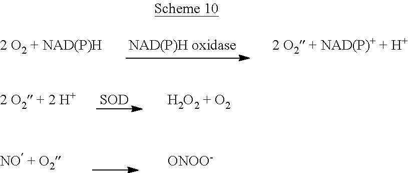

Living organisms utilise ROS as inter- and intracellular mediators of signal transduction. However, ROS can oxidise all major classes of biomolecules and are harmful at high concentrations. Living organisms are protected against ROS by a group of antioxidant compounds and enzymes. Notable antioxidant enzymes are the enzymes glutathione peroxidase (GPx) and thioredoxin reductase which both contain selenium.

Antioxidants prevent the formation of ROS or intercept ROS and exclude them from further activity. In healthy aerobic organisms, ROS production is counterbalanced by antioxidant defense networks and ROS levels are tightly regulated. However, sometimes the endogenous antioxidant defense network becomes overwhelmed by excess ROS. This imbalance between ROS and antioxidants in favour of ROS is referred to as oxidative stress and it has been implicated in the pathology of a vast array of diseases including, hyperlipidemia, diabetes mellitus, ischemic heart disease, atherosclerosis and chronic heart failure. There is a growing body of evidence which suggests that oxidative stress is also involved in the pathogenesis of hypertension. This is because one of the many effects of angiotensin II is to stimulate NAD(P)H oxidase and thereby increase the amount of NAD(P)H oxidase derived ROS present in the vasculature. The numerous mechanisms via which these ROS proceed to bring about hypertension are yet to be fully elucidated. It is thought that hydrogen peroxide may increase•the concentration of calcium cations in vascular cells and calcium cations are known to induce vasoconstriction. Alternatively, ROS may activate genes and transcription factors mediated oxidation of arachidonic acid to F₂-isoprostanes, which are prostaglandin-like compounds that are potent vasoconstrictors.

Accordingly, the seleno-compounds of the present invention may be useful in the treatment of conditions associates with oxidative stress. For instance, the compounds of the present invention may be useful in the treatment of neurodegenerative diseases and conditions such as Alzheimer's disease, Parkinson's disease, parkinsonian syndrome (multiple system atrophy and progressive supenuclear palsy), amyotrophic lateral sclerosis, dementia (including Lewy body dementia), •Friedrich's ataxia, Wilson's disease, Ataxia Telangiectasia, Motor neurone disease, Alexander disease, Alper's disease, Batten disease (also known as Spielmeyer-Vogt-Sjogreri-Batten disease), Canavan disease, Cockayne syndrome, Corticobasal degeneration, Creutzfeldt-Jakob disease, Huntington disease, Kennedy's disease, Krabbe disease, Machado-Joseph disease (Spinocerebellar ataxia type 3), Multiple sclerosis, Multiple System Atrophyl, Pelizaeus-Merzbacher Disease, Pick's disease, Primary lateral sclerosis, Refsum's disease, Sandhoff disease, Schilder's disease, Spinocerebellar ataxia (multiple types with varying characteristics), Spinal muscular atrophy, Steele-Richardson-Olszewski disease, and Tabes dorsalis.

Furthermore, mtDNA diseases such as cardiomyopathy, heart failure, heart block, arrhythmia, diabetes, pancreatitis, retinopathy, optic neuropathy, renal failure, Kearns Sayre Syndrome, Sudden Infant Death Syndrome, dementia and epilepsy, stroke may also be effectively treated using the compounds of the present invention.

Other conditions such as inflammation, ischaemic-reperfusion tissue injury in strokes, heart attacks, organ transplantation and surgery, edema, atherosclerosis, may also be beneficially treated with the compounds of the present invention.

For certain of the above mentioned conditions/diseases it is clear that the compounds may be used prophylactically as well as for the alleviation of acute symptoms. References herein to “treatment” or the like are to be understood to include such prophylactic treatment, as well as treatment of acute conditions.

From the above discussion it would be evident that one of the other main advantages of the seleno-compounds of the present invention will be their ability to provide cardio-protective qualities. Accordingly, the present seleno-compounds are seen to be beneficial in the context of increasing the bodies natural ability to prevent (or enhance: the prevention of) tissue damage in the cardiovascular system.

The invention will now be described in the following Examples. The Examples are not to be construed as limiting the invention in any way.

### EXAMPLES

### 1.1 Synthetic Examples

### General Experimental Techniques

¹H NMR spectra were recorded on Varian (nova 400 (400 MHz) or Varian (nova 500 (500 MHz) instruments at room temperature, using CDCl₃ (or other indicated solvents) as internal reference deuterium lock, CDCl₃ at δ 7.26 ppm, CH₃OD at δ 3.31 ppm. The chemical shift data for each signal are given as δ in units of parts per million (ppm). The multiplicity of each signal is indicated by: s (singlet), br s (broad singlet), d (doublet), t (triplet), q (quartet); dd (doublet of doublets), dt (doublet of triplets) and m (multiplet). The number of protons (n) for a given resonance is indicated by n H. Coupling constants (J) are quoted in Hz and are recorded to the nearest 0.1 Hz.

¹³C NMR spectra were recorded on Varian (nova 400 (400 MHz) or Varian Inova 500 (500 MHz) instruments using the central resonance of the triplet of CDCl₃ at δ 77.23 ppm as an internal reference. The chemical shift data for each signal are given as δ in units of parts per million (ppm).

⁷⁷Se NMR spectra were recorded on a Varian (nova 500 (500 MHz) instrument with proton decoupling. The chemical shift data for each signal are given as δ in units of ppm relative to (SePh)₂.

Infrared spectra were recorded on a Perkin Elmer Spectrum One FT-IR spectrometer in the region 4000-650 cm⁻¹. The samples were analysed as thin films from dichloromethane or as solutions in the indicated solvents.

Mass spectra were recorded at the Bio21 Institute, The University of Melbourne. Low resolution spectra were recorded on a Waters Micromass Quattro II instrument (El and Cl). All high resolution mass spectrometry experiments were conducted using a commercially available hybrid linear ion trap and Fourier transform ion cyclotron resonance (FT-ICR) mass spectrometer (Finigan LTQ-FT San Jose, Calif.), which is equipped with ESI. The ions of interest were mass selected in the LTQ using standard procedures and were then analyzed in the FT-ICR MS to generate the high resolution tandem mass spectrum.

Optical specific rotations were measured using a Jasco DIP-1000 digital polarimeter, in a cell of 1 dm path length. The concentration (c) is expressed in g/100 cm³ (equivalent to g/0.1 dm³). Specific rotations are denoted [α]_{D}^{T} and given in implied units of °dm² g⁻¹ (T=temperature in ° C.).

Analytical thin layer chromatography (TLC) was carried out on pre-coated 0.25 mm thick Merck 60 F₂₅₄ silica gel plates. Visualisation was by absorption of UV light, or thermal development after dipping in an ethanolic solution of phosphomolybic acid (PMA) or sulfuric acid (H₂SO₄). Flash chromatography was carried out, on silica gel [Merck Kieselgel 60 (230-400 mesh)] under a pressure of nitrogen.

Hydrogenation was carried out in a Büchi GlasUster “miniclave drive” stainless steel vessel, 100 ml, with a maximum operation pressure of 60 bar. Teflon inserts were used and reactions were stirred using magnetic stirrer bars.

Dry DMF was distilled from sodium hydride. Anhydrous THF, diethyl ether, and dichloromethane were dried by passage through a packed column of activated neutral alumina under a nitrogen atmosphere, and toluene being passed through a column with additional R3-11 copper-based catalyst (BASF Australia). Petroleum ether refers to the fraction of boiling point range 40-60° C. Procedures using moisture or air sensitive reagents were undertaken in a nitrogen-filled dual manifold employing standard Schlenk line techniques.

Melting points were determined with an Electrothermal Engineering IA9100 or a Büchi 510 melting point apparatus and are uncorrected.

### Synthesis of Selenium Containing Carbohydrates

### Synthesis of 1,5-anhydro-5-seleno-L-gulitol

### 2,3,4,6-Di-isopropylidene-1,5,-di-O-hydroxy-D-mannitol (1)

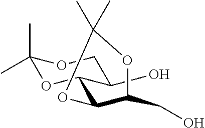

To a suspension of D-mannose (10 g, 55.5 mmol) and p-toluenesulfonic acid monohydrate (1.06 g, 5.55 mmol) over 4 Å molecular sieves in dry DMF (100 mL) at 0° C. was added 2-methoxypropene (10.6 mL, 8.0 g, 222 mmol) dropwise over 30 minutes. The suspension was maintained at 0° C. for 8 hours and allowed to warm to room temperature. The resulting pale yellow solution was quenched by the addition of NaCO₃ (2 g). Filtration and removal of the solvent in vacuo gave a yellow oil. The residue was partitioned between ethyl acetate (200 mL) and water (200 mL) and the organic layer was separated. The aqueous phase was extracted with ethyl acetate (3×100 mL) and the combined organic extracts washed with brine (2×80 mL) and dried over MgSo₄. Evaporation afforded the crude di-isopropylidene as the major of three products, two of which were virtually inseparable by column chromatography R_{f}0.18 (hexane:ethyl acetate)(3:1). The crude mixture was then dissolved in anhydrous methanol (100 mL) under nitrogen at 0° C. before the portionwise addition of sodiumborohydride (2.9 g, 77 mmol). Vigorous effervescence occurred and the solution was stirred at 0° C. for 30 min and then at room temperature for 4 hours. Two new products were observed by TLC, the major of which being the desired diol 1 (R_{f}0.36), the minor product (R_{f}0.52) (ethyl acetate:hexanes) (2:1) was now able to be separated by column chromatography The solvent was removed in vacuo and the residue was partitioned between ethyl acetate (150 mL) and water (150 mL) and the organic layer was separated. The aqueous phase was extracted with ethyl acetate (5×50 mL) and the combined organic extracts washed with brine (2×50 mL) and dried over MgSO₄. Evaporation and chromatography (25%-67% ethyl acetate in petroleum ether) afforded the diol (1) as a colourless oil (9.36 g, 36 mmol, 67% over 2 steps). R_{f}0.36 (Hex:EtOAc 1:2); [α]_{D}²²=−12.8° (c 1.0 in DCM); ¹H NMR (500 MHz, CDCl₃) δ 4.47 (dd, J=2.3, 6.7 Hz, 1H), 4.31 (dt, J=4.8, 6.7 Hz, 1H), 3.97-3.89 (m, 2H), 3.81 (m, 2H), 3.70 (dd, J=2.3, 8.8 Hz, 1H), 3.64 (td, J=2.6, 10.3•Hz, 1H), 1.53 (s, 3H), 1.49 (s, 3H), 1.41 (s, 3H), 1.38 (s, 3H); ¹³C NMR (125 MHz, CDCl₃) δ 109.10, 99.43, 77.88, 74.90, 72.58, 64.94, 63.98, 61.66, 28.29, 26.90, 25.80, 19.65; IR(neat)/cm⁻¹: 3433, 2986, 1217, 1066; MS (ESI⁺) m/z (rel intensity) 263.09 [100, (M+Na)⁺]; HRMS (ESI⁺) m/z 263.1489 (263.1489 calcd for C₁₂H₂₂O₆Na). These data agree with the published literature values (H. Liu and B. M. Pinto, Can. J Chern., 2006, 84, 4, 497-505).

### 2,3,4,6-Di-O-isopropylidene-1,5-di-O-methanesulfonyl-D-mannitol (2)

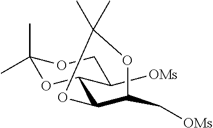

To a stirred solution of the diol (1)(5 g, 19 mmol), 4-dimethylaminopyridine (DMAP, 250 mg, 2 mmol) and anhydrous pyridine (10 mL) in dry CH₂Cl₂ (150 mL) under nitrogen at 0° C. was added dropwise methanesulfonyl chloride (4.5 mL, 59 mmol). The solution was stirred at 0° C. for 30 min and then warmed to room temperature for 6 hours. The reaction was quenched by the addition of saturated NaHCO₃ (50 mL) before being extracted with CH₂Cl₂ (3×50 mL). The combined organic extracts were then washed with brine (2×100 mL) and dried over MgSO₄. Evaporation and chromatography (25% ethyl acetate in hexane) afforded the dimesylate (2) as a white crystalline solid (6.76 g, 16 mmol, 85%). R_{f}0.15 (Hex:EtOAc 3:1); [α]_{D}²²=+19.9° (c 1.0 in DCM) (Lit+2.4° c. 0.5 in DCM); ¹H NMR (500 MHz, CDCl₃) δ 4.82 (ddd, J=5.1, 7.3, 8.8 Hz, 1H), 4.55 (ddd, J=4.1, 6.3, 7:5 Hz, 1H), 4.50 (dd, J=7.5, 10.3 Hz, 1H) 4.40 (dd, J=1.1, 6.3 Hz, 1H), 4.38 (dd, J=4.1, 10.3 Hz, 1H), 4.13 (dd, J=5.1, 12.0 Hz, 1H), 3.88 (dd, J=7.3, 12.1 Hz, 1H), 3.81 (dd, J=1.1, 8.8 Hz, 1H), 3.08 (s, 3H), 3.07 (s, 3H), 1.52 (s, 3H), 1.50 (s, 3H), 1.41 (s, 3H), 1.37 (s, 3H); ¹³C NMR (125 MHz, CDCl₃) δ 110.46, 100.01, 74.87, 73.86, 72.25, 69.55, 68.19, 62.62, 38.12, 38.04, 27.36, 26.80, 25.84, 20.32; IR (neat)/cm⁻¹: 2970, 1738, 1365, 1217; MS (ESI⁺) m/z (rel intensity) 441.18 [100, (M+Na)⁺]; HRMS (ESI⁺) m/z 441.0860 (441.0859 calcd for C₁₄H₂₆O₁₀S₂Na). These data agree with the published literature values (H. Liu and B. M. Pinto, Can. J Chern., 2006, 84, 4, 497-505).

### 2,3,4,6-Di-O-isopropylidene-1,5-anhydro-5 seleno-L-gulitol (3)

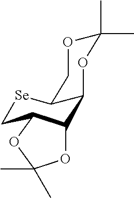

To a stirred suspension of the selenium powder (0.85 g, 10.8 mmol) in degassed ethanol (40 mL) under argon at 0° C. was added a saturated solution of sodiumborohydride (˜1 g) in 5 degassed ethanol (10 mL). The suspension was stirred at 0° C. for 10 min and at room temperature for 1 h during which time the black selenium colour disappeared. The clear solution was then cooled to 0° C. for the addition of the dimesylate (2) (3 g, 7.2 mmol) in THF (5 mL). The reaction mixture was heated and stirred at 70° C. for 12 hours. The solvent was removed in vacuo before the residue was partitioned between ethyl acetate (50 mL) and water (50 mL) and the organic layer was separated. The aqueous phase was extracted with ethyl acetate (3×30 mL) and the combined organic extracts were washed with brine (2×30 mL) and dried over MgSO₄. Evaporation and chromatography (25% ethyl acetate in petroleum ether) afforded the seleno-gulitol (3) as a white crystalline solid (1.34 g, 4.4 mmol, 61%). R_{f}0.49 (Hex:EtOAc 3:1); [α]_{D}²²=−33.1° (c 0.5 in DCM) (Lit −32° c. 0.5 in DCM); ¹H NMR (500 MHz, CDCl₃) 4.43 (ddd, J=2.4, 5.4, 5.8 Hz, 1H), 4.37 (t, J=3.2 Hz, 1H), 4.22 (dd, J=2.7, 12.6 Hz, 1H) 4.13 (J=3.5, 5.9 Hz, 1H), 3.78 (dd, J=2.0, 12.7 Hz, 1H), 3.21 (dd, J=3.3, 12.6 Hz, 1H), 3.18 (dd, J=2.0, 2.7, 6.9 Hz, 1H), 2.68 (dd, J=5.9, 12.6 Hz, J_{H,Se}=12.4 Hz, 1H), 1.53 (s, 3H), 1.47 (s, 3H), 1.45 (s, 3H), 1.36 (s, 3H); ¹³C NMR (125 MHz, CDCl₃) δ 108.48, 99.48, 75.44, 70.29, 67.67, 65.01, 29.49, 28.22, 27.00, 25.16, 20.99, 19.37; ⁷⁷Se NMR (95 MHz, CDCl₃) δ 63; IR (neat)/cm⁻¹:2970, 1739, 1366, 1217; MS (ESI⁺) m/z (rel intensity) 360.55 [100, (M+53⁺]; HRMS (ESI⁺) m/z 331.0420 (331.0419 calcd for C₁₂H₂₀O₄SeNa). Anal. Calcd. for C₁₂H₂₀O₄Se: C, 46.92; H, 6.56; O, 20.84; Se, 25.68. Found: C, 47.02; H, 6.49; O, 20.90. These data agree with the published literature values (H. Liu and B. M. Pinto, Can. J Chern., 2006, 84, 4, 497-505).

### 1,5-Anhydro-5-seleno-L-gulitol (4)

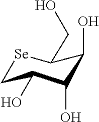

To a stirred solution of the protected seleno sugar (0.5 g, 1.6 mmol) in dry methanol (10 mL) under nitrogen at 0° C. was added acetyl chloride (0.5 mL). The solution was stirred at 0° C. for 10 min and at room temperature for 3 h. The solvent was removed in vacuo and the residue was purified by column chromatography (30% methanol in dichloromethane) afforded the deprotected seleno sugar (4) as a white crystalline solid (0.21 g, 0.91 mmol, 57%). R_{f}0.50 (MeOH:EtOAc 1:5); [α]_{D}²²=−17.7° (c 0.1 in MeOH); ¹H NMR (500 MHz, CD₃OD) δ 4.15 (ddd, J=2.5, 4.0, 11.4 Hz, 1H), 4.12 (dd, J=2.5, 5.4 Hz, 1H), 3.80 (dd, J=1.6, 5.3 Hz, 1H), 3.76 (dd, J=7.2, 11.0 Hz, 1H), 3.65 (dd, J=6.8, 11.0 Hz, 1H), 3.59 (td, J=2.0, 7.0 Hz, 1H), 3.04 (t, J=11.3 Hz, 1H), 2.28 (dd, J=3.9, 11.5 Hz, J_{H,Se}=12.4 Hz, 1H); ¹³C NMR (125 MHz, CDCl₃) δ 73.69, 72.32, 68.83, 63.42, 39.04, 19.21; ⁷⁷Se NMR (95 MHz, MeOD) δ 81.1; IR (neat)/cm⁻¹: 3321, 2126, 1638; MS (ESI⁺) m/z (rel intensity) 360.45 [100, (M+133.33)⁺]; HRMS (ESI⁺) m/z 250.9793 (250.9799 calcd for C₆H₁₂O₄SeNa); Anal. Calcd. for C₆H₁₂O₄Se: C, 31.74; H, 5.33; O, 28.19; Se, 34; 74. Found: C, 32.01; H, 5.15.

### Synthesis of 1,5-anhydro-5-seleno-D-mannitol

### 2,3,4,6-Di-isopropylidene-1-tert-butyl-dimethylsilyl-5-O-hydroxy-D-mannitoi (5)

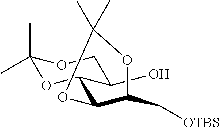

To a solution of the dial (1) (5 g, 19.1 mmol) in dry CH₂Cl₂ (100 mL) under nitrogen at 0° C. was added imidazole (3.2 g; 47.7 mmol) followed by TBDMSCI (3.16 g, 21.0 mmol). The solution was stirred at 0° C. for 10 minutes and was then allowed to warm to room temperature and stirred for 2 hours, during which time a solid white precipitate formed. The reaction mixture was then diluted with CH₂Cl₂ (100 mL) and poured into water (100 mL). The organic fraction was washed with saturated NaHCO₃ (2×40 mL), dried over MgSO₄ and concentrated to afford a viscous clear yellow oil. Flash chromatography (25% ethyl acetate in petroleum ether) afforded the silyl ether (5) as a colourless oil (6.82 g, 18.1 mmol, 95%). R_{f}0.48 (Hex:EtOAc 3:1); [α]_{D}²²=−58.7° (c 1.0 in DCM); ¹H NMR (500 MH_{z}, CDCl₃) δ 4.37 (dd, J=3.1, 6.2 Hz, 1H) 4.32 (dd, J=5.8, 13.1, Hz, 1H), 3.92-3.78 (m, 5H), 3.64-3.57 (m, 1H), 2.77 (bs, 1H), 1.48 (s, 6H), 1.41 (s, 3H), 1.37 (s, 3H), 0.91 (s, 9H). 0.10 (s, 6H); ¹³C NMR (125 MHz, CDCl₃) δ 109.02, 98.81, 77.51, 76.06, 72.42, 65.12, 64.43, 62.17, 28.61, 27.17, 26.05, 19.60, 18.51, −5.16, −5.22; IR (neat)/cm⁻¹: 3530, 2985, 2930, 1379, 1251, 1075; MS (ESI⁺) m/z (rel intensity) 449.18 [100, (M+72⁺]; HRMS (ESI⁺) m/z 377.2353 (377.2354 calcd for C₁₈H₃₆O₆Si); Anal. Calcd. for C₁₈H₃₆O₆Si: C, 57.41; H, 9.64; O, 25.49; Si, 7.46. Found: C, 57.27; H, 9.48.

### 2,3,4,6-Di-isopropylidene-1-tert-butyl-dimethylsilyl-5-O-hydroxy-L-gulitol (6)

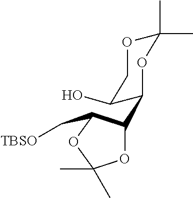

To a solution of the DMSO (150 μL, 2.2 mmol) in dry CH₂Cl₂ (15 mL) under nitrogen at −78° C. was added oxalyl chloride (140 μL, 1.6 mmol) drop wise. The solution was stirred at −78° C. for 30 minutes before the drop wise addition of the alcohol (5) (200 mg, 0.53 mmol) in CH₂Cl₂ (5 mL). The mixture was stirred for a further hour at −78° C. before the addition of Et₃N (600 μL, 4.3 mmol). After stirring for a further 30 minutes at −78° C. the starting material had disappeared by TLC and the solution was warmed to room temperature. Following dilution with CH₂Cl₂ (50 mL) and addition of saturated NaHC0₃ (50 mL). The organic layer was separated and the aqueous layer was extracted with further dichloromethane (2×50 mL). The combined organic fractions were washed gain with saturated H₂O (100 mL), then brine (100 mL) and dried over MgSO₄. Evaporation of the solvent yielded a colourless oil. The oil was dried under vacuum for 2 hours before being dissolved in dry methanol (20 mL) and cooled to −78° C. Anhydrous cerium (III) chloride (200 mg, 0.8 mmol) was added before the portionwise addition of NaBH₄ (20 mg, 0.56 mmol). The solution was stirred for 10 minutes until TLC showed full consumption of starting material. The solution was then warmed to room temperature and concentrated in vacuo. The remaining residue was diluted with water (75 mL), and extracted with ethyl acetate (3×75 mL). The combined organic fractions were washed with saturated NaCl (2×50 mL), dried over MgSO₄ and concentrated to afford a viscous clear oil. Flash chromatography (25% ethyl acetate in petroleum ether) afforded the alcohol (6) as a colourless oil (170 mg, 45 mmol, 85%). R_{f}0.38 (Hex:EtOAc 3:1); [α]_{D}²²−10.7° (c 1.0 in DCM); ¹H NMR (500 MHz, CDCl₃) δ 4.40 (t, J=5.7 Hz, 1H), 4.22 (ddd, J=8.4, 5.7, 4.3 Hz, 1H), 4.10 (dd, J=5.8, 1.3 Hz, 1H), 4.00 (dd, J=12.2, 1.5 Hz, 1H), 3.82-3.76 (m, 1H), 3.76-3.72 (m, 1H), 3.70 (dd, J=10.5, 4.3 Hz, 1H), 1.49 (s, 6H), 1.47 (s, 3H), 1.37 (s, 3H), 0.89 (s, 9H), 0.07 (d, J=0.7 Hz, 6H); ¹³C NMR (125 MHz, CDCl₃) δ 109.11, 99.02, 78.22, 76.88, 69.41, 65.59, 65; 17, 61.97, 29.51, 27.37, 26.05, 25.84, 25.70, 18.54, −5.16, −5.22; IR (neat)/cm⁻¹: 3525, 2990, 2931, 1380, 1252, 1074; MS (ESI⁺) m/z (rel intensity) 399.17 [100, (M+Nat]; HRMS (ESI⁺) m/z 399.2173 (399.2173 calcd for C₁₈H₃₆O₆SiNa). Anal. Calcd. for C₁₈H₃₆O₆Si: C, 57.41; H, 9.64; O, 25.49; Si, 7.46. Found: C, 57.35; H, 9.49.

### 2,3,4,6-Di-isopropylidene-1,5-di-O-hydroxy-L-gulitol (7)

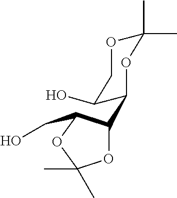

To a stirred solution of (6) (3 g, 7.97 mmol) in dry THF (50 mL) under nitrogen at room temperature was added TBAF (7.4 mL of a 1.0M solution in THF, 7.4 mmol) dropwise. After 1 hour the mixture was diluted with ethyl acetate (200 mL) and washed with water (2×100 mL) followed by brine (100 mL). Drying over MgSO₄ and concentration in vacuo afforded compound (7) as a clear colourless oil (1.88 g, 7.17 mmol, 90%). R_{f}0.18 (EtOAc); [α]_{D}²²=−3.6° (c 1.0 in DCM); ¹H NMR (500 MHz, CDCl₃) δ 4.40 (dd, J=6.3, 4.5 Hz, 1H), 4.33-4.27 (m, 1H), 4.03 (ddd, J=5.8, 3.0, 0.9 Hz, 2H), 3.84 (dd, J=12.4, 2.1 Hz, 1H), 3.79-3.73 (m, 2H), 3.61 (ddd, J=6.7, 3.6, 1.6 Hz, 1H), 1.55 (s, 3H), 1.50 (s, 6H), 1.40 (s, 3H); ¹³C NMR (125 MHz, CDCl₃) δ 109.44, 99.28, 78.02, 77.77, 69.31, 65.58, 65.53, 61.37, 29.38, 27.14, 25.66, 18.46; IR (neat)/cm⁻¹: 3449, 2989, 2926, 1381, 1221, 104l; MS (ESI⁺) m/z (rel intensity) 285.18 [100, (M+Na)⁺]; HRMS (ESI⁺) m/z 285.1309 (285.1309 calcd for C₁₂H₂₂O₆Na); Anal. Calcd. for C₁₂H₂₂O₆: C, 54.95; H, 8.45. Found: C, 54.97; H, 8.43.

### 2,3,4,6-Di-isopropylidene-1,5-di-O-methanesulfonyl-L-gulitol (8)

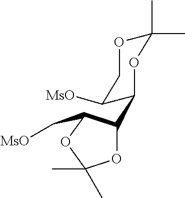

To a stirred solution of the dial (7) (5 g, 19 mmol), 4-dimethylaminopyridine (DMAP, 250 mg, 2 mmol) and anhydrous pyridine (10 mL) in dry CH₂Cl₂ (150 mL) under nitrogen at 0° C. was added dropwise methanesulfonyl chloride (4.5 mL, 59 mmol). The solution was stirred at 0° C. for 30 min and then warmed to room temperature for 6 hours. The reaction was quenched by the addition of saturated NaHCO₃ (50 mL) before being extracted with CH₂Cl₂ (3×50 mL). The combined organic extracts were then washed with brine (2×100 mL) and dried over MgSO₄. Evaporation and chromatography (25% ethyl acetate in hexane) afforded the dimesylate (8) as a white crystalline solid (6.76 g, 16 mmol, 85%). R_{f}0.36 (Hex:EtOAc 1:1); [α]_{D}²²=+15.2° (c 1.0 in DCM); ¹H NMR (500 MHz, CDCl₃) δ 4.75 (q, J=2.1 Hz, 1H), 4.56-4.50 (m, 1H), 4.43 (dd, J=7.3, 5.3 Hz, 1H), 4.37 (dd, J=10.7, 6.7 Hz, 1H), 4.28 (dd, J=7.3, 1.7 Hz, 1H), 4.22 (dd, J=10.7, 4.8 Hz, 1H), 4.18 (dd, J=13.6, 2.0 Hz, 1H), 4.09 (dd, J=13.6, 2.3 Hz, 1H), 3.18 (s, 3H), 3.10 (s, 3H), 1.55 (s, 3H), 1.53 (s, 3H), 1.51 (s, 3H), 1.42 (s, 3H); ¹³C NMR (126 MHz, CDCl₃) δ 109.60, 99.69, 76.28, 74.67, 73.49, 71.24, 69.36, 68.12, 67.94, 67.17, 62.68, 62.42, 39.12, 37.93, 37.73, 28.75, 27.86, 26.58, 25.62, 25.49, 20.13, 18.85; IR (neat)/cm⁻¹: 2992, 2940, 1351, 1173; MS (ESI⁺) m/z (rel intensity) 436.27 [100, (M+18+]; HRMS (ESI⁺) m/z 441.0860 (441.0860 calcd for C₁₄H₂₆O₁₀S₂Na); Anal. Calcd. for C₁₄H₂₆O₁₀S₂: C, 40.18; H, 6.26. Found: C, 40.09; H, 6.28.

### 2,3,4,6-Di-O-isopropylidene-1,5-anhydro-5-seleno-D-mannitol (9)

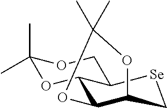

To a stirred suspension of the selenium powder (1 g, 12.7 mmol) in degassed ethanol (40 mL) under argon at 0° C. was added a saturated solution of sodiumborohydride (˜1 g) in degassed ethanol (10 mL). The suspension was stirred at 0° C. for 10 min and at room temperature for 1 h during which time the black selenium colour disappeared. The clear solution was then cooled to 0° C. for the addition of the dimesylate (8) (3 g, 7.2 mmol) in THF (5 mL). The reaction mixture was heated and stirred at 70° C. for 12 hours. The solvent was removed in vacuo before the residue was partitioned between ethyl acetate (50 mL) and water (50 mL) and the organic layer was separated. The aqueous phase was extracted with ethyl acetate (3×30 mL) and the combined organic extracts were washed with brine (2×30 mL) and dried over MgSO₄. Evaporation and chromatography (25% ethyl acetate in petroleum ether) afforded the seleno-gulitol (9) as a white crystalline solid (1.34 g, 4.4 mmol, 61%). R_{f}0.51 (Hex:EtOAc 3:1) [α]_{D}²²=+21.3° (c 1.0 in DCM); ¹H NMR (500 MHz, CDCl₃) δ 4.53-4.44 (m, 1H), 4.11-3.97 (m, 3H), 3.85 (dd, J=11.6, 5.1 Hz, 1H), 3.13 (td, J=11.2, 5.2 Hz, 1H), 2.83 (t, J=11.3 Hz, 1H), 2.63 (dd, J=11.5, 4.5 Hz, J_{H,Se}=12.4 Hz, 1H), 1.57. (s, 3H), 1.51 (s, 3H), 1.46 (s, 3H), 1.38 (s, 3H); ¹³C NMR (125 MHz, CDCl₃) δ 109.07, 99.45, 77.49, 75.72, 73.49, 64.55, 29.61, 27.67, 27.58, 25.03, 19.14, 18.01; ⁷⁷Se NMR (95 MHz; CDCl₃) δ 103.02; IR (neat)/cm⁻¹: 2990, 2938, 1373, 1198, 1057; MS (ESI⁺) m/z (rel intensity) 251.18 [100, (M-56)⁺]; HRMS (ESI⁺) m/z 414.9574 (414.9572 calcd for C₁₂H₂₀O₄Ag); Anal. Calcd. for C₁₂H₂₀O₄Se: C, 45.92; H, 6.56. Found: C, 47.01; H, 6.52.

### 1,5-Anhydro-5-seleno-D-mannitol (10)

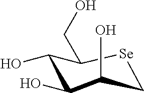

To a stirred solution of the protected seleno-sugar (0.5 g, 1.6 mmol) in dry methanol (10 mL) under nitrogen at 0° C. was added acetyl chloride (0.5 mL). The solution was stirred at 0° C. for 10 min and at room temperature for 3 h. The solvent was removed in vacuo and the residue was purified by column chromatography (30% methanol in dichloromethane) afforded the deprotected seleno-sugar (10) as a white amorphous solid (0.21 g, 0.91 mmol, 57%). R_{f}0.29 (EtOAc:MeOH 5:1); [α]_{D}²²=−41.4° c. 0.1 in MeOH; ¹H NMR (500 MHz, CD30D) δ 4.30 (td, J=5.5, 3.2 Hz, 1H), 4.24 (dd, J=5.3, 3.2 Hz, 1H), 3.70 (ddd, J=9.2, 6.1, 3.3 Hz, 1H), 3.63 (dd, J=11.4, 3.3 Hz, 1H), 3.48 (ddd, J=11.4, 6.1, 0.6 Hz, 1H), 3.42 (dd, J=8.6, 5.4 Hz, 1H), 2.98 (dd, J=9.9, 5.1 Hz, 1H), 2.79 (dd, J=9.9; 6.0 Hz, J_{H,Se}=12.4 Hz, 1H); ¹³C NMR (126 MHz, CD₃OD) δ 78.50, 76.04, 75.10, 65.27, 44.53, 23.84; ⁷⁷Se NMR (95 MHz, CD₃OD) δ 97.9; IR (neat)/cm⁻¹: 3346, 2885, 1415, 1051; MS (ESI⁺) m/z (rel intensity) 243.17 [100, (M+I6)⁺]; HRMS (ESI⁺) m/z 250.9793 (250.9793 calcd for C₆H₁₂O₄SeNa); Anal. Calcd. for C₆H₁₂O₄Se: C, 31.74; H, 5.33. Found: C, 31.63; H, 5.33.

### Synthesis of 1,5-anhvdro-5-seleno-L-iditol

Bromo-2,3,4,6-tetra-O-acetyl-α-D-glucopyranoside (11)

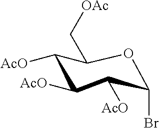

To a suspension of D-Glucose (10 g, 83 mmol) in acetic anhydride (33 mL) under nitrogen at room temperature was added hydrobromic acid (8 ml, 33% HBr in acetic acid) dropwise. The suspension was stirred for 1 hour during which time the glucose dissolved into the solution. After this time a further amount of hydrobromic acid was added (42 mL, 33% HBr in acetic acid) dropwise and the reaction was stirred at room temperature overnight. Sodium acetate (10 g) was then added and the solution was stirred for a further 30 minutes before the addition of CH₂Cl₂ (200 mL). The organic layer was washed with saturated NaHC03 (5×100 mL), brine (50 mL), dried over MgSO₄, and concentrated in vacuo to afforded desired product (11) (21.7 g, 79 mmol, 95%), which was used without further purification.

### Phenyl-2,3,4,6-Tetra-O-acetyl-1-seleno-β-D-glucopyranoside (12)

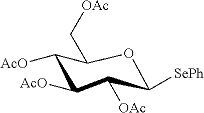

To a stirred suspension of the diphenyl diselenide (3.8 g, 12 mmol) in degassed ethanol (130 mL) under argon at 0° C. was added a saturated solution of sodiumborohydride (˜1 g) in degassed ethanol (20 mL). The suspension was stirred at 0° C. for 10 min and at room temperature for 1 h during which time the yellow colour disappeared. The bromide 11 (10 g, 24 mmol) in ethanol (100 mL) was then added dropwise before the reaction was stirred at room temperature for 2.5 hour. The reaction was quenched by the addition of glacial acetic acid (10 mL). One third of the solvent was removed in vacuo before the remaining solution was chilled in the freezer for 3 days during which time the product crystallized and was filtered off and washed with ice cold ethanol, giving the desired seleno glycoside (12) as a white crystalline solid (10.56 g, 21.6 mmol, 90%). R_{f}(Hex:EtOAc 3:1) 0.32; [α]_{D}²²=−29.1° (c 0.1 in DCM) (Lit [α]_{D}²¹−25° C. 0.1 in DCM); ¹H (500 MHz, CDCl₃) δ 7.61 (dd, J=1.3, 8.2 Hz, 2H), 7.31 (ddd, J=7.2, 13.2, 14.1 Hz, 3H), 5.19 (t, J=9.3 Hz, 1H), 5.06 (t, J=9.3 Hz, 1H), 5.00 (dd, J=5.3, 6.2 Hz, 1H), 4.89 (d, J=10.2, J_{H,Se}=15.8 Hz, 1H), 4.24-4.14 (m, 2H), 3.69 (ddd, J=2.8, 4.7, 10.1 Hz, 1H), 2.07 (s, 3H), 2.07 (s, 3H), 2.01 (s, 3H), 1.98 (s, 3H); ¹³C NMR (125 MHz, CDCl₃) δ 170.76, 170.37, 169.58, 169.49, 135.48, 129.22, 128.79, 127.13, 81.13, 77.08, 74.03, 70.99, 68.37, 62.31, 21.00, 20.94, 20.81, 20.78 ⁷Se NMR (95 MHz, CDCl₃) δ 422; IR(neat)/cm⁻¹ 2954, 1746, 1217, 1040; MS (ESI⁺) m/z (rel intensity) 595.18 [100, (M+Ag)⁺]; HRMS (ESI⁺) m/z 594.9635 (594.9628 calcd for C₂₀H₂₄O₉SeAg). These data agree with the published literature values (R. V. Stick, D. M. G. Tilbrook and S. J. Williams; Aust. J. Ch.em., 1997, 50, 3, 233-235).

### Phenyl-2,3,4,6-tetra-O-hydroxy-1-seleno-β-D-glucopyranoside (13)

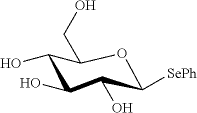

To a solution of the protected glycoside (12) (5 g, 10.2 mmol) in anhydrous methanol (200 mL) at 0° C. under nitrogen was added portionwise sodium metal (1 g, 43 mmol). The solution was then warmed to room temperature and stirred for 1 hour, until complete consumption of starting material had occurred by TLC analysis. The reaction was quenched with the addition of acidified amberlite ion exchange resin (IR 120) until the pH of the solution was acidic. Filtration through a thin celite plug followed by removal of the solvent yielded a light brown oil, which was then dried by co-evaporation with toluene to give the desired deprotected glycoside (13) (2.87 g, 9 mmol, 88%), without the need for further purification. R_{f}(EtOAc:MeOH 5:1) 0.62; [α]_{D}²²=−45.6° (c 10 in MeOH); ¹H NMR (500 MHz, CD₃OD) δ 7.68 (dd, J=5.5, 2.3 Hz, 2H), 7.39-7.18 (m, 3H), 4.82 (dd, J=9.8, 1.1 Hz, 1H), 3.85 (d, J=12.1 Hz, 1H), 3.65 (dd, J=12.2, 4.1 Hz, 1H), 3.35 (dd, J=12.9, 4.4 Hz, 1H), 3.29-3.19 (m, 3H); ¹³C NMR (125 MHz, CD₃OD) δ 133.95, 128.52, 127.28, 84.65, 81.80, 78.18, 73.07, 69.94, 61.48, 48.10, 47.93, 47.76, 47.59, 47.42, 47.24, 47.07; IR(neat)/cm⁻¹ 3330, 2881, 1437, 1019; Anal. Calcd. for C₁₂H₁₆O₅Se+H₂0: C, 42.74; H, 5.38. Found: C, 43.15; H, 5.48. These data agree with the published literature values (R. V. Stick, D. M. G. Tilbrook and S. J. Williams, Aust. J Chern., 1997, 50, 3, 233-235).

### Phenyl-2,3,4,6-tetra-O-p-methoxybenzyl-1-seleno-β-D-glucopyranoside (14)

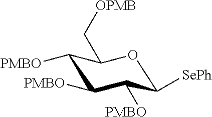

To a stirred suspension of sodium hydride (2 g, 83 mmol) in anhydrous DMF (70 mL) under nitrogen at 0° C. was added the deprotected glycoside (13) (2.8 g, 8.77 mmol) in dry DMF (30 mL) dropwise. The suspension was stirred at 0° C. for 30 min and was then allowed to slowly warm to room temperature and was stirred for a further 30 min until gas evolution had subsided. 4-Methoxybenzyl chloride (10.3 g, 9.4 mL, 79 mmol) was added under nitrogen at 0° C. and the reaction mixture was stirred at room temperature overnight. The clear yellow reaction mixture was quenched•by the addition of ethanol (10 mL), followed by water (5 mL) and the solvent was removed in vacuo. The residue was partitioned between ethyl acetate (100 mL) and water (100 mL) and the organic layer separated. The aqueous phase was extracted with ethyl acetate (3×50 mL). The combined organic extracts were washed with brine (100 mL). and dried over MgSO₄. Evaporation and flash column chromatography eluting with 25-100% ethyl acetate in petroleum ether afforded the protected glycoside (14) as a white solid (5.75 g, 7.19 mmol, 82%). R_{f}(Hex:EtOAc 3:1) 0.23; [α]_{D}²²=±2.8° (c 10 in DCM); ¹H NMR (500 MHz, CDCl₃) δ 7.68 (dd, J=8.2, 1.3 Hz, 2H), 7.32 (d, J=8.7 Hz, 2H), 7.25 (dd, J=12.9, 5.4 Hz, 7H), 7.20 (d, J=7.5 Hz, 2H), 7.09 (d, J=8.7 Hz, 2H), 6.85 (ddd, J=27.1, 14.0, 4.8 Hz, 9H), 4.80 (ddd, J=18.8, 10.2, 5.3 Hz, 4H), 4.72 (d, J=10.4 Hz, 1H), 4.66. (d, J. 9.9 Hz, 1H), 4.49 (dt, J=14.8, 11.6 Hz, 3H), 3.82-3.78 (m, 14H), 3.72 (dd, J=10.9, 1.9 Hz, 1H), 3.67 (dd, J=10.9, 4.4 Hz, 1H), 3.62 (dd, J=16.6, 8.1 Hz, 1H), 3.58 (dd, J=17.5, 8.2 Hz, 2H), 3.48 (dd, J=9.8, 8.5 Hz, 1H), 3.42 (ddd, J=9.5, 4.4, 2.0 Hz, 1H); ¹³C NMR (126 MHz, CDCl₃) δ 159.53, 159.44, 159.38, 159.31, 158.69, 134.53, 133.62, 130.86, 130.78, 130.73, 130.58, 130.47, 130.42, 130.07, 130.04, 129.69, 129.55, 129.52, 129.47, 129.10, 129.09, 128.92, 127.83, 127.32, 114.03, 114.01, 113.99, 113.97, 113.95, 113.90, 86.76, 83.28, 81.28, 80.38, 77.72, 75.56, 74.99, 74.81, 73.19, 71.62, 68.77, 55.43, 31.89; ⁷⁷Se NMR (95 MHz, CDCl₃) δ 414; IR(neat)/cm⁻¹ 3001, 2905, 2836, 1612, 1513, 1247, 1034; MS (ESI⁺) m/z (rel intensity) 907.18 [100, (M+Ag)⁺]; HRMS (ESI⁺) m/z 907.1511 (907.1509 calcd for C₄₄H₄₈O₉SeAg).

### 2,3,4,6-Tetra-O-p-methoxybenzyl-D-glucopyranoside (15)

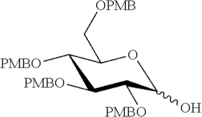

To a solution of the selenoglycoside (14) (5 g, 6.25 mmol) in acetone (I50 mL) was added water (15 mL, 820 mmol). The solution was cooled to 0° C. before the addition of N-bromosuccinimide (2.9 g, 16.4 mmol) and then stirred at room temperature for 2 hours. The solvent was removed in vacuo and the residue partitioned between ethyl acetate (150 mL) and water (150 mL) and the organic layer separated. The aqueous phase was extracted with ethyl acetate (3×50 mL). The combined organic extracts were washed with saturated NaHCO₃ (100 mL), brine (100 mL) and dried over MgSO₄. Evaporation and flash column chromatography eluting with 25-100% ethyl acetate in petroleum ether afforded the protected glycoside (15) as a white solid (3.51 g, 5.31 mmol, 82%). R_{f}0.33 (Hex:EtOAc 1:1); [α]_{D}²²=+0.3° (c 1.0 in CHCl₃) (lit+0.4° c. 1.7 in CHCl₃); ¹H NMR (500 MHz, CDCl₃) δ 7.32-7.20 (m, 14H), 6.93-6.71 (m, 14H), 5.22-5.11 (t, J=2.7 Hz, 1H), 4.85-4.28 (m, 15H), 4.12 (t, J=6.1 Hz, 1H), 4.05 (d, J=6.6 Hz, 1H), 3.98 (dd, J=10.1, 3.7 Hz, 1H), 3.88-3.85 (m, 2H), 3.80-3.75 (m, 22H), 3.75-3.66 (m, 2H), 3.62-3.52 (m, 5H), 3.50 (d, J=7.0 Hz, 1H), 3.35 (dd, J=9.1, 7.7 Hz, 1H); ¹³C NMR (CDCl₃): 6159.42, 159.37, 159.35, 159.29, 159.26, 159.22, 131.01, 130.99, 130.88, 130.80, 130.73, 130.61, 130.09, 130.05, 129.95, 129.78, 129.75, 129.30, 129.26, 113.89, 113.81, 113.72, 113.69, 97.92, 91.99, 82.07, 80.60, 78.62, 76.27, 74.84, 74.37, 74.24, 74.11, 73.66, 73.24, 73.16, 73.13, 72.74, 69.51, 68.94, 68.78, 55.38, 55.36; MS (ESI⁺) m/z (rel intensity) 675.64 [100, (M+15)⁺]; HRMS (ESI⁺) m/z 683.2825 (683.2827 calcd for C₃₈H₄₄O₁₀Na). These data are in good agreement with literature values (L. J. Whalen and R. L. Halcomb, Org. Lett., 2004, 6, 19, 3221-3224).

### 2,3,4,6-Tetra-O-p-methoxybenzyl-1,5-di-O-hydroxy-D-glucitol (16)

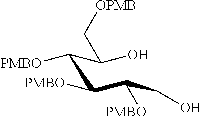

To a solution of the sugar (15) (3 g, 4.5 mmol) in anhydrous methanol (30 mL) under nitrogen at 0° C. was added sodiumborohydride (0.64 g, 17 mmol) portionwise. The solution was then warmed to 50° C. and stirred overnight. The solvent was removed in vacuo and the residue was partitioned between ethyl acetate (100 mL) and water (100 mL) and the organic layer was separated. The aqueous phase was extracted with ethyl acetate (3×50 mL) and the combined organic extracts washed with brine (2×50 mL) and dried over MgSO₄. Evaporation and chromatography (50% ethyl acetate in petroleum ether) afforded the diol (16) as a colourless oil (2.83 g, 4.3 mmol, 95%). R_{f}0.15 (Hex:EtOAc 1:1); [α]_{D}²²=−3.8° (c 0.67 in CHCl₃) (lit −0.5° c. 0.67 in CHCl₃); ¹H NMR (500 MHz, CDCl₃) δ 7.61-6.38 (m, 16H), 4.61-4.36 (m, 8H), 4.02-3.96 (m, 1H), 3.78-3.71 (m, 14H), 3.68-3.64 (m, 2H), 3.50 (dd, J=9.2, 5:8 Hz, 1H), 3.43 (dd, J=6 9, 9.2 Hz), 3.30 (d, J=4.6 Hz, 1H), 2.34 (m, 1H); ¹³C NMR (126 MHz, CDCl₃) δ 159.54, 159.48, 159.46, 159.44, 130.46, 130.29, 130.24, 130.15, 130.12, 129.98, 129.96, 129.75, 129.73, 129.68, 114.04, 114.01, 113.99, 113.96, 113.92, 79.10, 78.90, 74.06, 73.24, 72.96, 72.81, 70.98, 70.91, 62.02, 55.41; IR (neat)/cm⁻¹: 3464, 3002, 2936, 2836, 1612, 1513, 1247, 1033; MS (ESI⁺) m/z (rel intensity) 685.55 [100, (M+Na)⁺]; HRMS (ESI⁺) m/z 685.2983 (685.2983 calcd for C₂₈H₄₆O₁₀Na). These data are in good agreement with literature values (L. J. Whalen and R. L. Halcomb, Org. Lett., 2004, 6, 19, 3221-3224).

### 2,3,4,6-Tetra-O-p-methoxybenzyl-1,5-di-O-methanesulfonyl-D-glucitol (17)

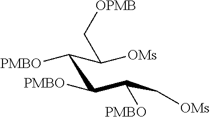

To a stirred solution of the diol (16) (2.5 g, 3.8 mmol), 4-dimethylaminopyridine (DMAP, 38 mg; 0.3 mmol) and anhydrous pyridine (1 mL) in dry CH₂Cl₂ (20 mL) under nitrogen at 0° C. was added dropwise methanesulfonyl chloride (0.9 mL, 11.8 mmol). The solution was stirred at 0° C. for 30 min and then warmed to room temperature for 6 hours. The reaction was quenched by the addition of saturated NaHCO₃ (50 mL) before being extracted with CH₂CL₂ (2×30 mL). The combined organic extracts were then washed with brine (2×50 mL) and dried over MgSO₄. Evaporation afforded the dimesylate (17) as a colourless oil (2.96 g, 3.6 mmol, 95%). The compound was found to decompose readily so it was reacted without further purification. R_{f} 0.42 (Hex:EtOAc 1:1); ¹H NMR (500 MHz, CDCl₃) δ 7.18-7.04 (m, 8H), 6.84-6.73 (m, 8H), 4.91-4.86 (m, 1H), 4.65-4.43 (m, 8H), 4.25-4.10 (m, 2H), 3.98-3.76 (m, 13H), 3.68-3.55 (m, 2H), 2.90 (m, 1H), 2.86 (s, 3H), 2.80 (m, 2H), 2.77 (s, 3H); ¹³C NMR (125 MHz, CDCl₃) δ 159.74, 159.71, 159.67, 159.51, 130.39, 130.24, 130.01, 129.65, 129.49, 129.45, 129.23, 129.18, 114.10, 114.09, 83.48, 78.77, 77.65, 76.54, 73.28, 70.66, 68.98, 56.51, 56.50, 55.50, 54.49: 38.65, 37.25.

### 2,3,4,6-Tetra-O-p-methoxybenzyl-1,5-anhydro-5-seleno-L-iditol (18)

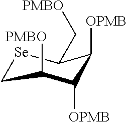

To a stirred suspension of the selenium powder (142 mg, 1.8 mmol) in degassed ethanol (12 mL) under argon at 0° C. was added a saturated solution of sodiumborohydride (˜1 g) in degassed ethanol (3 mL). The suspension was stirred at 0° C. for 10 min and at room temperature for 1 h during which time the black selenium colour disappeared. The clear solution was then cooled to 0° C. for the addition of the dimesylate (17) (1 g, 1.2 mmol) in THF (3 mL). The reaction mixture was heated and stirred at 70° C. for 12 hours. The solvent was removed in vacuo before the residue was partitioned between ethyl acetate (20 mL) and water (20 mL) and the organic layer was separated. The aqueous phase was extracted with ethyl acetate (3×20 mL) and the combined organic extracts were washed with brine (2×20 mL) and dried over MgSO₄. The remaining residue was then dried and dissolved in DMF (5 mL), before the addition of NaBH₄ (100 mg) and refluxed for 24 hours. The solvent was removed in vacuo before the residue was partitioned between ethyl acetate (20 mL) and water (20 mL) and the organic layer was separated. The aqueous phase was extracted with ethyl acetate (3×20 mL) and the combined organic extracts were washed with brine (2×20 mL) and dried over MgSO₄. Evaporation and chromatography (25% ethyl acetate in petroleum ether) afforded the seleno-iditol (18) as a colourless oil (0.20 g, 0.28 mmol, 15%). R_{f}0.29 (Hex:EtOAc 4:1); ¹H NMR (500 MHz, CDCl₃) δ 7.30-7.11 (m, 8H), 6.89-6.74 (m, 8H), 4.74-4.39 (m, 8H), 3.99 (dd, J=7.6, 5.8 Hz, 1H), 3.90-3.73 (m, 12H), 3.56 (dd, J=5.7, 4.5 Hz, 1H), 3.50-3.43 (m, 2H), 3.39 (dd, J=10.0, 5.4 Hz, 1H), 3.21-3.12 (m, 1H), 2.83 (t, J=11.8 Hz, 1H), 2.57 (dd, J=12.3, 4.4 Hz, J_{H,Se}=12.4 Hi, 1H); ¹³C NMR (125 MHz; CDCl₃) δ 159.36, 159.33, 159.20, 159.15, 131.19, 130.57, 130.45, 130.31, 129.71, 129.66, 129.42, 129.40, 129.35, 129.32, 129.14, 129.13, 114.64, 114.79, 113.72, 113.71, 113.68, 83.31′ 82.33, 81.72, 77.27, 77.14, 76.76, 75.66, 72.81, 72.52, 66.75, 54.22, 55.26, 42 00, 27.31; MS (ESI⁺) m/z (rel intensity) 731.58 [100, (M+Na)⁺]; HRMS (ESI⁺) m/z 731.2111 (731.2094 calcd for C₃₈H₄₄O₈SeNa).

### 1,5-Anhydro-5-seleno-L-iditol (19)

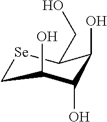

To a stirred solution of the protected seleno sugar (500 mg, 0.71 mmol) in dry CH₂Cl₂ (5 mL) under nitrogen at 0° C. was added TFA (5 mL). The solution was stirred at 0° C. for 10 min and at room temperature for 2 hrs. The solvent was removed in vacuo and the residue was partitioned between CH₂Cl₂ (5 mL) and water (5 mL), the organic phase was extracted with water (2×2 mL). The combined aqueous phases were evaporated giving a brown gum and chromatography (20% methanol in ethyl acetate) afforded the deprotected thio sugar (19) as a white crystalline solid (0.94 g, 0.39 mmol, 55%). R_{f}0.44 (EtOAc:MeOH 5:1); [α]_{D}²²=−83.2° (c 0.1 in MeOH); ¹H NMR (500 MHz, CD₃OD) δ 4.03 (dd, 11.4, 5.5 Hz, 1H), 3.93 (dd, J=8.6, 4.3 Hz, 1H), 3.80 (dd, J=11.4, 7.9 Hz, 1H), 3.71 (ddd, J=9.8, 7.9, 4.0 Hz, 1H), 3.44 (t, J=8.3 Hz, 1H), 3.19-3.11 (m, 1H), 2.75 (dd, J=12.4, 9.8 Hz, 1H), 2.66 (dd, J=12.3, 4.0 Hz, J_{H,Se}=12.4 Hz, 1H); ¹³C NMR (125 MHz, CD₃OD) δ 74.28, 73.64, 73.00, 60.52, 39.99, 20.20; ⁷⁷Se NMR (95 MHz, CD₃0D) δ 76; IR (neat)/cm⁻¹: 3347, 2888, 1420, 1049; MS (ESI⁺) m/z (rel intensity) 251.08 [100, (M+Na)⁺]; HRMS (ESI⁺) m/z 250.9793 (250.9793 calcd for C₁₂H₂₀O₄SeNa); Anal. Calcd for C₆H₁₂O₄Se: C, 31.74; H, 5.33. Found: C, 31.50; H, 5.30.

### Synthesis of 1,5-anhvdro-5-seleno-D-glucitol

### 2,3,4,6-Tetra-O-p-methoxybenzyl-1-tert-butyl-dimethylsilyl-5-O-hydroxy-D-glucitol (20)

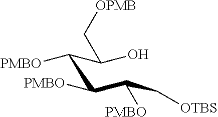

To a solution of the diol (16) (5 g, 7.5 mmol) in DMF (50 mL) under nitrogen at 0° C. was added imidazole (1.28 g, 18.8 mmol) followed by TBDMSCI (1.25 g, ‘8.3 mmol). The solution was stirred at 0° C. for 10 minutes and was then allowed to warm to room temperature and stirred for 2 hours. The reaction mixture was then concentrated in vacuo, poured into water (50 mL), and extracted with ethyl acetate (3×50 mL). The combined organic fractions were washed with saturated NaHCO₃ (2×40 mL), dried over MgSO₄ and concentrated to afford a viscous clear yellow oil. Flash chromatography (25% ethyl acetate in petroleum ether) afforded the silyl ether (20) as a colourless oil (5.55 g, 7.1 mmol, 9%). R_{f}0.39 (Hex:EtOAc 3:1); [α]_{D}²²=−6.1° (c 0.42 in CHCl₃) (Lit −6.9° c. 0.42 in CHCl₃); 1H NMR (500 MHz, CDCl₃) δ 7.30-7.12 (m, 8H), 6.91-6.78 (m, 8H), 4.72-4.39 (m, 8H), 3.99-3.93 (m, 1H), 3.90-3.87 (m, 1H), 3.81-3.79 (m, 12H), 3.78-3.62 (m, 4H), 3.61-3.53 (m, 2H), 0.89 (s, 9H), 0.02 (s, 6H); ¹³C NMR (125 MHz, CDCl₃) δ 159.51, 159.44, 159.41, 130.95, 130.74, 130.64, 130.56, 130.30, 129.87, 129.85, 129.66, 114.00, 113.93, 79.54, 77.95, 73.80, 73.24, 73.10, 73.06, 71.39, 71.27, 63.19, 55.48, 26.16, 18.44, −5.11, −5.15; IR(neat)/cm⁻¹: 3485, 2920, 2853; MS (ESI⁺) m/z (rel intensity) 852.18 [100, (M+75)⁺]; HRMS (ESI⁺) m/z 777.4031 (777.4029 calcd or C₄₄H₆₀O₁₀Si). Anal. Calcd. for C₁₄₄H₆₀O₁₀Si: C, 68.01; H, 7:78; O, 20.59; Si, 3.61. Found: C, 67.93; H, 7.65. These data agree with the published literature values (L. J. Whalen and R. L. Halcomb, Org. Lett., 2004, 6, 19, 3221-3224).

### 2,3,4,6-Tetra-O-p-methoxybenzyl-1-tert-butyl-dimethylsilyl-5-O-hydroxy-L-iditol (21)

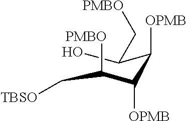

To a solution of the DMSO (I50 μL, 2.2 mmol) in dry CH₂Cl₂ (15 mL) under nitrogen at −78° C. was added oxalyl chloride (140 μL, 1.6 mmol) drop wise. The solution was stirred at −78° C. for 30 minutes before the dropwise addition of the alcohol (5) (420 mg, 0.53 mmol) in CH₂Cl₂ (5 mL). The mixture was stirred for a further hour at −78° C. before the addition of Et₃N (600 μL, 4.3 mmol). After stirring for a further 30 minutes at −78° C. the starting material had disappeared by TLC and the solution was warmed to room temperature. Following dilution with CH₂Cl₂ (50 mL) and addition of saturated NaHCO₃ (50 mL). The organic layer was separated and the aqueous layer was extracted with further dichloromethane (2×50 mL). The combined organic fractions were washed again with saturated H₂O (100 mL), then brine (100 mL) and dried over MgSO₄. Evaporation of the solvent yielded a colourless oil. The oil was dried under vacuum for 2 hours before being dissolved in dry methanol (20 mL) and cooled to −78° C. Anhydrous cerium (III) chloride (200 mg, 0.8 mmol) was added before the portionwise addition of NaBH₄ (20 mg, 0.56 mmol). •The solution was stirred for 10 minutes until TLC showed full consumption of starting material. The solution was then warmed to room temperature and concentrated in vacuo. The remaining residue was diluted with water (75 mL), and extracted with ethyl acetate (3×75 mL). The combined organic fractions were washed with saturated NaCl (2×50 mL), dried over MgSO₄ and concentrated to afford a viscous clear oil as a 4:1 mixture of isomers. Flash chromatography (25% ethyl acetate in petroleum ether) afforded the alcohol (6) as a colourless oil (286 mg, 37 mmol, 68%). R_{f}0.40 (Hex:EtOAc 3:1); [α]_{D}²²=+7.5° (c 1.0 in DCM); ¹H NMR (500 MHz, CDC1₃) δ 7.25-7.12 (m, 8H), 6.93-6.71 (m, 8H), 4.71-4.55 (m, 4H), 4.48-4.33 (m, 4H), 3.89-3.87 (m, 2H), 3.85 (dd, J=7.0, 4.4 Hz, 1H), 3.82-3.76 (m, 12H), 3.68-3.58 (m, 3H), 3.42 (dd, J=9.3, 6.4 Hz, 1H), 3.33 (dd, J=9.4, 6.1 Hz, 1H), 1.26 (s, 9H), 0.07 (s, 6H); ¹³C NMR (126 MHz, CDCl₃) δ 159.15, 130.61, 130.59, 130.51, 130.20, I29.99, 129.82, 129.79, I29.56, 129.37, 129.31, 113.69, 113.61, 78.63, 78.33, 78.06, 74.24, 74.19, 72.81, 72; 68, 71.15, 69.65, 63.27, 55.I7, 25.86, 18.11, −5.11, −5.15; IR (neat)/cm⁻¹: 3490, 2930, 1612, 1514, 1248, 1035; MS (ESI⁺) m/z (rel intensity) 799.58 [100, (M+Na)⁺]; HRMS (ESI⁺) m/z 799.3848 (799.3848 calcd for C₄₄H₆₀O₁₀SiNa); Anal. Calcd. for C₄₄H₆₀O₁₀Si: C, 68.01; H, 7.78. Found: C, 67.95; H, 7.65.

### 2,3,4,6-tetra-O-p-methoxybenzyl-1,5-di-O-hydroxy-L-iditol (22)

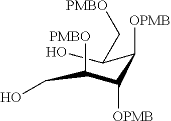

To a stirred solution of (22) (100 mg, 0.13 mmol) in dry THF (50 mL) under nitrogen•at room temperature was added TBAF (0.15 mL of a 1.0M solution in THF, 0.15 mmol) dropwise. After 1 hour the mixture was diluted with ethyl acetate (200 mL) and washed with water (2×100 mL) followed by brine (100 mL). Drying over MgSO₄ and concentration in vacuo afforded compound (23) as a clear colourless oil (77 mg, 0.12 mmol, 90%). R_{f}0.I9 (Hex:EtOAc 1:1); [α]_{D}²²=+5.0° (c 1.0 in DCM); ¹H NMR (500 MHz, CDCl₃) δ 7.18 (dd, J=14.0, 8.6 Hz, 8H), 6.91-6.85 (m, 8H), 4.49 (tt, J=11.7, 7.9 Hz, 8H), 4.32 (m, 2H), 4.21 (q, J=5.0 Hz, 1H), 4.05 (m, 2H), 3.84-3.79 (m, 12H), 3.75 (dd, J=11.9, 4.9 Hz, 1H), 3.66 (m, 2H); ¹³C NMR (125 MHz, CDCl₃) δ 159.49, 159.39, 159.20, 159.19, 130.31, 129.90, 129.44, 129.29, 113.99, 113.86, 113.74, 113.72, 82.52, 81.27, 79.54, 78.91, 73.14, 72.06, 71.87, 68.02, 61.95, 55.28, 26.15, 18.47; IR (neat)/cm⁻¹: 3449, 2932, 1612, 1514, 1249, 1034; MS (ESI⁺) m/z (rel intensity) 685.67 [100, (M+Na)⁺]; HRMS (ESI⁺) m/z 685.2980 (685.2983 calcd for C₃₈H₄₆O₁₀Na).

### 2,3,4,6 tetra-O-p-methoxybenzyl-1,5-di-O-methansulfonyl-L-iditol (23)

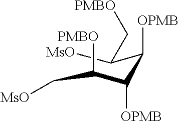

To a stirred solution of the diol (23) (100 mg, 0.15 mmol), 4-dimethylaminopyridine (DMAP, 4 mg, 0.03 mmol) and anhydrous pyridine (1 mL) in dry CH₂Cl₂ (15 mL) under nitrogen at 0° C. was added dropwise methanesulfonyl chloride (68 μL, 0.9 mmol). The solution was stirred at 0° C. for 30 min and then warmed to room temperature for 6 hours. The reaction was quenched by the addition of saturated NaHCO₃ (50 mL) before being extracted with CH₂Cl₂ (2×30 mL). The combined organic extracts were then washed with brine (2×50 mL) and dried over MgSO₄. Evaporation and chromatography (25% ethyl acetate in hexane) afforded the dimesylate (24) as a colourless oil (118 g, 0.14 mmol, 95%). R_{/}0.55 (Hex:EtOAc 1:1); [α]_{D}²²=−7.7° (c 1.0 in DCM); ¹H NMR (500 MHz, CDCl₃) δ 7.17-7.06 (m, 8H), 6.83-6.73 (m, 8H), 4.89-4.S5 (m, 1H), 4.65-4.43 (m, 8H), 4.21-4.10 (m, 2H), 3.95-3.89 (m, 1H), 3.84-3.76 (m, 12H), 3.68-3.61 (m, 1H), 3.59-3.54 (m, 1H), 2.88 (dd, J=6.0, 3.0 Hz, 1H), 2.87 (s, 3H), 2.81 (dd, J=9.5, 3.0 Hz, 2H), 2.78 (s, 3H); ¹³C NMR (126 MHz, CDCl₃) δ 159.74, 159.71, 159.67, 159.51, 133.54, 131.13, 131.05, 130.45, 129.65, 129.45, 129.18, 127.14, 114.27, 114.10, 81.48, 76.40, 75.04, 74.15, 73.03, 72.15, 69.17, 6.43, 56.38, 52.69, 38.67, 37.27; IR (neat)/cm⁻¹: 2960, 1612, 1514, 1253, 1174, 1034.

### 2,3,4,6 tetra-O-p-methoxybenzyl-1,5-anhydro-5-seleno-D-glucitol (25)

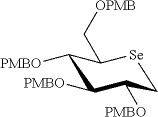

To a stirred suspension of the selenium powder (I42 mg, 1.8 mmol) in degassed ethanol (12 mL) under argon at 0° C. was added a saturated solution of sodiumborohydride (˜1 g) in degassed ethanol (3 mL). The suspension was stirred at 0° C. for 10 min and at room temperature for 1 h during which time the black selenium colour disappeared. The clear solution was then cooled to 0° C. for the addition of the dimesylate (24) (1 g, 1.2 mmol) in THF (3 mL). The reaction mixture was heated and stirred at 70° C. for 12 hours. The solvent was removed in vacuo before the residue was partitioned between ethyl acetate (20 mL) and water (20 mL) and the organic layer was separated. The aqueous phase was extracted with ethyl acetate (3×20 mL) and the combined organic extracts were washed with brine (2×20 mL) and dried over MgSO₄. The remaining residue was then dried and dissolved in DMF (5 mL), before the addition of NaBH₄ (100 mg) and refluxed for 24 hours. The solvent was removed in vacuo before the residue was partitioned between ethyl acetate (20 mL) and water (20 mL) and the organic layer was separated. The aqueous phase was extracted with ethyl acetate (3×20 mL) and the combined organic extracts were washed with brine (2×20 mL) and dried over MgSO₄. Evaporation and chromatography (25% ethyl acetate in petroleum ether) afforded the seleno-glucitol (25) as a colourless oil (0.26 g, 0.36 mmol, 20%). R_{f}0.52 (Hex:EtOAc 3:1); ¹H NMR (500 MHz, CDCl₃) δ 7.28-7.22 (m, 8H), 6.91-6.79 (m, 8H), 4.88-4.58 (m, 4H), 4.55-4.38 (m, 4H), 4.33 (dd, J=11.4, 3.2 Hz, 1H), 3.94-3.85 (m, 1H), 3.83-3.79 (m, 12H), 3.78-3.71 (m, 1H), 3.31 (t, J=9.0 Hz, 1H), 3.19 (dt, J=9.1, 4.7 Hz, 1H), 2.77 (t, J=7.0 Hz, 1H), 2.70 (dd, J=12.5, 4.2 Hz, 1H), 2.63 (t, J=11.7 Hz, J_{H,se}=12.4 Hz, 1H); ¹³C NMR (125 MHz, CDCl₃) δ 160.23, 159.46, 159.34, 159.85, 131.32, 131.04, 130.76, 130.09, 129.67, 129.61, 129.55, 129.42, 114.16, 114.01, 113.88, 113.73, 86.22, 84.24, 80.87, 74.97, 73.23, 72.10, 70.13, 68.11, 57.38, 55.17, 54.25, 47.38, 30.43; MS (ESI⁺) m/z (rel intensity) 731.21 [100, (M+Na)⁺]; HRMS (ESI⁺) m/z 731.5132 (731.5133 calcd for C₃₈H₄₄O₈SeNa).

### 1,5-anhydro-5-seleno-D-glucitol (26)

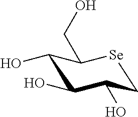

To a stirred solution of the protected seleno sugar (25) (500 mg, 0.71 mmol) in dry CH₂Cl₂ (5 mL) under nitrogen at 0° C. was added TFA (5 mL). The solution was stirred at 0° C. for 10 min and at room temperature for 2 hrs. The solvent was removed in vacuo and the residue was partitioned between CH₂Cl₂ (5 mL) and water (5 mL), the organic phase was extracted with water (×2 mL). The combined aqueous phases were evaporated giving a brown gum and chromatography (20% methanol in ethyl acetate) afforded the deprotected seleno sugar (26) as a white crystalline solid (0.94 g, 0.39 mmol, 55%). R_{f}0.40 (EtOAc:MeOH 5:1); [α]_{D}²²=+15.6° (c 0.1 in MeOH); ¹H NMR (500 MHz, CD₃OD) δ 4.18 (m, 1H), 4.00 (dd; J=11.4, 6.8 Hz, 1H), 3.96 (t, J=9.0 Hz, 1H), 3.77 (dd, J=15.4, 8.9 Hz, 1H), 3.37 (dd, J=15.4, 5.0 Hz, IH), 3.09-3.05 (m, IH), 2.86. (dd, J=5.0, 8.9 Hz, 1H), 2.70 (dd, J=12.1, 8.9 Hz, J_{H,Se}=12.4 Hz, 1H); ¹³C NMR (125 MHz, CD₃OD) δ 79.38; 75.42, 73.96, 62.09, 42.73, 22.18; ⁷⁷Se NMR (95 MHz, CD₃OD) δ 133.50; MS (ESI⁺) m/z (rel intensity) 251.08 [100, (M+Na)⁺]; HRMS (ESI⁺) m/z 250.9793 (250.9793 calcd for C₁₂H₂₂O₄SeNa); Anal. Calcd. for C₆H₁₂O₄Se: C, 31.74; H, 5.33. Found: C, 31.41; H, 5.32.

### Alternative synthesis of 1,5-anhydro-5-seleno-L-iditol

### 1-Benzyl-2,3,4,6-Di-O-isopropylidene-D-glucopyranoside (27)

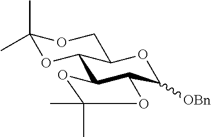

To a suspension of D-glucose (10 g, 55.5 mmol) in benzyl alcohol (80 mL) containing acetyl chloride (5 mL, 70 mmol) was heated at 60° C. for 4 days. The solution was then concentrated in vacuo to a viscous yellow oil, excess benzyl alcohol was azeotropically distilled off by addition of water. The oil was then dried via azeotropic distillation with toluene to give the benzyl ether as a clear oil (11.99 g, 44.4 mmol) that was •reacted without further purification. The oil was then dissolved in dry acetone (250 mL) with p-toluenesulfonic acid monohydrate (200 mg, 1.1 mmol) over 4 Å molecular sieves before the dropwise addition of 2-methoxypropene (10.6 mL, 8.0 g, 222 mmol) at ˜5-10° C. The solution was allowed to warm to room temperature and stirred overnight. The resulting pale yellow solution was quenched by the addition of NaCO₃ (5 g), then filtered and•the•solvent was removed in vacuo to give a yellow oil. The residue was partitioned between EtOAc (200 mL) and water (200 mL) and the organic layer was separated. The aqueous phase was extracted with EtOAc (2×100 mL) and the combined organic extracts washed with brine (2×80 mL) and dried over MgSO₄. Evaporation of the solvent and chromatography (25%-67% EtOAc in Pet.) afforded the protected sugar (27) as a white amorphous solid (15.6 g, 44.4 mmol, 80% over 2 steps). R_{f}0.39 (Hex:EtOAc 6:1); [α]_{D}²²=+33.1° (c 1.0 in CHCl₃) (Lit+35° c. 1.0 in CHCl₃); ¹H NMR (500 MHz, CDCl₃) δ 7.24-7.38 (m, 5H), 5.26 (d, J 2.6 Hz, 1H), 4.79 (d J=12.4 Hz, 1H), 4.67 (d, J=12.4 Hz, 1H), 4.12 (t, J=9.2 Hz, 1H), 3.91 (t, J=9.5 Hz, 1H), 3.83 (m, 2H), 3.37 (m, 2H), 1.55 (s, 3H), 1.50 (s, 3H), 1.47 (s, 3H), 1.44 (s, 3H); ¹³C NMR (125 MHz, CDCl₃) δ 137.3, 128.4, 127.8, 127.6, 111.5, 99.7, 97.4, 76.9, 73.9, 73.8, 69.9, 65.2, 62.3, 28.9, 26.9, 26.4, 19.2; MS (ESI⁺) m/z (rel intensity) 333.42 [100, (M-17)+]; HRMS (ESI⁺) m/z 373.1621. (373.1622 calcd for C₁₉H₂₆O₆Na); Anal. Calcd. for C₁₉H₂₆O₆: C, 65.13; H, 7.48. Found: C, 65.15; H, 7.51. These data agree with the published literature values (A. M. Gomez, G. O. Danelo, S. Valverde and J. C. Lopez, Carb. Res., 1999, 320, 1-2, 138-142).

### 2,3,4,6-Di-O-isopropylidene-1,5,-di-O-hydroxy-D-glucitol (28)

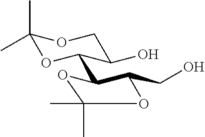

The protected sugar (27)•(10 g, 28.6 mmol) was dissolved in EtOH (50 mL) and Et₃N (5 mL and hydrogenated in a Parr hydrogenator with 10% Pd/C (2 g, 20% w/w) at 50 psi for 24 hours until all•the starting material had been consumed. The solution was filtered through celite before the portionwise addition of NaBH₄ (1.0 g, 26.6 mmol). The solution was stirred at room temperature for 3 hours to reduce any unreacted sugar. The solvent was removed in vacuo and the residue was partitioned between EtOAc (150 mL) and water (150 mL) and the organic layer was separated. The aqueous phase was extracted with EtOAc (4×50 mL) and the combined organic extracts washed with brine (2×50 mL) and dried over MgSO₄. Evaporation and chromatography (25%-67% EtOAc in Pet.) afforded the dial (28) as a colourless oil (6.74 g, 25.74 mmol, 90% over 2 steps). R_{f}0.13 (Hex:EtOAc 1:1); [α]_{D}²²=−25.6° (c 1.0 in DCM) (Lit-17.3° c. 0.8 in CHCl₃); ¹H NMR (500 MHz, CDCl₃) δ 4.23 (dt, J=8.7, 4.4 Hz, 1H), 4.15 (dd, J=8.2, 3.8 Hz, 1H), 3.93-3.79 (m, 3H), 3.78-3.73 (m, 2H), 3.67 (dd, J=11.8, 4.3 Hz, 1H), 3.64-3.58 (m, 1H), 1.44 (s, 3H), 1.41 (s, 3H), 1.40 (s, 3H), 1.36 (s, 3H); ¹³C NMR (125 MHz, CDCl₃) δ 109.47, 99.02, 77.28, 77.17, 77.03, 76.78, 76.54, 72.07, 64.12, 63.48, 62.67, 28.07, 27.04, 26.57, 19.32; IR(neat)/cm⁻¹: 3433, 2986, 1217, 1066; MS (ESIS⁺) m/z (rel intensity) 280.25 (100, (M+18)⁺]; HRMS (ESI⁺) m/z 285.1308 (285.1309 calcd for C₁₂H₂₂O₆Na); Anal. Calcd. for C₁₂H₂₂O₆: C, 54.95; H, 8.45. Found: C, 55.02; H, 8.36. These data agree with the published literature values (A. M. Gomez, G. O. Danelo, S. Valverde and J. C. Lopez, Carb. Res., 1999, 320, 1-2, 138-142).

### 2,3,4,6-Di-O-isopropylidene-1,5-di-O-methanesulfonyl-D-glucitol (29)

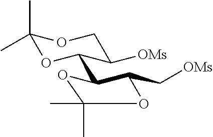

To a stirred solution of the dial (28) (5 g, 19 mmol), DMAP (250 mg, 2 mmol) and anhydrous pyridine (10 mL) in dry DCM (150 mL) under nitrogen at 0° C., was added dropwise methanesulfonyl chloride (4.5 mL, 59 mmol). The solution was stirred at 0° C. for 30 minutes and then warmed to room temperature for 6 hours. The reaction was quenched by the addition of saturated NaHCO₃ (50 mL) before being extracted with DCM (3×50 mL). The combined organic extracts were then washed with brine (2×100 mL) and dried over MgSO₄. Evaporation and chromatography (Hex:EtOAc 1:1) afforded the dimesylate (29) as a white amorphous solid (7.07 g, 16.8 mmol, 89%). R_{f}0.28 (Hex:EtOAc 1:1); ¹H NMR (500 MHz, CDCl₃) δ 4.81 (ddd, J=8.9, 7.1, 5.1 Hz, 1H), 4.42 (dt, J=7.7, 4.7 Hz, 1H), 4.34 (m, 2H), 4.14 (ddd, J=8.7, 6.8, 5.0 Hz, 3H), 3.90 (dd, J=12.1, 7.2 Hz, 1H), 3.81 (dd, J=8.9, 2.2 Hz, 1H), 3.10-3.09 (m, 3H), 3.09-3.08 (m, 3H), 1.47 (s, 3H), 1.44 (s, 4H), 1.43 (s, 3H), 1.41 (s, 3H); ¹³C NMR (125 MHz, CDCl₃) δ 110.94, 100.05, 77.25, 77.00, 76.74, 75.49, 72.97, 72.81, 68.65, 68.39, 62.56, 37.84, 37.74, 27.26, 26.89, 26.37, 20.21; MS (ESI⁺) m/z (rel intensity) 441.18 [100, (M+Na)⁺]; HRMS (ESI⁺) m/z 441.0860 (441.0859 calcd for C₁₄H₂₆O₁₀S₂Na).

### 2,3,4,6-Di-O-isopropylidene-1,5-anhydro-5-seleno-L-iditol (30)

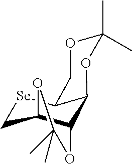

To a stirred suspension of selenium powder (0.85 g, 10.8 mmol) in degassed EtOH (40 mL) under argon at 0° C. was added a saturated solution of NaBH₄ (˜1 g) in degassed EtOH (10 mL). The suspension was stirred at 0° C. for 10 minutes and at room temperature for 1 hour during which time the black selenium suspension disappeared. The clear solution was then cooled to 0° C. for the addition of the dimesylate (29) (3 g, 7.2 mmol) in THF (5 mL). The reaction mixture was heated and stirred at 70° C. for 12 hours. The solvent was removed in vacuo before the residue was partitioned between EtOAc (50 mL) and water (50 mL) and the organic layer was separated. The aqueous phase was extracted with EtOAc (3×30 mL) and the combined organic extracts were washed with brine (2×30 mL) and dried over MgSO₄. Evaporation and chromatography (Hex:EtOAc 4:1) afforded the seleno-gulitol (30) as a white amorphous solid (1.28 g, 4.25 mmol, 59%). R_{f}0.38 (Hex:EtOAc 4:1); [α]_{D}²²=−9.2° (c 1.0 in DCM); ¹H NMR (500 MHz, CDCl₃) δ 4.32 (dd, J=7.7, 5.5 Hz, 1H), 4.01-3.89 (m, 2H), 3.90-3.79 (m, 2H), 3.76-3.67 (m, 1H), 3.10 (dd, J=9.7, 5.2 Hz, 1H), 2.80 (dd, J=10.8, 9.7 Hz, J_{H,Se}=12.4 Hz, 1H), 1.48 (s, 3H), 1.46 (s, 3H), 1.44 (s, 3H), 1.43 (s, 3H); ¹³C NMR (MHz; CDCl₃) δ 110.22, 99.37, 82.28, 77.25, 76.99, 76.74, 74.65, 73.30, 61.64, 31.78, 27.22, 27.02, 25.83, 25.00, 20.26; ⁷⁷Se NMR (95 MHz; CDCl₃) δ 129; MS (ESI⁺) m/z (rel intensity) 189.25•[100, (M-118)⁺]; IR (neat)/cm⁻¹: 2986, 2927, 1371, 1226; 1069; HRMS (ESI⁺) m/z 414.9576 (414.9572 calcd for C₁₂H₂₀O₄SeAg); Anal. Calcd. for C₆H₁₂O₄Se: C, 46.91; H, 6.56. Found: C, 46.83; H, 6.43.

### 1,5-Anhydro-5-seleno-L-iditol (19)

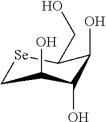

To a stirred solution of the protected seleno-sugar 30 (100 mg, 0.14 mmol) in dry DCM (10 mL) under nitrogen at 0° C. was added TFA (1 mL). The solution was stirred at 0° C. for 10 minutes and at room temperature for 3 hours. The solvent was removed in vacuo and the residue was purified by chromatography (EtOAc:MeOH 4:1) to afford the deprotected seleno-sugar (19) as a colourless oil (18 mg, 0.08 mmol, 59%). R_{f}0.44 (EtOAc:MeOH 5:1); [α]_{D}²²=−83.2° (c 0.1 in MeOH); ¹H NMR (500 MHz, CD₃OD) δ 4.03 (dd, J=11.4, 5.5 Hz, 1H), 3.93 (dd, J=8.6, 4.3 Hz, 1H), 3.80 (dd, J=11.4, 7.9 Hz, 1H), 3.7 (ddd, J=9.8, 7.9, 4.0 Hz, 1H), 3.44 (t, J=8.3 Hz, 1H), 3.19-3.11 (m, 1H), 2.75 (dd, J=12.4, 9.8 Hz, 1H), 2.66 (dd, J=12.3, 4.0 Hz, J_{H,Se}=12.4 Hz, 1H); ¹³C NMR (125 MHz, CD₃OD) δ 74.28, 73.64, 73.00, 60.52, 39.99, 20.20; ⁷⁷Se NMR (95 MHz, CD3OD) δ 76; IR (neat)/cm⁻¹: 3347, 2888, 1420, 1049; MS (ESI⁺) m/z (rel intensity) 251.08 [100, (M+Na)⁺]; HRMS (ESI⁺) m/z 250.9793 (250.9793 calcd for C₁₂H₂₀O₄SeNa); Anal. Calcd. for C₆H₁₂O₄Se: C, 31.74; H, 5.33. Found: C, 31.50; H, 5.30.

### Synthesis of 1,5-anhvdro-5-seleno-D-glucitol

### 2,3,4,6-Di-O-isopropylidene-1-tert-butyl-dimethylsilyl-5-O-hydroxy-D-glucitol (31)

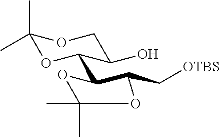

To a solution of the dial (28) (5 g, 19.1 mmol) in dry DCM (100 mL) under nitrogen at 0° C. was added imidazole (3.2 g, 47.7 mmol) followed by TBDMSCI (3.16 g, 21.0 mmol). The solution was stirred at 0° C. for 10 minutes and was then allowed to warm to room temperature and stirred for 3 hours, during which time a solid white precipitate formed. The reaction mixture was then diluted with DCM (100 mL) and poured into water (100 mL). The organic fraction was washed with saturated NaHC0₃ (2×40 mL), dried over MgSO₄ and concentrated to afford a viscous clear yellow oil. Flash chromatography (Hex:EtOAc 5:1) afforded the silyl ether (31) as a colourless oil (6.68 g, 17.7 mmol, 93%). R_{f}0.40 (Hex:EtOAc 5:1); [α]_{D}²²=+11.0° (c 1.0 in DCM); ¹H NMR (500 MHz, CDCl₃) δ 4.18 (dd, J=8.1, 1.8 Hz, 1H), 4.15-4.05 (m, 2H), 4.05-3.98 (m, 2H), 3.82 (dd, J=10.7, 4.0 Hz, 1H), 3.74 (dd, J=10.7, 5.7 Hz, 1H), 3.52 (ddd, J=11.0, 7.4, 2.1 Hz, 1H), 1.42 (s, 3H), 1.41 (s, 3H), 1.40 (s, 3H), 1.35 (s, 3H), 0.90 (s, 9H), 0.07 (s, 6H); ¹³C NMR (126 MHz, CDCl₃) δ 109.50, 109.49, 78.08, 77.11, 76.56, 71.00, 67.36, 63.54, 27.30, 27.17, 26.95, 26.04, 25.46, 18.47, −5.27, −5.36; IR (neat)/cm⁻¹: 3491, 2987, 2931, 1371, 1253, 1068; MS (ESI⁺) m/z (rel intensity) 337.33 [100, (M-39)⁺]; HRMS (ESI⁺) m/z 399.2174 (399.2173 calcd for C₁₈H₃₆O₆SiNa).

### 2,3,4,6-Di-O-isopropylidene-1-tert-butyl-dimethylsilyl-5-O-hydroxy-L-iditol (32)

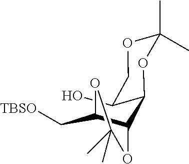

To a solution of DMSO (3.72 mL, 54.1 mmol) in dry DCM (100 mL) under nitrogen at −78° C. was added oxalyl chloride (3.43 mL, 39.4 mmol) dropwise, maintaining the temperature. After stirring for 30 minutes the alcohol (31) (5.0 g, 13.3 mmol) in DCM (25 mL) was added dropwise. After stirring for 1 hour Et₃N (14.68 mL, 105 mmol) was added dropwise and the solution was stirred for an additional hour at −78° C. before slowly being warmed to room temperature. The reaction mixture was then diluted with DCM (100 mL) and poured into water (100 mL). The organic fraction was washed with saturated NaHCO₃ (2×50 mL) and brine (50 mL), dried over MgSO₄ and concentrated to afford a viscous clear yellow oil. Flash chromatography (Hex:EtOAc 10:1) afforded the ketone as a colourless oil (4.38 g, 11.71 mmol, 88%). To a solution of the ketone (1 g, 2.6 mmol) in dry MeOH (50 mL) at −78° C. was added CeCl₃7H₂O (1.07 g, 2.86 mmlol). The solution was stirred for 5 minutes before the portionwise addition of NaBH₄ (152 mg, 4 mmol). The solution was then allowed to warm to room temperature before being filtered through celite and the solvent removed in vacuo. The remaining residue was dissolved in EtOAc (100 mL) and water (75 mL). The organic layer was then separated and the aqueous layer was extracted with EtOAc (50 mL). The combined organic fractions were washed with saturated NaHCO₃ (75 mL), then brine (75 mL) and dried over MgSO₄. Evaporation of the solvent afforded a viscous clear oil. Flash chromatography (Hex:EtOAc 4:1) afforded the alcohol (32) as a colourless oil (881 mg, 2.34 mmol, 90%). R_{f}0:18 (Hex:EtOAc 5:1); [α]_{D}²²=−1.3° (c 1.0 in DCM); ¹H NMR (500 MHz, CDCl₃) δ 4.10-3.99 (m, 2H), 3.95-3.80 (m, 4H), 3.67 (dd, J=11.1, 5.5 Hz, 1H), 3.63 (dd, J=10.0 Hz, 2.3 Hz, 1H), 1.45 (s, 3H), 1.42 (s, 3H), 1.40 (m, 6H), 0.91 (d, 9H), 0.08 (s, 6H); ¹³C NMR (126 MHz, CDCl₃) 109.70, 98.93, 81.57, 74.37, 74.16, 65.63, 64.50, 63.32, 29.37, 27.34, 27.17, 26.04, 25.91, 25.70, −5.1, −5.26; IR (neat)/cm⁻¹: 3491, 2987, 2930, 1370, 1252, 1068; MS (ESI⁺) m/z (rel intensity) 377 [100, (M+H)⁺]; HRMS (ESI⁺) m/z 377.2354 (377.2354 calcd for C₁₈H₃₆O₆Si+H).

### 2,3,4,6-Di-O-isopropylidene-1,5-di-O-hydroxy-L-iditol (33)

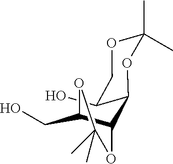

To a stirred solution of (32) (1.0 g, 2.7 mmol) in dry THF (20 mL) under nitrogen at room temperature was added TBAF (2.97 mL of a 1.0 M solution in THF, 2.97 mmol) dropwise. After 1 hour the mixture was diluted with EtOAc (100 mL) and washed with water (2×100 mL) followed by brine (100 mL). Drying over MgSO₄ and concentration in vacuo afforded compound (33) as a colourless oil (0.63 g, 2.40 mmol, 89%). R_{f} 0.19 (Hex:EtOAc 1:1); ¹H NMR (500 MHz, CDCl₃) δ 4.23 (dd, J=13.5, 6.8 Hz, 1H), 4.20-4.13 (m, 1H), 4.12-4.04 (m, 2H), 3.97-3.91 (m, 1H), 3.89-3.83 (m, 1H), 3.81 (d, J=11.6 Hz, 1H), 0.3.63 (dd, J=9.0, 5.7 Hz, 1H), 1.44 (s, 3H), 1.43 (s, 3H), 1.42 (s, 3H), 1.38 (s, 3H); ¹³C NMR (126 MHz, CDCl₃) δ 109.79, 109.66, 77.97, 77.47, 76.39, 70.61, 66.26, 62.27, 27.24, 27.01, 26.66, 25.54; IR (neat)/cm⁻¹: 3435, 2981, 1217, 1064; MS (ESI⁺) m/z (rel intensity) 285.42 [100, (M+Na)⁺]; HRMS (ESI⁺) m/z 285.1309 (285.1309 calcd for C₁₂H₂₂O₆Na); Anal. Calcd. for C₁₂H₂₂O₆: C, 54.95; H, 8.45; O, 36.60. Found: C, 55.17; H, 8.38.

### 2,3,4,6-Di-O-isopropylidene-1,5-di-O-methanesulfonyl-L-iditol (34)

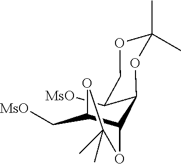

To a stirred solution of the diol (33) (1 g, 3.8 mmol), DMAP (50 mg, 0.4 mmol) and anhydrous pyridine (5 mL) in dry DCM (50 mL) under nitrogen at 0° C. was added dropwise methanesulfonyl chloride (0.9 mL, 11.8 mmol). The solution was stirred at 0° C. for 30 minutes and was then warmed to room temperature and stirred for an additional 3 hours. The reaction was quenched by the addition of saturated NaHCO₃ (20 mL) before being extracted with DCM (3×20 mL). The combined organic extracts were then washed with brine (2×50 mL) and dried over MgSO₄. Evaporation and chromatography (Hex:EtOAc 1:1) afforded the bis-mesylate (34) as a white amorphous solid (1.51 g, 3.61 mmol, 95%). R_{f}0.42 (Hex:EtOAc 2:1); ¹H NMR (500 MHz, CDCl₃) δ 4.67 (dd, J=3.8, 1.9 Hz, 1H), 4.45 (dd, J=11.1, 3.0 Hz, 1H), 4.43-4.38 (m, 1H), 4.30 (dd, J=11.1, 5.6 Hz, 1H), 4.24 (dd, J=13.8, 2.3 Hz, 1H), 4.15 (dd, J=6.4, 1.6 Hz, 1H), 4.11 (dd, J=7.3, 5.8 Hz, 1H), 4.06 (dd, J=13.8, 1.8 Hz, 1H), 3.16 (s, 3H), 3.07 (s, 3H), 1.48 (s, 3H), 1.47 (s, 3H), 1.47 (s, 3H), 1.44 (s, 3H); ¹³C NMR (I26 MHz, CDCl₃) δ 110.91, 99.77, 76.02, 75.23, 71.58, 70.37, 69.58, 62.60, 39.57, 37.65, 28.66, 27.24, 27.18, 18.72; IR (neat)/cm⁻¹: 2968, 1730, 1366, 1217; MS (ESI⁺) m/z (rel intensity) 441.18 [100, (M+Na)⁺]; HRMS (ESI⁺) m/z 441.0859 (441.0859 calcd for C₁₄H₂₆O₁₀S₂Na); Anal. Calcd: for C₁₄H₂₆O₁₀S₂: C, 40.18; H, 6.26. Found: C, 40.21; H, 6.15.

### 2,3,4,6-Di-O-isopropylidene-1,5-anhydro-5-seleno-D-glucitol (35)

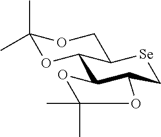

To a stirred suspension of selenium powder (100 mg, 1.3 mmol) in degassed EtOH (8 mL) under argon at 0° C. was added a saturated solution of NaBH₄ (˜100 mg) in degassed EtOH (2 mL). The suspension was stirred at 0° C. for 10 minutes and at room temperature for 10 minutes during which time the black selenium colour disappeared. The clear solution was then cooled to 0° C. for the addition of the bis-mesylate (34) (300 mg, 0.72 mmol) in THF (2 mL). The reaction mixture was heated and stirred at 70° C. for 12 hours. The solvent was removed in vacuo and the residue was partitioned between EtOAc (20 mL) and water (20 mL) and the organic layer was separated. The aqueous phase was extracted with EtOAc (3×10 mL) and the combined organic extracts were washed with brine (2×20 mL) and dried over MgSO₄. Evaporation and chromatography (Hex:EtOAc 4:1) afforded the seleno-gulitol (35) as a white amorphous solid (132 mg, 0.43 mmol, 60%). R_{f}0.51 (Hex:EtOAc 3:1); ¹H NMR (5.00 MHz, CDCl₃) δ 4.36 (dt, J=8.0, 6.7 Hz, 1H), 4.11 (m, 1H), 4.02 (dd, J=11.6, 5.5 Hz, 1H), 3.99-3.92 (m, 1H), 3.89 (dd, J=11.7, 8.2 Hz, 1H), 3.33-3.25 (m, 1H), 2.96 (t, J=11.2 Hz, 1H), 2.85 (dd, J=11.1, 3.7 Hz, J_{H,Se}=12.4 Hz, 1H), 1.46 (s, 3H), 1.44 (s, 3H), 1.43 (s, 3H), 1.40 (s, 3H); ¹³C NMR (126 MHz, CDCl₃) δ 109.79, 99.85, 78.50, 75.77, 74.88, 62.31, 42.56, 35.87, 29.76, 27.09, 22.57, 19.34; ⁷⁷Se NMR (95 MHz; CDCl₃) δ 876; MS (ESI⁺) m/z (rel intensity) 408.17 [100, (M+101)⁺]; HRMS (ESI⁺) m/z 416.9572 (416.9569 calcd for C₁₂H₂₀O₄SeAg); Anal. Calcd. for C₆H₁₂O₄Se: C, 46.91; H, 6.56. Found: C, 46.89; H, 6.50.

### 1,5-anhydro-5-seleno-D-glucitol (26)

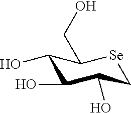

To a stirred solution of the protected seleno sugar (35) (100 mg, 0.14 mmol) in dry DCM (10 mL) under nitrogen at 0° C. was added TFA (1 mL). The solution was stirred at 0° C. for 10 minutes and at room temperature for 3 hours. The solvent was removed in vacuo and the residue was purified by chromatography (EtOAc:MeOH 4:1) to afford the deprotected seleno-sugar 26 as a colourless oil (18 mg, 0.08 mmol, 59%). Rf 0.40 (EtOAc:MeOH 5:1); [α]_{D}²²=+15.6° (c 0.1 in MeOH); ¹H NMR (500 MHz, CD₃OD) δ 4.18 (m, 1H), 4.00 (dd, J=11.4, 6.8 Hz, 1H), 3.96 (t, J=9.0 Hz, 1H), 3.77 (dd, J=15.4, 8.9 Hz, 1H), 3.37 (dd, J=15.4, 5.0 Hz, 1H), 3.09-3.05 (m, 1H), 2.86 (dd, J=5.0, 8.9 Hz, 1H), 2.70 (dd, J=12.1, 8.9 Hz, J_{H,Se}=12.4 Hz, 1H); ¹³C NMR (125 MHz, CD₃OD) δ 79.38, 75.42, 73.96, 62.09, 42.73, 22.18; ⁷⁷Se NMR (95 MHz, CD₃OD) δ 133.50; MS (ESI⁺) m/z (rel intensity) 251.08 [100, (M+Na)⁺]; HRMS (ESI⁺) m/z 250.9793 (250.9793 calcd for C₁₂H₂₂O₄SeNa); Anal. Calcd. for C₆H₁₂O₄Se: C, 31.74; H, 5.33. Found: C, 31.41; H, 5.32.

### Synthesis of 1,4-anhydro-4-seleno-I-talitol

### 2,3,5,6-Di-isopropylidene-1,4,-di-O-hydroxy-D-mannitol (36)

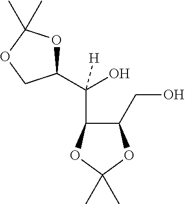 [SMILES: CC1(C)OC[C@H]([C@@H](O)[C@@H]2OC(C)(C)O[C@@H]2CO)O1]

To a suspension of D-mannose (10 g, 55.5 mmol) and p-toluenesulfonic acid monohydrate (1.06 g, 555 mmol) in acetone (200 mL) at 0° C. was added 2,2-imethoxypropane (50 mL) dropwise over 30 minutes. The suspension was allowed to warm to room temperature and stirred overnight. The resulting pale yellow solution was quenched by the addition of NaCO₃ (2 g). Filtration and removal of the solvent in vacuo gave a yellow oil. The residue was partitioned between ethyl acetate (200 mL) and water (200 mL) and the organic layer was separated. The aqueous phase was extracted with ethyl acetate (3×100 mL) and the combined organic extracts washed with brine (2×80 mL) and dried over MgSO₄. Evaporation afforded the crude di-isopropylidene as the major of two products. The crude mixture was then dissolved in anhydrous methanol (100 mL) under nitrogen at 0° C. before the portionwise addition•of sodiumborohydride (2.9 g 77 mmol). Vigorous effervescence occurred and the solution was stirred at 0° C. for 30 min and then at room temperature for 4 hours. The solvent was removed in vacuo and the residue was partitioned between ethyl acetate (150 mL) and water (10 mL) and the organic layer was separated. The aqueous phase was extracted with ethyl acetate (5×50 mL) and the combined organic extracts washed with brine (2×50 mL) and dried over MgSO₄. Evaporation and chromatography (25% -67% ethyl acetate in petroleum ether) afforded the diol (36) as a colourless, oil (11.44 g, 44 mmol, 82% over 2 steps). R_{f}0.36 (ethyl acetate:hexane) (2:1); [α]_{D}²²-7.9° (c 1.0 in DCM); ¹H NMR (500 MHz, CDCl₃) δ 4.16 (m, 2H), 3.95 (m, 2H); 3.83 (dd, J=11.5, 8.4 Hz, 1H), 3.66 (m, 2H), 3.38 (m, 1H), 1.38 (s, 3H), 1.30 (s, 3H), 1.26 (s, 3H), 1.25 (s, 3H); ¹³C NMR (125 MHz, CDCl₃) δ 109.12, 108.16, 78.25, 76.63, 76.54, 70.19, 67.28, 60.50, 27.51, 27.39, 26.22, 26.01; IR(neat)/cm⁻¹ 3448, 2987, 1737, 1216, 1066; MS (ESI⁺) m/z (rel intensity) 285.25 [100, (M+Na)⁺]; HRMS (ESI⁺) m/z 285.1308 (285.1309 calcd for C₁₂H₂₂O₆Na); Anal. Calcd. for C₁₂H₂₂O₆: C, 54.95; H, 8.45. Found: C, 55.10; H, 8.39. These data agree with the published literature values (Carb. Res. 344 (2009) 1605-1611).

### 2,3,4,6-Di-O-isopropylidene-1,4-anhydro-4-seleno-D-talitol (37)

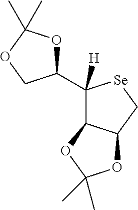 [SMILES: CC1(C)O[C@H]2[C@H]([C@H]3COC(C)(C)O3)[Se]C[C@H]2O1]

To a stirred solution of the diol (36) (5 g, 19 mmol), 4-dimethylaminopyridine (DMAP, 250 mg, 2 mmol) and anhydrous pyridine (10 mL) in dry CH₂Cl₂ (150 mL) under nitrogen at 0° C. was added dropwise methanesulfonyl chloride (4.5 mL, 59 mmol). The solution was stirred at 0° C. for 30 min and then warmed to room temperature for 6 hours. The reaction was quenched by the addition of saturated NaHCO₃ (50 mL) before being extracted with CH₂Cl₂ (3×50 mL). The combined organic extracts were then washed with brine (2×100 mL) and dried over MgSO₄. Evaporation afforded the dimesylate as a yellow oil, which was reacted without further purification. To a stirred suspension of the selenium powder (0.85 g, 10.8 mmol) in degassed ethanol (40 mL) under argon at 0° C. was added a saturated solution of sodiumborohydride (˜1 g) in degassed ethanol (10 mL). The suspension was stirred at 0° C. for 10 min and at room temperature for 1 h during which time the black selenium colour disappeared. The clear solution was then cooled to 0° C. for the addition of the dimesylate (3 g, 7.2 mmol) in THF (5 mL). The reaction mixture was heated and stirred at 70° C. for 12 hours. The solvent was removed in vacuo before the residue was partitioned between ethyl acetate (50 mL) and water (50 mL) and the organic layer was separated. The aqueous phase was extracted with ethyl acetate (3×30 mL) and the combined organic extracts were washed with brine (2×30 mL) and dried over MgSO₄. Evaporation and chromatography (25% ethyl acetate in petroleum ether) afforded the seleno-gulitol (37) as a white crystalline solid (3.18 g, 10.45 mmol, 55% over 2 steps). R_{f}0.49 (hexane:ethyl acetate) (3:1). [α]_{D}²²=−35° (c 1, DCM). ¹H NMR ((CD₃)₂SO) δ 4.95 (ddd, J=5.7, 2.4, 5.5 Hz, 1H), 4.71 (dd, J=2.9, 5.7 Hz, 1H), 4.27 (ddd, 7.5, 6.2 Hz, 1H), 4.08 (dd, J=8.3 Hz, 1H), 3.67 (dd, J=7.4 Hz, 1H), 3.60 (dd, J=5.1 Hz, 1H), 3.22 (dd, 11.3 Hz, 1H), 2.86 (m, 1H), 1.41 (s, 3H), 1.36 (s, 3H), 1.28 (s, 3H), 1.26 (s, 3H). ¹³C NMR ((CD₃)₂SO) δ 110.57, 109.15, 88.42, 85.35, 78.41, 68.95, 51.33, 29.15, 26.72, 26.04, 25.35, 24.30. Anal. calcd. for C₁₂H₂₃O₄Se: C, 46.91; H, 6.56. found: C, 46.81; H, 6.64. These data agree with the published literature values (H. Liu and B. M. Pinto, Can. J Chem., 2006, 84, 4, 497.505).

### 1,4.-Anhydro-4-seleno-D-talitol (38)

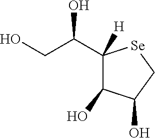 [SMILES: OC[C@@H](O)[C@@H]1[Se]C[C@@H](O)[C@H]1O]

To a stirred solution of the protected seleno sugar (37) (0.5 g, 1.6 mmol) in dry methanol (10 mL) under nitrogen at 0° C. was added acetyl chloride (0.5 mL). The solution was stirred at 0° C. for 10 min and at room temperature for 3 h. The solvent was removed in vacuo and the residue was purified by column chromatography (30% methanol in dichloromethane) afforded the deprotected seleno•sugar (38) as a colourless oil (0.20 g, 0.88 mmol, 55%), recrystallisation from acetone gave a white crystalline solid. R_{f}0.39 (methanol:ethyl acetate) (1:4); ¹H NMR (500 MHz, CDCl₃) δ 4.39 (q, J=3.5 Hz, 1H), 4.03 (ddd, J=7.7, 3.2, 0.8 Hz, 1H), 3.78 (q, J=5.2, Hz, 1H), 3.61 (ddd, J=7.7, 4.7, 0.6 Hz, 1H), 3.50 (ddd, J=5.7, 2.1, 1.1 Hz, 1H), 2.97 (ddd, J=10.5, 4.4, 0.9, J_{H,Se}=12.4 Hz, 1H), 2.70 (ddd, J=10.5, 3.5, 0.7, J_{H,Se}=12.4 Hz, 1H); ¹³C NMR (125 MHz, CDCl₃) δ 79.92, 77.34, 73.29, 67.73, 48.79, 24.78; IR(neat)/cm⁻¹ 3432, 2985, 1217, 1066; MS (ESI⁺) m/z (rel intensity) 251.08 [100, (M+Na)⁺]; HRMS (ESI⁺) m/z 250.9793 (250.9793 calcd for C₆H₁₂O₄SeNa); Anal. Calcd. C₆H₁₂O₄Se: C, 31.74; H, 5.33. Found: C, 31.41; H, 532.

### 1.2 Biological Data

### 1.2.1 Seleno Sugars as Potent Scavengers of Hypochlorous and Hypobromous Acid

The kinetics of the reactions of HOBr (10 μM) with the seleno•sugar derivatives (0.75 mM-0.02 mM) were investigated in competition with N-acetyl tyrosine (1 mM) at 22° C. by the methods described by Davies and co workers (M. J. Davies et al., Antioxid. Redox Signaling, 2008, 10, 7, 1199-1234). The assay examines the conversion of N-acetyl tyrosineto the corresponding N-acetyl-3-bromotyrosine, in the absence and presence of an oxidation scavenger (e.g. Se-sugar). The yields of N-acetyl-3-bromotyrosine at increasing carbohydrate derivative concentration (yield_{quench}) were determined by HPLC and compared to the maximal yield in the absence of added quencher (yieldₘₐₓ). Using competition kinetics, the yields of the products of reaction with HOX are related by equation (1), and rearrangement of this equation results in the linear form (y=mx+c), given in equation (2).

$$\begin{matrix} \frac{{yield}_{quench}}{{yield}_{max} - {yield}_{quench}} = \frac{{k}_{{ }_{Tyr}} [ N - acetyl - Tyr ]}{{k}_{quench} [ quencher ]} & ( 1 ) \\ \frac{{yield}_{max} [ N - acetyl - Tyr ]}{{yield}_{quench}} = \frac{{k}_{quench} [ quencher ]}{{k}_{Tyr}} + [ N - acetyl - Tyr ] & ( 2 )\end{matrix}$$

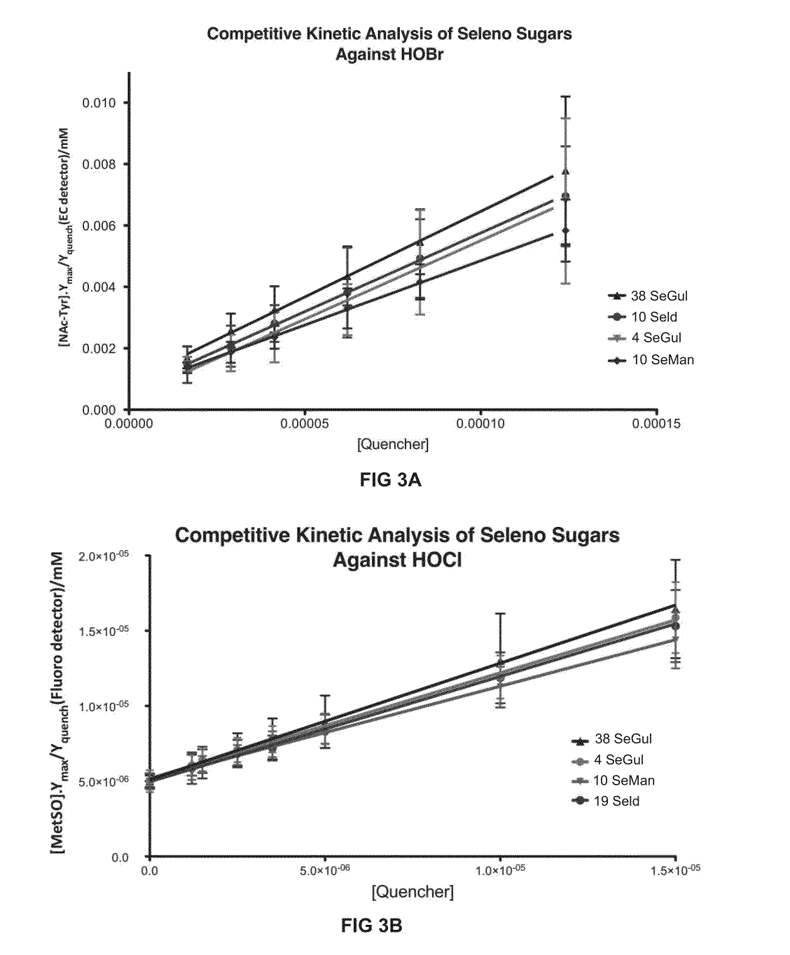

From a plot of yieldₘₐₓ[N-acetyl tyrosine]/yield_{quench} against increasing concentration of quencher ([quencher]) the gradient of the corresponding line can determine the value of k_{quencher} using the known value of k_{Tyr} a set y-intercept equal to [N-acetyl tyrosine]. The results are depicted in FIG. 3A and FIG. 3C.

### Experimental Procedures

### Competitive Kinetic Studies for Seleno-Sugars Against HOBr Using N-Acetyl-Tyrosine

HOBr Preparation.

HOBr was prepared by mixing HOCl (40 mM in water, pH 13) with NaBr (45 mM in water) in equal volumes. The reaction was left for 1 minute before dilution with 0.1 M phosphate buffer (pH 7.4) to the required concentration of HOBr (typically 0.2-2.0 mM). As HOBr disproportionates slowly to form Br⁻ and BrO₂⁻, fresh solutions were prepared for each kinetic run and used within 30 minutes. To investigate whether Br₂ formed in the presence of excess Br⁻ contributed to the observed reaction kinetics, HOBr solutions were also prepared with increasing concentrations of NaBr (45-250 mM). At neutral pH, hypobromous acid exists primarily as HOBr with low concentrations of ⁻OBr also present (pK_{α} 8.7).

### 1.1.1.1 HPLC Instrumentation and Methods

Analysis and quantification of N-acetyl-tyrosine and its reaction products with HOBr were carried out on a Shimadzu LC-10A HPLC system (Shimadzu, South Rydalmere, NSW, Australia). The reaction mixtures were separated on a Zorbax reverse-phase HPLC column (25 cm×4.6 mm, 5 μM particle size; Rockland Technologies, Newport, Del.) packed with octadecyl silanized silica, equipped with a Pelliguard guard column (2 cm; Supelco). The column was maintained at 30° C. using a column oven (Waters Corp., Milford, Mass.). The mobile phase was comprised of a gradient of solvent A (10 mM phosphoric acid with 100 mM sodium perchlorate at pH 2.0) and solvent B [80% (v/v) MeOH in nanopure water] eluting at 1 mL min⁻¹. The gradient was programmed as follows: 20% solvent B and 80% solvent A at 0 min increasing to 80% solvent Boyer 10 mins; over the next 5 minutes the proportion of solvent B was held at 80%, before the proportion of solvent B was reduced to 20%, and the column was allowed to re-equilibrate for 6 minutes prior to injection of the next sample. The eluent was monitored in series by a UV detector (280 nm) and an electrochemical detector (Antec Leyden Intro). The channel of the electrochemical detector was set to an oxidation potential of +1200 mV to quantify the halogenated N-acetyl-tyrosine products. Peak areas were quantified using Class VP 7.4 Sp1 software (Shimadzu) and compared to authentic standards when required. Using these conditions, N-acetyl-tyrosine was detected in the +1200 mV electrochemical channel at a retention time of 8.3 min, N-acetyl-3-bromotyrosine at 11.4 min, and N-acetyl-3,5-dibromotyrosine at 13.4 min. A small impurity peak present in the parent compound was also detected with a retention time of 5.6 min, but this was not characterized further.

### 1.1.1.2 Sample Preparation for HOBr Analysis

Varying concentrations of Se-sugars (0.75 mM-0.02 mM) were added to solutions of known N-acetyl-tyrosine (1 mM).

### 1. Sample Compositions for HOBr (10 μM) Rate Determination for Se-Sugars

| | Final | Final | | Vol | |
|---|---|---|---|---|---|
| Sample | [Se-sugar] | [NAcTyr] | | NAcTyr | Vol |
| no. | mM | mM | Vol Se-Gul | (2 mM) | PB |
| Se1 | 0.750 | 1 | 15 μL of 10 mM | 110 μL | 95 μL |
| Se2 | 0.500 | 1 | 100 μL of 1 mM | 110 μL | 10 μL |
| Se3 | 0.350 | 1 | 70 μL of 1 mM | 110 μL | 40 μL |
| Se4 | 0.200 | 1 | 40 μL of 1 mM | 110 μL | 70 μL |
| Se5 | 0.150 | 1 | 30 μL of 1 mM | 110 μL | 80 μL |
| Se6 | 0.100 | 1 | 20 μL of 1 mM | 110 μL | 90 μL |
| Se7 | 0.075 | 1 | 15 μL of 1 mM | 110 μL | 95 μL |
| Se8 | 0.050 | 1 | 10 μL of 1 mM | 110 μL | 100 μL |
| Se9 | 0.035 | 1 | 7 μL of 1 mM | 110 μL | 103 μL |
| Se10 | 0.020 | 1 | 4 μL of 1 mM | 110 μL | 106 μL |
| Control | 0.000 | 1 | 0 | 110 μL | 110 μL |

200 μL of each sample (Se1-Se10) was subsequently added to a solution of HOBr (20 μL of 0.1 mM HOBr). Samples, with a final volume of 200 μL, were then mixed, filtered (0.2 μM cut-off filters) arid placed in a glass HPLC vials for HPLC analysis.

### Competitive Kinetic Studies for Seleno-Sugars Against HOCl Using FMoc-Methionine

The kinetics of the reactions of HOCl (1 μM) with the seleno-sugar derivatives (1.2 μM-20 μM) were investigated in competition with FMoc-methionine (5 μM) at 22° C. by adapting the methods described by Davies and co-workers (M. J. Davies et al., Antioxid. Redox Signaling, 2008, 10, 7, 1199-1234). The assay examines the conversion of FMoc-methionine to the corresponding FMoc-methionine sulfoxide, in the absence and presence of an oxidation scavenger (e.g. Se-sugar). The yields of FMoc-methionine sulfoxide at increasing carbohydrate derivative concentration (yield_{quench}) were determined by HPLC and compared to the maximal yield in the absence of added quencher (yieldₘₐₓ). Using competition kinetics, the yields of the products of reaction with HOX are related by equation (1), and rearrangement of this equation results in the linear form (y=mx+c), given in equation (2).

$$\begin{matrix} \frac{{yield}_{quench}}{{yield}_{max} - {yield}_{quench}} = \frac{{k}_{{ }_{Tyr}} [ N - acetyl - Tyr ]}{{k}_{quench} [ quencher ]} & ( 1 ) \\ \frac{{yield}_{max} [ N - acetyl - Tyr ]}{{yield}_{quench}} = \frac{{k}_{quench} [ quencher ]}{{k}_{Tyr}} + [ N - acetyl - Tyr ] & ( 2 )\end{matrix}$$

From a plot of yieldₘₐₓ[FMoc-methionine]/yield_{quench} against increasing concentration of quencher ([quencher] the gradient of the corresponding line can determine the value of k_{quencher} using the known value of k_{Met} with a set y-intercept equal to [FMoc-methionine]. The results are depicted in FIG. 3B and FIG. 3C.

### Experimental Procedures

All chemicals were obtained from Sigma/Aldrich/Fluka and were used as received, with the exception of sodium hypochlorite (in 0.1 M NaOH, low in bromine; BDH Chemicals). The HOCl was standardized by measuring the absorbance at 292 nm at pH 12 [ε-292(⁻OCl)350 M⁻¹ cm⁻¹]. All studies were performed in 10 mM phosphate buffer (pH 7.4). All phosphate buffers were prepared using Milli Q water and treated with Chelex resin (Bio-Rad) to remove contaminating transition metal ions. The pH values of solutions were adjusted, where necessary, to pH 7.4 using 100 mM H₂SO₄ or 100 mM NaOH.

### 1.1.1.3 HPLC Instrumentation and Methods

Analysis and quantification of FMoc-methionine and its reaction products with HOCl were carried out on a Shimadzu Nexera UPLC system (Shimadzu, South Rydalmere, NSW, Australia). The reaction mixtures were separated on a Shim-pack XR-ODS (Shimadzu, 100×4.6 mm, 2.2 μM) column. The column was maintained at 40° C. with a flow rate of 1.2 mL·min⁻¹. The mobile phase was comprised of a gradient of solvent A[(MeOH (20%), THF (2.5%), NaOAc (5%) and H₂O (72.5%)] and solvent B [MeOH (80%), THF (2.5%) and NaOAc (5%), H₂O (12.5%)]. The gradient was programmed as follows: 75% solvent B and 25% solvent A at 0 min, increasing to 87.5% solvent B over 5 min, followed by a further increase to 100% solvent B over the next 0.5 min and a wash with 100% solvent B for 2.5 min, before returning to 75% solvent B over the next 0.5 min with 3.5 min of re-equilibrating preceding the next injection. The eluent was monitored by fluorescence detection (RF-20Axs; λₑₓ, 265 nm; λₑₘ, 310 nm), with peak areas determined using Lab solutions 5.32 SP1 software (Shimadzu) and compared to authentic standards when required. Using these conditions, FMoc-methionine sulfoxide was detected in the fluorescence channel (λₑₓ, 265 nm; λₑₘ, 310 nm) at a retention time of 1.7 min, and FMoc-methionine at 2.8 min.

### 1.1.1.4 Sample Preparation for HOCl Analysis

Varying concentrations of Se-sugars (1.2 μM-20 μM) were added to solutions of known FMoc-methionine (5 μM).

### 2. Sample Compositions for HOCl (1 μM) Rate Determination for Se-Sugars

| | Final | Final | | Vol | |
|---|---|---|---|---|---|
| Sample | [Se-sugar] | (FMocMet] | | FMocMet | Vol |
| no. | μM | μM | Vol Se-Gul | (50 μM) | PB |
| Se0 | 0 | 5 | 0 | 50 μL | 200 μL |
| Se1 | 1.2 | 5 | 6 μL of 0.1 mM | 50 μL | 194 μL |
| Se2 | 1.5 | 5 | 7.5 μL of 0.1 mM | 50 μL | 192.5 μL |
| Se3 | 2.5 | 5 | 12.5 μL of 0.1 mM | 50 μL | 187.5 μL |
| Se4 | 3.5 | 5 | 17.5 μL of 0.1 mM | 50 μL | 182.5 μL |
| Se5 | 5 | 5 | 25 μL of 0.1 mM | 50 μL | 175 μL |
| Se6 | 10 | 5 | 50 μL of 0.1 mM | 50 μL | 150 μL |
| Se7 | 15 | 5 | 75 μL of 0.1 mM | 50 μL | 125 μL |
| Se8 | 20 | 5 | 100 μL of 0.1 mM | 50 μL | 100 μL |
| Blank | 0 | 5 | 0 | 50 μL | 450 μL |

250 μL of 2 μM HOCl was added to each sample (Se0-Se8) except the Blank. Samples, with a final volume of 500 μL, were then mixed, filtered (0.2 μM cut-off filters) and placed in a glass HPLC vials for HPLC analysis.

### HPLC Amino Acid Analysis of HOCl Oxidized BSA and Plasma

### 1.1.1.5 Sample Preparation for Protein Hydrolysis

Varying concentrations of Se-sugars (1.0 mM-0.05 mM) were added to solutions containing 0.1 mg·mL⁻¹ of protein (BSA or Plasma).

### 3. Example of Sample Compositions for Protein Protection Against HOCl (0.76 mM) by Se-Sugars

| Sample | [Se-Sugar] | Protein | Vol | Protein Vol | Vol | Total |
|---|---|---|---|---|---|---|
| no. | mM | mg · mL⁻¹ | Se-Gul | [1 mg · mL⁻¹] | buffer | Vol |
| Se1BSA1 | 1.000 | 0.5 | 21.3 μL of | 106.7 μL | 32 μL | 160 μL |
| | | | 10 mM | | | |
| Se2BSA1 | 0.500 | 0.5 | 10.7 μL of | 106.7 μL | 42.6 μL | 160 μL |
| | | | 10 mM | | | |
| Se3BSA1 | 0.200 | 0.5 | 42.6 μL of | 106.7 μL | 10.7 μL | 160 μL |
| | | | 1 mM | | | |
| Se4BSA1 | 0.100 | 0.5 | 21.3 μL of | 106.7 μL | 32 μL | 160 μL |
| | | | 1 mM | | | |
| Se5BSA1 | 0.050 | 0.5 | 10.7 μL of | 106.7 μL | 42.6 μL | 160 μL |
| | | | 1 mM | | | |
| SeConBSA | 0.000 | 0.5 | 0 | 106.7 μL | 53.3 μL | 160 μL |
| Control | 0.000 | 0.5 | 0 | 100 μL | 100 μL | 200 μL |

150 μL of each sample (Se1BSA1—Control) were added to 50 μL of 3 mM HOCl. Samples, with a final volume of 200 μL, were placed in a glass vial (8×40 mm, 1 mL, No. 98212, Alltech) labeled by etching with a diamond tipped pen or engraver. Proteins (0.1 mg in 200 μL) were delipidated and precipitated by the addition of 25 μL 0.3% (w/v) deoxycholic acid and 50 μL of 50% (w/v) TCA, with incubation on ice for 5 min. The glass vials containing samples were placed in 1.5 mL centrifuge tubes (with caps removed) for 2 minutes at 9000 rpm at 5° C. (Eppendorf 5415R centrifuge) to pellet protein. Protein pellets were washed once with 5% (w/v) TCA, and twice with ice cold acetone (stored in −20° C. freezer) with 2 min, 9000 rpm, spins between washes in each case to settle pellets. Samples were then re-suspended in 150 μL of 4 M methanesulfonic acid (MSA) containing 0.2% w/v tryptamine, before the addition of 5 μL of homo-Arg (10 mM) as an internal standard. The samples were then transferred to PicoTag hydrolysis vessels and placed under vacuum in the oven at 110° C. for 16-18 hours. The PicoTag vessels were removed from oven and allowed to cool before releasing vacuum. Samples were neutralized by the addition of 150 μL freshly prepared 4 M NaOH and filtered (centrifuge at 10,000 rpm for 2 minutes through a PVDF 0.22 μm membrane, 0.5 mL volume, No. UFC30GVNB, Millipore) to remove any insoluble precipitate. The samples were diluted into water (10-fold), before transferring 40 μL to HPLC vials.

### 1.1.1.6 Preparation of OPA and Amino Acid Standards

OPA reagent (Sigma-Aldrich, P7914) was activated immediately before use by addition of 5 μL of 2-mercaptoethanol to 1 mL of OPA reagent in a HPLC vial. The derivatization method involved 20 μL injections of activated OPA reagent per sample. A solution of 5 μM standards was prepared by addition of 10 μL Sigma-Aldrich amino acid standards (A9781, 500 μL stock), 5 μL MetSO (1 mM stock), and 5 μL homo-Arg (1 mM stock) to 980 μL water. These stock solutions were diluted to give 1, 2, 3, 4, and 5 μL standards. 40 μL of each standard was transferred to HPLC vials containing 0.2 mL inserts and placed in the auto injector.

### 1.1.1.7 Preparation of HPLC Mobile Phase

A 1.0 M stock solution of sodium acetate trihydrate was prepared by the. addition of 136.08 g of this compound to•900 mL of water, before pH adjustment to 5.0 with glacial acetic acid (˜29 mL) before addition of water to a final volume of 1 L. Buffer A contained 400 mL MeOH, 50 mL tetrahydrofuran; 1450 mL water, and 100 mL of 1.0 M sodium acetate, pH 5.0 (to give 50 mM final). Buffer B contains 1600 mL MeOH, 50 mL tetrahydrofuran, 250 mL water, and 100 mL of 1 M sodium acetate, pH 5.0 (to give 50 mM final). Both buffers were filtered through 0.2 μm membrane filters (e.g., VacuCap 90 filter unit with 0.2 μm Supor membrane, No. 4622, Pall Corporation), and degassed prior to running HPLC analysis.

### 1.1.1.8 HPLC Conditions, Method and Results

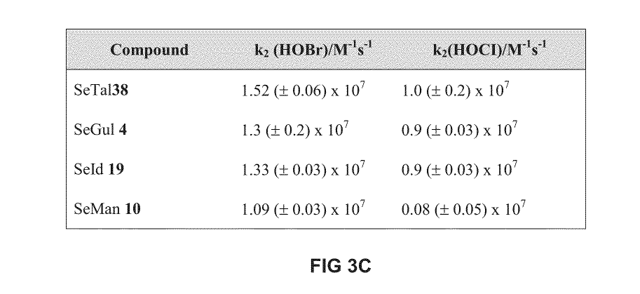

The auto injector was programmed to add 20 μL activated OPA reagent to the specified sample (40 μL), followed by 3 mixing cycles, and a 1 minute incubation period. After the incubation step, 15 μL of the final reaction mixture was injected. A flow rate of 1 mL min⁻¹ was used, with the column oven set at 30° C. and fluorescence detector set with λ_{EX} 340 nm, λ_{EM} 440 nm. The concentration of each amino acid in the samples was determined from linear plots of the HPLC peak area versus concentration from the standards. Any variation in derivatization efficiency was taken into account by expressing the results as a ratio with the internal standard homo-Arg. Any variation in the efficiency of hydrolysis or sample recovery after the precipitation and washing steps was taken into account by expressing the concentration of the amino acids of interest as a ratio with an amino acid that is not modified by the particular oxidant treatment. The results showing protection of individual amino acid residues present on BSA are depicted in FIGS. 4A-E, and analogous data for the protection of amino acid residues present on proteins in human plasma are shown in FIGS. 4F-J.

### Analysis of 3-Chlorotyrosine Using LCMS

### 1.1.1.9 Sample Preparation for Protein Hydrolysis

Varying concentrations of Se-sugars (1.0 mM-0.05 mM) were added to solutions containing 0.1 mg·mL⁻¹ of protein (BSA or Plasma).

### 4. Example of Sample Compositions for Cl-Ty Prevention Against HOCl (0.76 mM) by Se-Sugars

| Sample | [Se-Sugar] | Protein | Vol | Protein Vol | Vol | Total |
|---|---|---|---|---|---|---|
| no. | mM | mg · mL⁻¹ | Se-Gul | [1 mg · mL⁻¹] | buffer | Vol |
| Se1HSA1 | 1.000 | 0.5 | 21.3 μL of | 106.7 μL | 32 μL | 160 μL |
| | | | 10 mM | | | |
| Se2HSA1 | 0.500 | 0.5 | 10.7 μLof | 106.7 μL | 42.6 μL | 160 μL |
| | | | 10 mM | | | |
| Se3HSA1 | 0.200 | 0.5 | 42.6 μL of | 106.7 μL | 10.7 μL | 160 μL |
| | | | 1 mM | | | |
| Se4HSA1 | 0.100 | 0.5 | 21.3 μL of | 106.7 μL | 32 μL | 160 μL |
| | | | 1 mM | | | |
| Se5HSA1 | 0.050 | 0.5 | 10.7 μL of | 106.7 μL | 42.6 μL | 160 μL |
| | | | 1 mM | | | |
| SeConHSA | 0.000 | 0.5 | 0 | 106.7 μL | 53.3 μL | 160 μL |
| Control | 0.000 | 0.5 | 0 | 100 μL | 100 μL | 200 μL |

150 μL of each sample (Se1HSA1—Control) were added to 50 μL of 3 mM HOCl. Samples, with a final volume of 200 μL, were placed in a glass vial (8×40 mm, 1 mL, No. 98212, Alltech) labeled by etching with a diamond tipped pen or engraver. Proteins (0.1 mg in 200 μL) were delipidated and precipitated by the addition of 25 μL 0.3% (w/v) deoxycholic acid and 50 μL of 50% (w/v) TCA, with incubation on ice for 5 min. The glass vials containing samples were placed in 1.5 mL centrifuge tubes for 2 minutes at 9000 rpm at 5° C. (Eppendorf 5415R centrifuge) to pellet protein. Protein pellets were washed once with 5% (w/v) TCA, and twice with ice cold acetone (stored in 20° C. freezer) with 2 min, 9000 rpm, spins between washes in each case to settle pellets. The samples were then transferred to PicoTag hydrolysis vessels before the addition of 150 μL of 6 M HCl and 50 μL of thioglycolic acid into the PicoTag vessel and placed under vacuum in the oven at 110° C. for 16-18 hours. The PicoTag vessels were removed from oven and allowed to cool before releasing vacuum. The sample vials were then placed in 1.5 mL centrifuge tubes and dried under vacuum, using centrifuge speedy vacuum system (3 hours at maximum vacuum). Each sample was then re-suspended in 50 μL of water and filtered (centrifuge at 10,000 rpm for 2 minutes through a PVDF 0.22 μL membrane, 0.5 mL volume, No. UFC30GVNB, Millipore) to remove any insoluble precipitate. The samples were then transferred to HPLC vials for LCMS analysis.

### 1.1.1.10 Preparation of Standards

A standard solution of 100 μM tyrosine and 2.5 mM 3-chlorotyrosine was prepared in buffer. Each stock was diluted to give 1:1 mixtures of tyrosine:chlorotyrosine with concentrations of 100-500 pmol in 20 μM. 40 μL of each standard was transferred to HPLC vials for LCMS analysis.

### 1.1.1.11 Sample Analysis

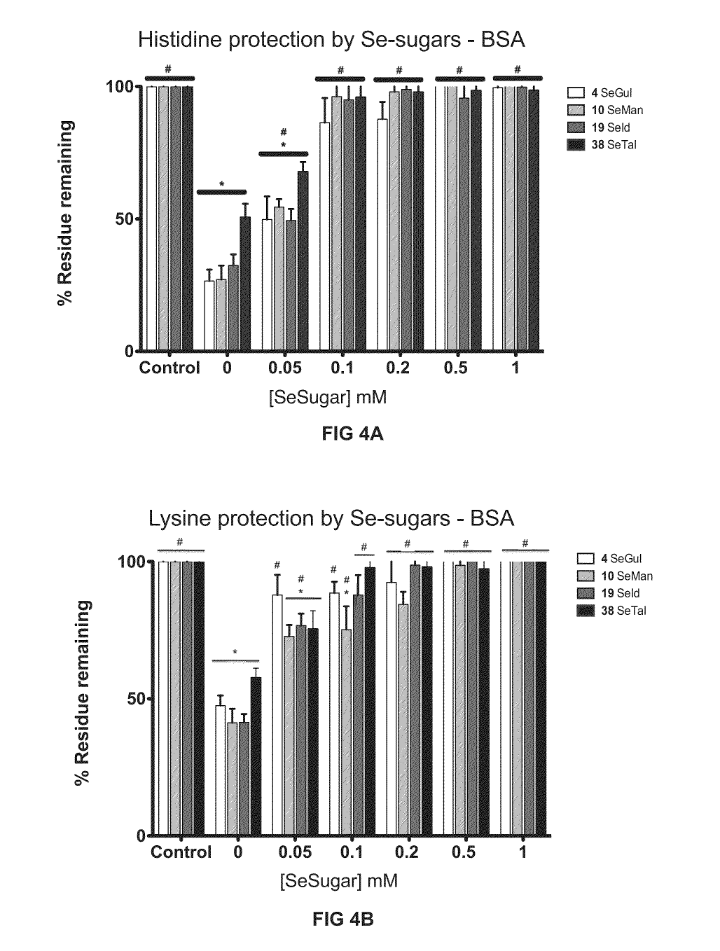

L-Tyrosine, 3-chlorotyrosine and di-tyrosine were analysed by LC-MS in the positive ion mode with a Finigan LCQ Deca XP ion-trap instrument coupled to a Finigan surveyor HPLC system. Tyrosine residues were separated on a Thermo hypercarb ODS column (100 mm×2.1 mm; 5 μm particle size) at 30° C. with a flow rate of 0.2 mL·min⁻¹. Solvent A contained 0.1% TFA in water and solvent B contained 0.1% TFA in acetonitrile. The tyrosine residues were eluted using the following gradient: 5% to 50% B over 20 minutes, then 50-80% B over 2 minutes, followed by isocratic elution of 80% B for 5 minutes before decreasing to 5% B for 3 minutes and re-equilibrating to 5% B for 20 minutes. The electrospray needle was held at 4500 V. Helium was used as the collision gas and nitrogen was used as the sheath and sweep gas set to 50 and 32 units respectively. The temperature of the heated capillary was 325° C. The results are shown in FIGS. 5A and 5B.

### Scavenging of HOCl and Chloramines Using TMB Assay

### 1.1.1.12 Basis of TMB Assay

The developing reagent was prepared by dissolving 4.8 mg of TMB in 1 mL of dimethylformamide, followed by the addition of 9 mL of 0.44 M pH 5.4 sodium acetate buffer and 50 μL of 2 mM sodium iodide solution. The developing reagent was prepared immediately prior to addition to the standards and samples to avoid any unwanted oxidation of TMB. Standard curves were produced by adding varying amounts, between 0 and 100 μL, 200 μM HOCl, to 100 μL of 10 mM taurine solution in a 96-well plate. The volume in each well was made up to 200 μL with 0.1 M pH 7.4 phosphate buffer. The standards were incubated for 5 minutes before the addition of developing reagent. The solution was incubated for another 5 minutes before the absorbance at 645 nm was determined using BioRad Benchmark Plus microplate spectrophotometer. Standards were produced substituting 10 mM taurine solution with 10 mM solutions of glycine and N-acetyl-lysine, 200 μM solution of N-acetyl-histidine and 0.5 mg/mL solution of bovine serum albumin or human plasma.

Chloramines were formed by adding. 50 μL of 200 μM HOCl solution to 10 mM taurine solution and incubated for 5 minutes. Varying volumes, between 0 and 50 μL, of 400 μM potential antioxidant solution were then added to the wells, and the volume made up to 200 μL in each well with 0.1 M pH 7.4 phosphate buffer. The samples were incubated for 5 minutes before the addition of developing reagent. The solution was incubated for another 5 minutes before the absorbance at 645 nm was determined using BioRad Benchmark Plus microplate spectrophotometer. The method was repeated substituting the 10 mM taurine solution with 10 mM solutions of glycine and N-acetyl-lysine, 200 μM N-acetyl-histidine and 0.5 mg/mL bovine serum albumin or human plasma.

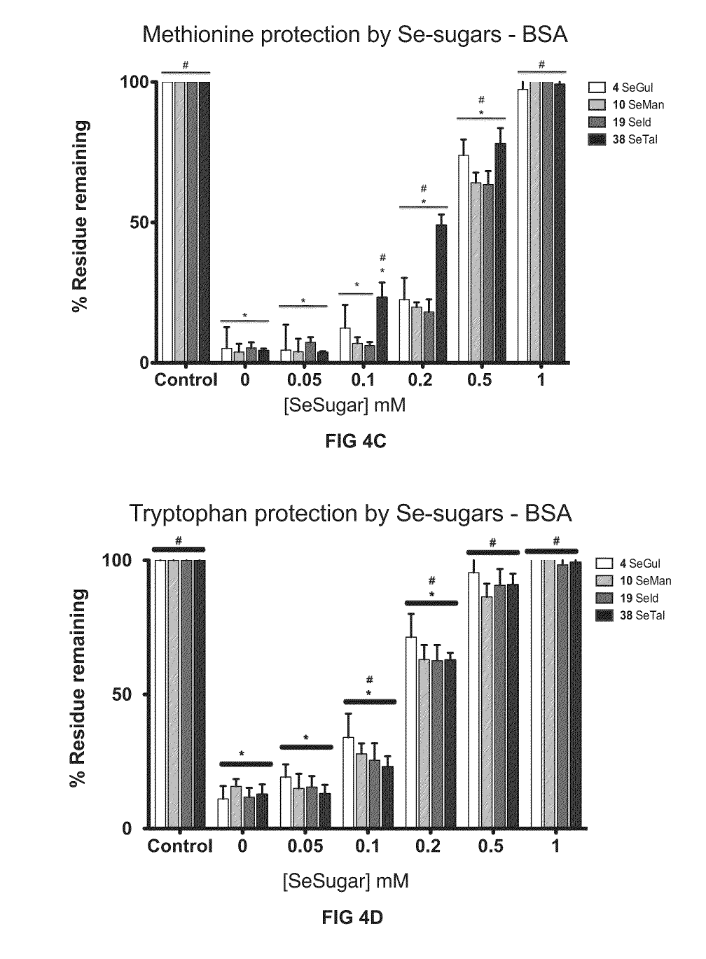

The results are shown in FIGS. 6A to 6E. The IC50 values for scavenging of the various chloramines by the compounds tested are given in FIG. 6F.

### Recycling of Oxidized Seleno Compounds by Thiols

The purpose of these experiments was to determine whether thiols could reduce the selenoxides formed on oxidation of the seleno compounds. The ThioGlo assay was used to monitor the loss of thiol groups upon addition of selenoxides as this agent produces a fluorescent product in the presence of reduced thiols.

### ThioGio Assay Method

The ThioGlo reagent was prepared by diluting 30 μL of a stock solution (5 mg in 5.070 mL acetonitrile) in 2970 μL of 0.1 M pH 7.4 phosphate buffer. Preparation of the developing reagent was performed immediately prior to addition to standards or samples. Standard curves were prepared by addition of varying volumes, between 0 and 50 μL, of 10 μM GSH solution to wells in a 96-well plate. The volume in each well was made up to 50 μL with 0.1 M pH 7.4 phosphate buffer. 50 μL of ThioGlo•reagent was added to each standard, and incubated in the dark for 5 minutes. The fluorescence was measured using a PerSeptive Biosystems CytoFluor II fluorescence multi-well plate reader with λₑₓ=360 nm λₑₘ=530 nm. Standards using 10 μM Cys and 2 mg/mL BSA were produced in the same method.

Solutions of 8 μM SeMetO were produced by mixing 20 μM SeMet and 16 μM HOCl together, and incubating for 30 minutes. Samples were prepared by adding 25 μL of 16 μM to wells of a 96-well plate. Varying volumes of 8 μM SeMetO, between 0 and 25 μL, were added to the samples, and the volume of each made up to 50 μL using 0.1 M pH 7.4 phosphate buffer. 50 μL of ThioGlo reagent was added to each sample, and incubated in the dark for 5 minutes. The fluorescence was measured using the PerSeptive Biosystems CytoFluor II fluorescence multi-well plate reader with λₑₓ=360 nm λₑₘ=530 nm. Samples using 16 μM cysteine and 3 mg/mL bovine serum albumin in place of 16 μM glutathione, were produced in the same method. Samples using 8 μM SeTalO, in place of 8 μM SeMetO, were produced in the same method. Standard curves for GSH, BSA and Cys had R² values >0.99.

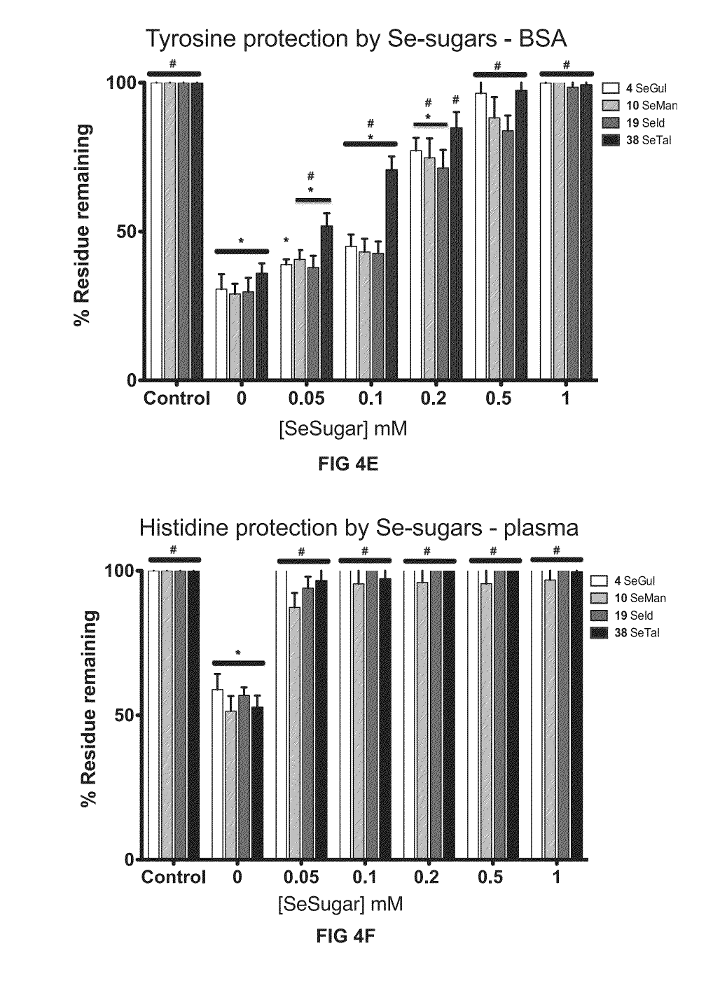

The results are reported as a percentage of thiol remaining after selenoxide addition. GSH and Cys samples showed a dose dependent decreases in the amount•of thiols after addition of the selenoxides from selenomethionine (SeMetO) and 1,4-anhydro-4-seleno-D-talitol (SeTalO) consistent with a dose dependent reduction of the pre-formed selenoxide back to the parent selenide. This reduction was less marked with the thiol group present on bovine serum albumin (FIGS. 7A and B).

### Cytotoxic Effects of Seleco-Compounds

C57Bl/6 mouse isolated glial cells and Chinese Hamster Ovary (CHO) were kindly donated by Dr Peter Crack (University of Melbourne) and Prof. Walter Thomas (University of Queensland, Australia), respectively. Cells were cultured in a tissue-culture flask containing Modified Eagles Medium (MEM) and 50% Foetal Bovine Serum (FBS). The cells were grown in a 5% CO₂ incubator (Forma Scientific, Marietta, Ohio, USA) at 37° C. until they were confluent. Once confluent, cells were plated onto a 96 well plate at a density of 30,000 cells per well.

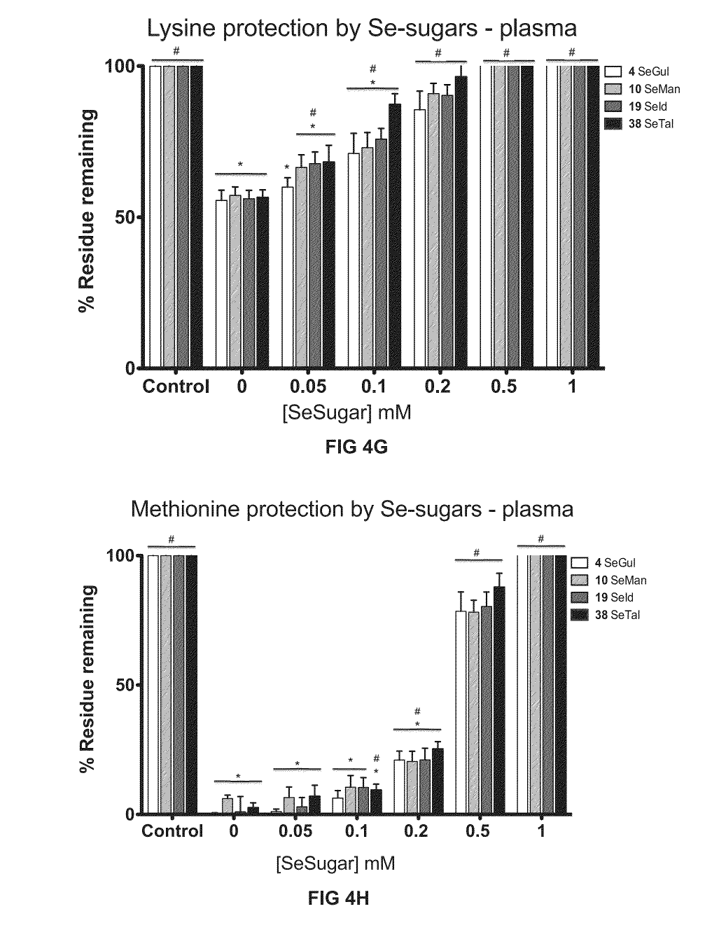

Wells were incubated with phosphate buffered saline (PBS), SeTal (compound 38) (1 mm), SeGul (compound 4) (1 mM) or staurosporine (0.01, 0.1 or 1 μM) in quadruplicates for 48 h in a 5% CO₂ incubator. Drugs were made up fresh daily in PBS. After 48 h cells were incubated for 2 h with 3-(4,5-dimethylthiazol-2-yl)-2,5-diphenyltetrazolium bromide (MTT; 2 mg/ml). After 2 h the media was decanted and cells were then solubilised with 25% dimethyl sulfoxide (DMSO). The contents of each well was then transferred to a Clean 96 well plate and the absorbance of the wells determined, using spectrophotometery (Thermo Electron Corporation, Vantaa, Finland) at 595 nm λ. (FIG. 8).

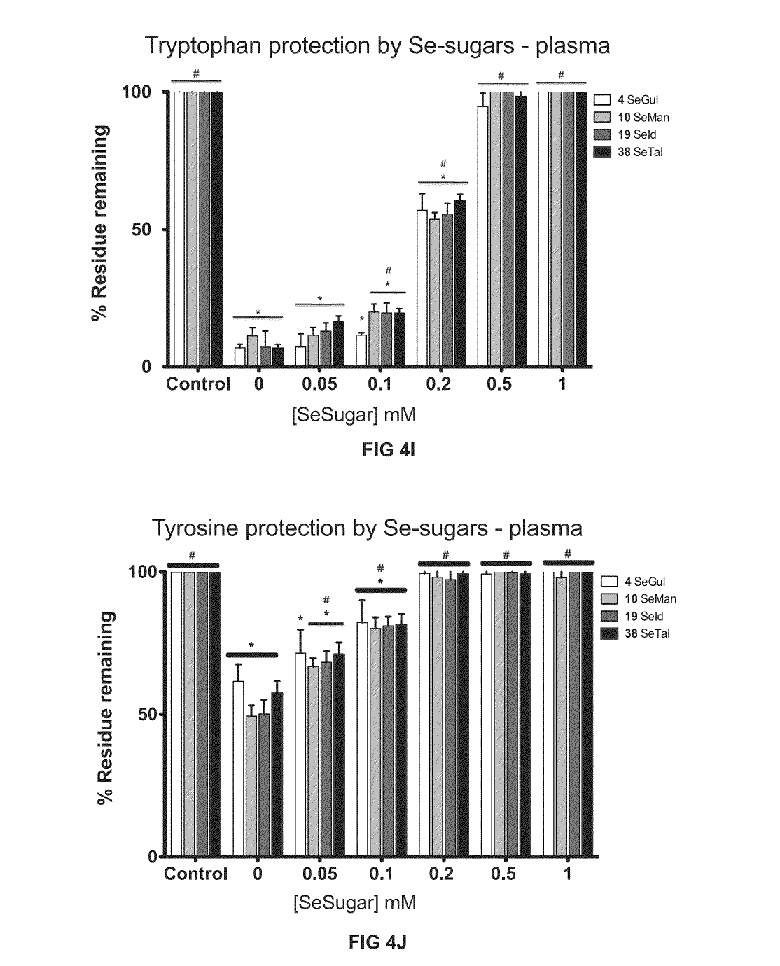

MTT is a yellow tetrazole which is converted by the mitochondrial reductase of living cells into a purple formazan. DMSO is added to each well to dissolve the insoluble purple formazan product into a coloured solution. Absorbance of the wells was averaged for each treatment group and expressed as a percentage of control wells (% control) which were incubated with PBS only. Differences in cell survival were compared using a one-sample test compared to control (100%; GraphPad, La Jolla, Calif., USA). The results are depicted in FIG. 9.

### Effects of Seleno-Compounds on Diabetic Wounds

The seleno-compounds of the present invention have been experimentally tested by topical application to wounds exhibited in a normal (wildtype) mouse model, a diabetic (db/db) mouse model, and compared with a non-treated control.

Experimental data (discussed in detail below) indicate that diabetic mice showed significantly slower wound closure compared to wildtype mice in the absence of treatment using seleno-compounds according to the present invention. With administration of seleno-compounds, significant improvements in tissue repair were detected, with wound closure 3-fold better than control db/db mice at day 4 (32% vs 11%, p<0.001) and 2 fold better at day 10 (83% vs 45%, p<0.0001).

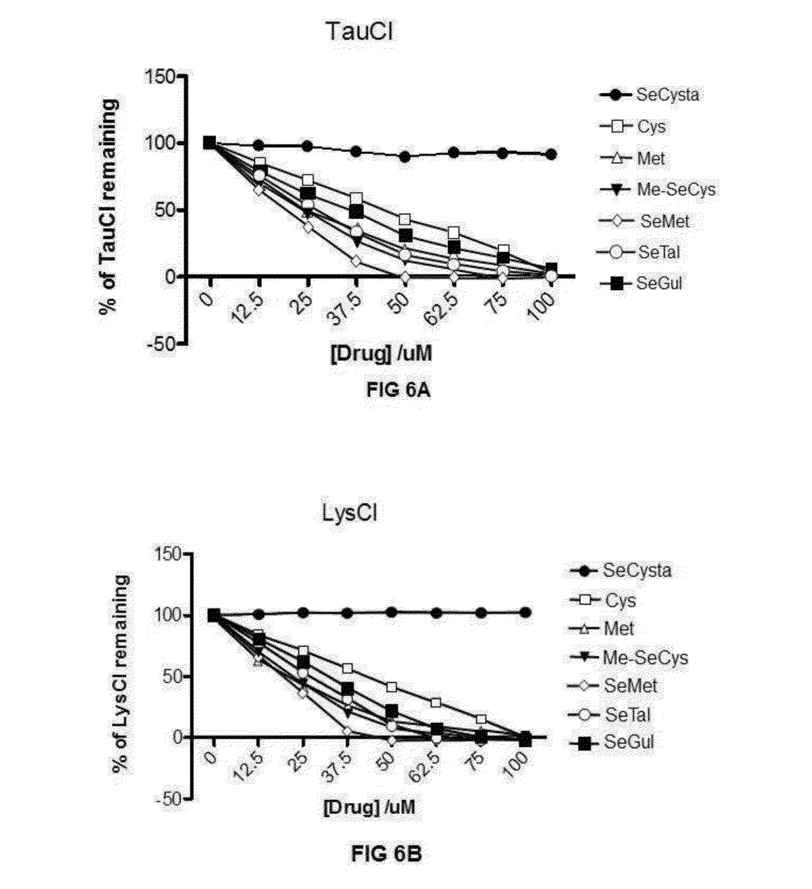

Doppler imaging (FIG. 11) showed improved vascular perfusion (p<0.05) in diabetic but not wildtype mouse, wounds treated with seleno-compounds of the present invention.

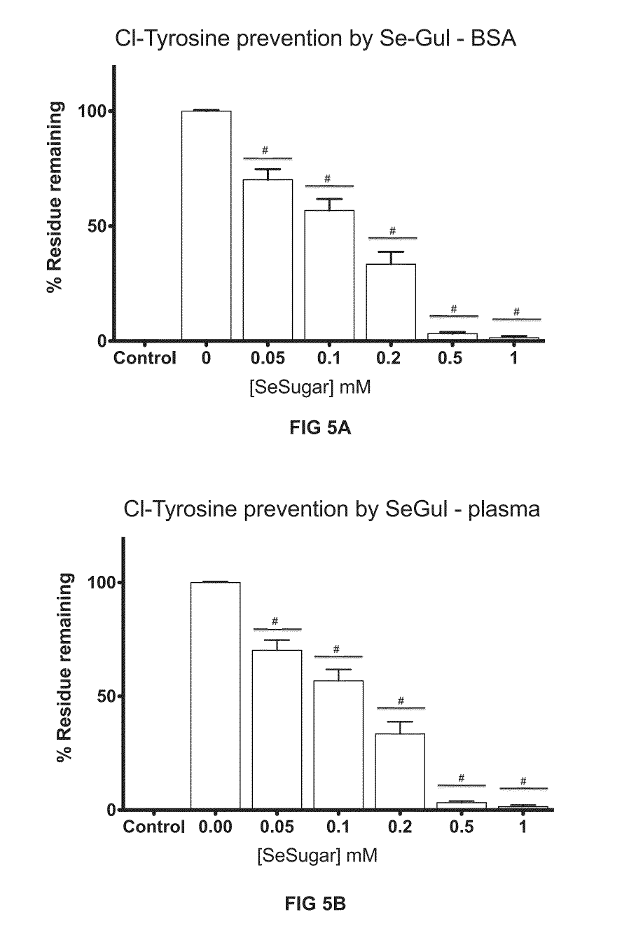

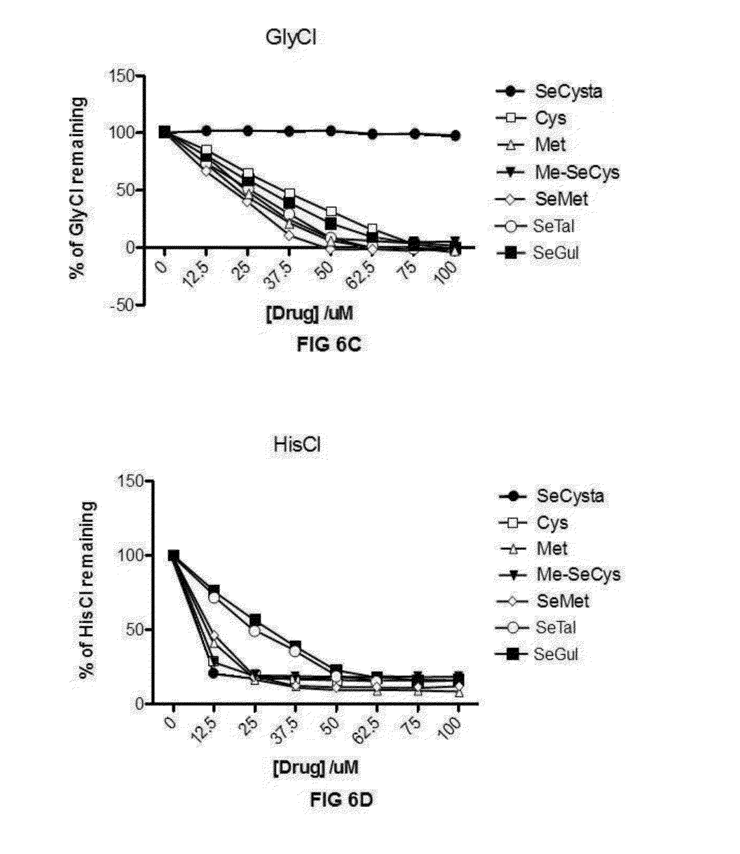

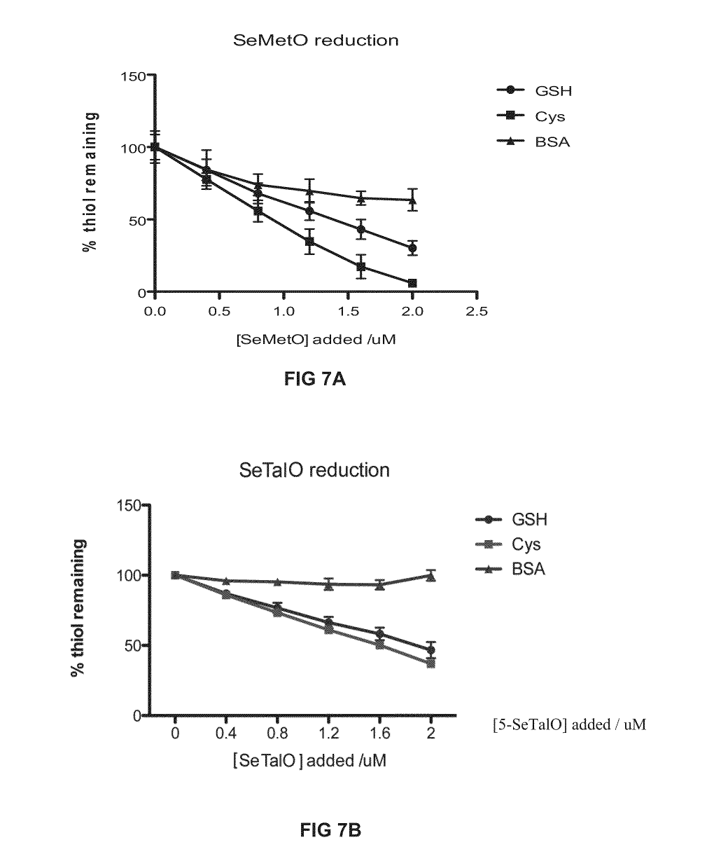

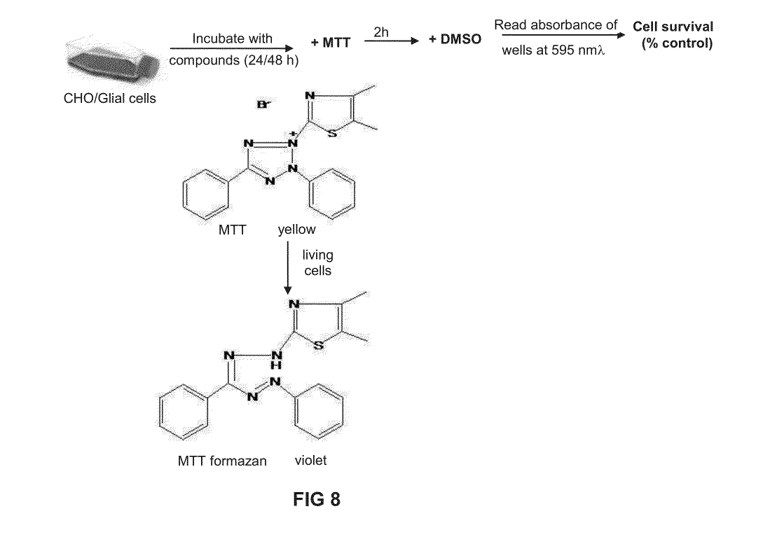

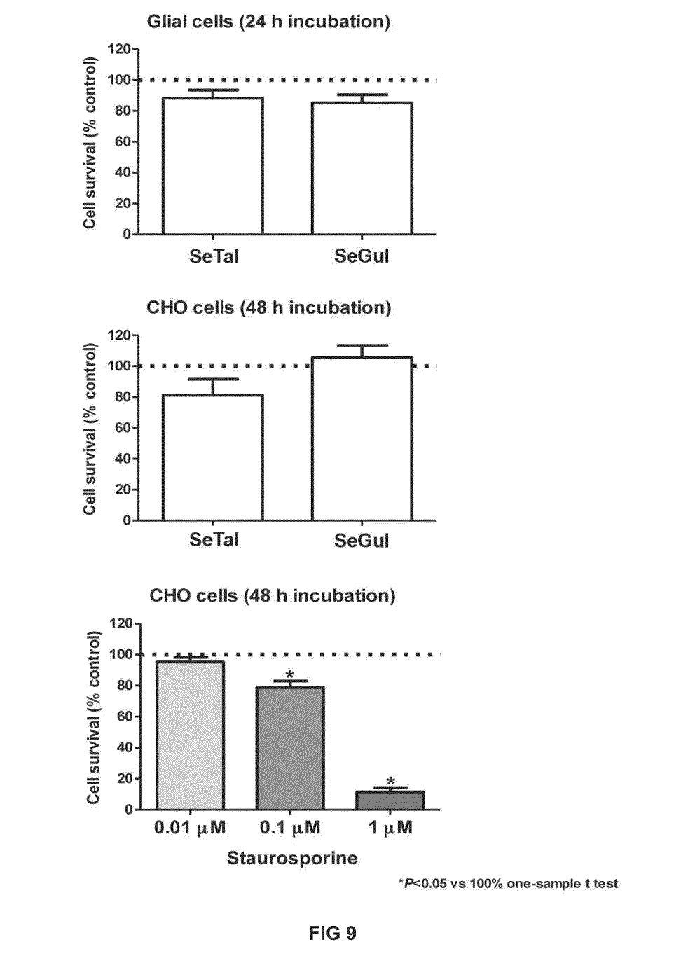

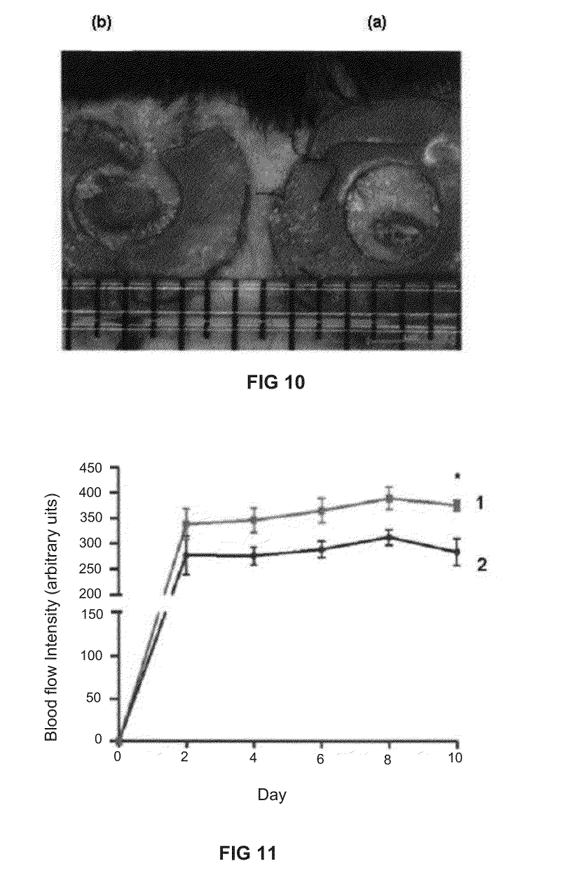

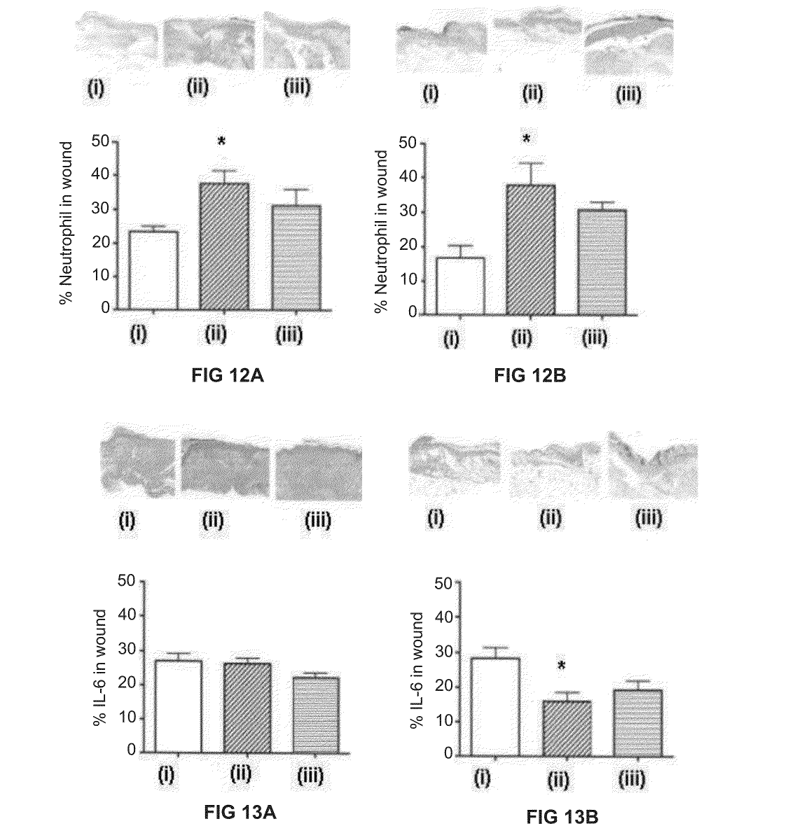

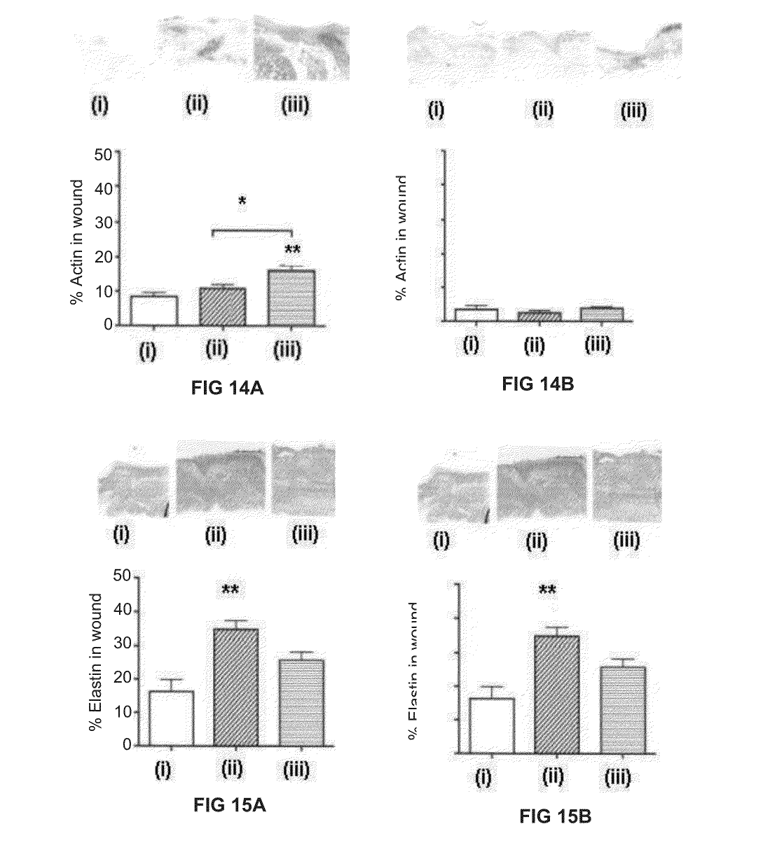

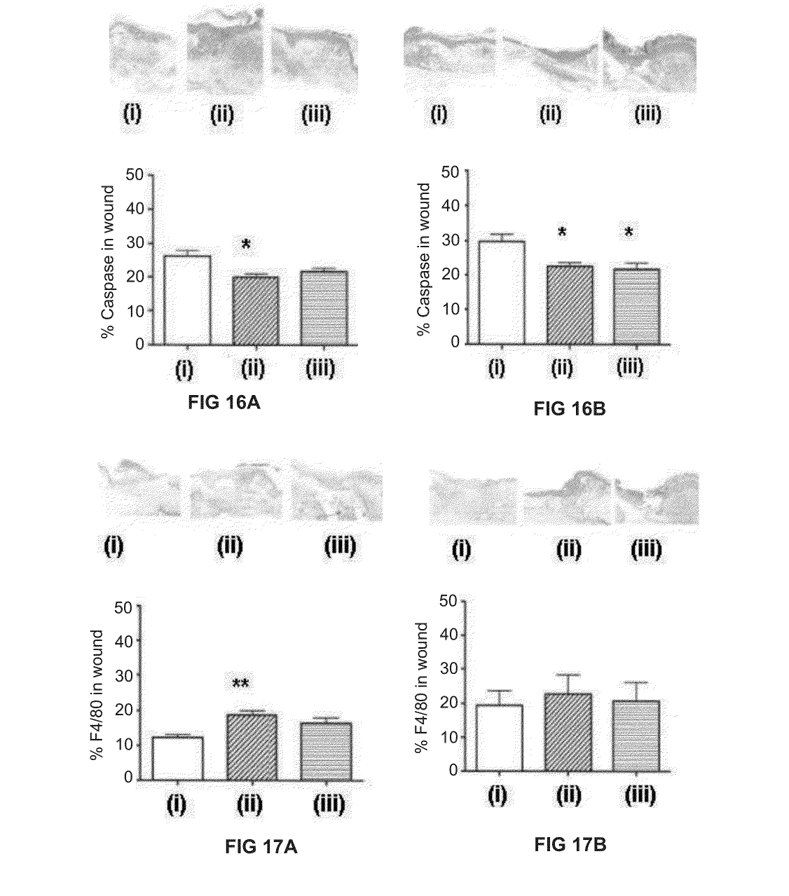

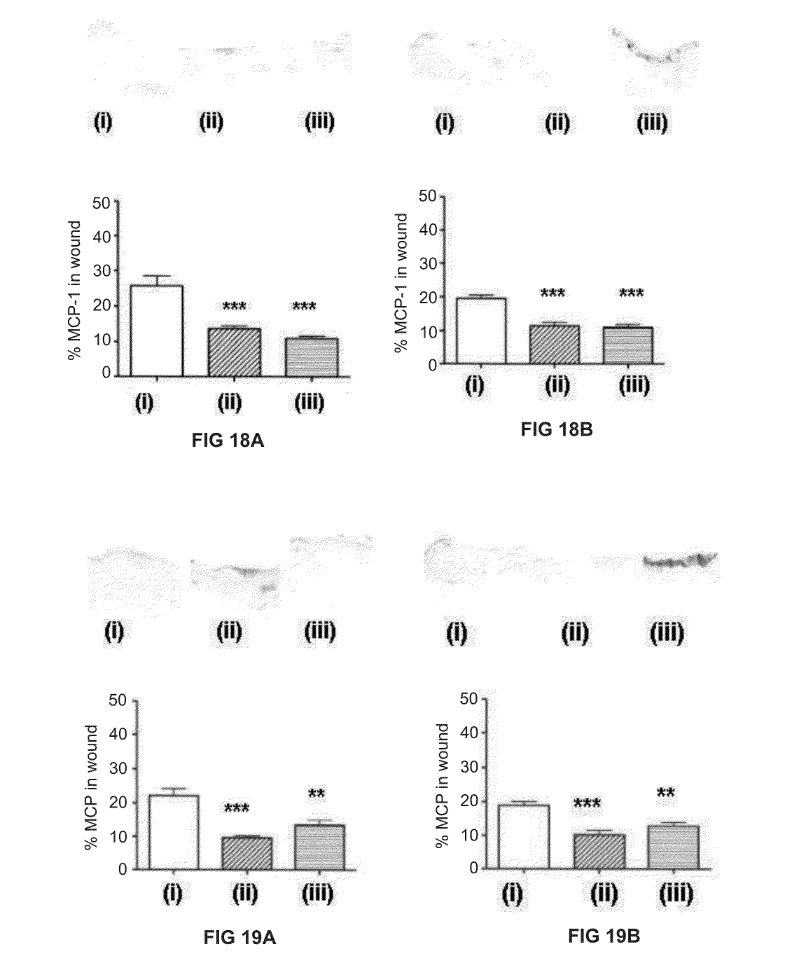

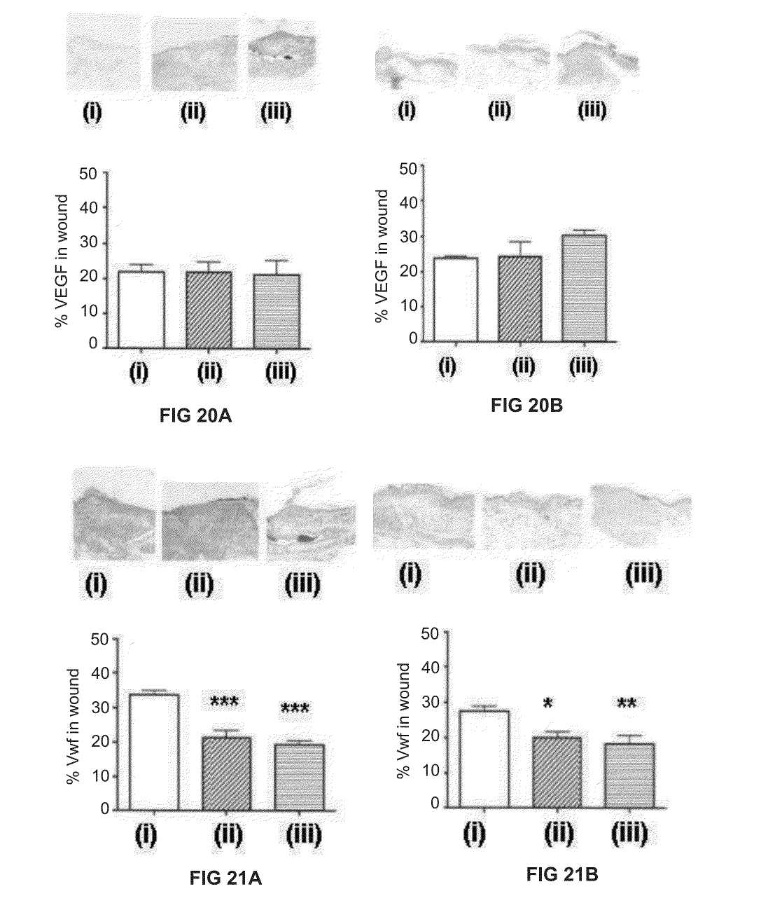

Tissue histology (FIGS. 12A to 21B) showed decreased levels of MPO (FIG. 10, p<0.001), MCP-1 (FIG. 9, p<0.001), IL-6 (FIG. 4, p<0.05) and vWF (FIG. 12A-B, p<0.01), and increased elastin levels (FIG. 15, p<0.01) in diabetic wounds treated with seleno-compounds compared to vehicle.

In the experimental testing according to the present invention, pairs of circular incision wounds on the backs of wildtype C57/BL6 and db−/db− mice (n=12 each) were treated daily with a seleno-compound according to the current invention or vehicle, topically for 10 days. Wound closure, vascular perfusion and tissue histology were assessed.

Wound closure in wildtype mice treated with a seleno-compound was 2-fold greater than controls at day 4 (32% vs 17.5%, p<0.05) and greater at day 10 (82% vs 65%, p<0.01).

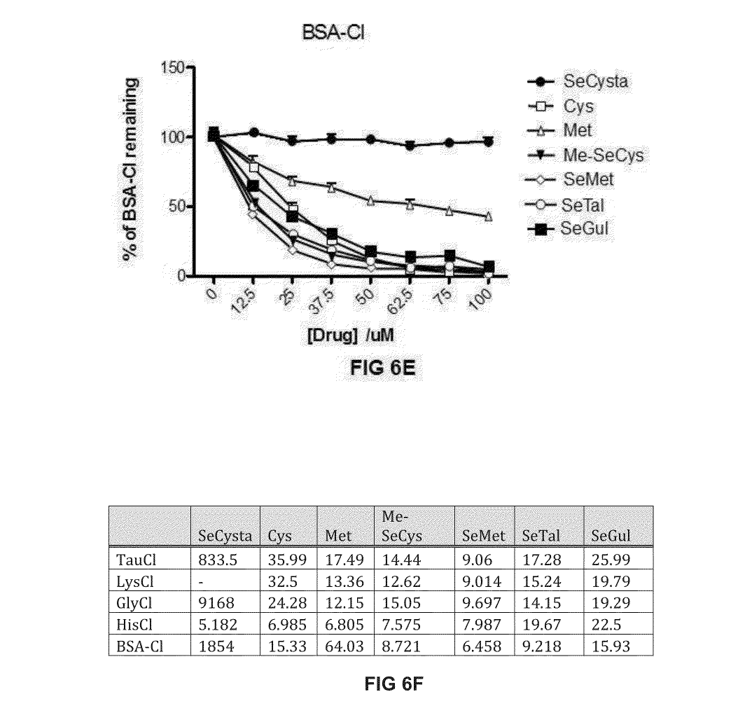

In some wounds, a reduction in IL-6 (FIG. 13A-B) is apparent, particularly in wounds associated with diabetic mice.

The observed results also include a reduction in apopotosis and an increase in elastin (FIG. 15A-B) during tissue repair.

Overall, the decrease in monocyte chemotactic activity, IL-6 expression and improvements in tissue elasticity and tensile strength indicates that treatment using the selenosugars of the present invention provides a surprising improvement in tissue repair.

While the seleno-compounds according to the present invention show a surprising improvement in the repair of tissue associated with non-diabetic wounds, they also are remarkably effective for the treatment of diabetic wounds.

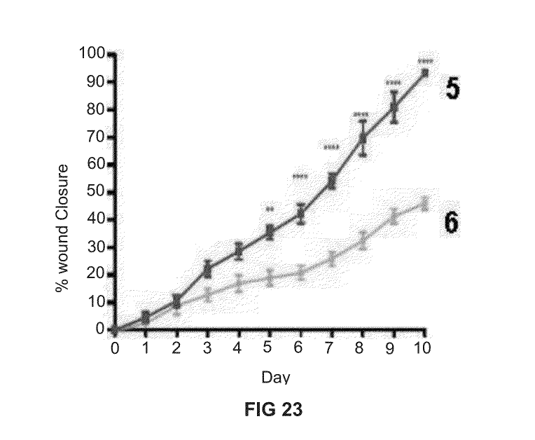

Test result data illustrated in FIG. 24A-B, show tissue repair during wound healing in wildtype mice following application of the seleno-compounds of formula II (8) and formula III (10).

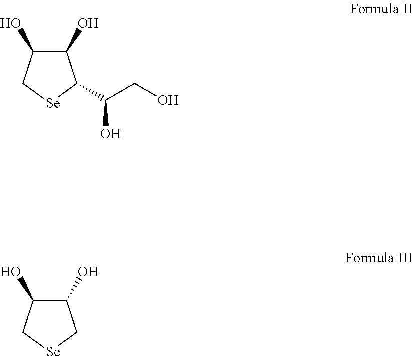 [SMILES: OC[C@@H](O)[C@@H]1[Se]C[C@@H](O)[C@H]1O ; O[C@@H]1C[Se]C[C@H]1O]

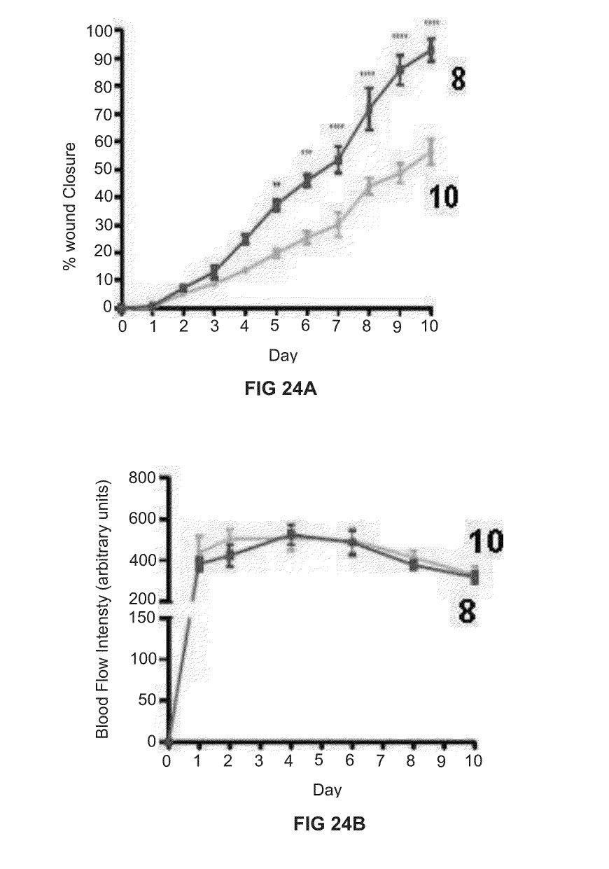

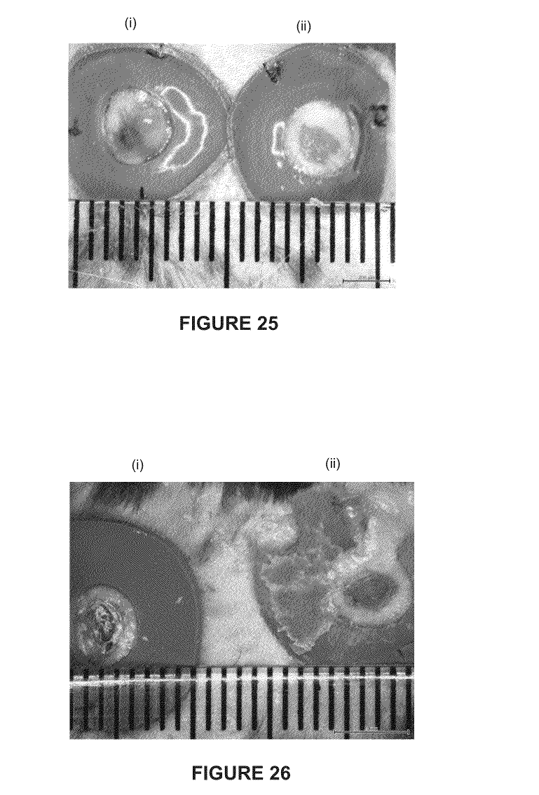

FIG. 25 illustrates reduced wound healing with observable pus formation after topical application of DL-trans-3,4,-Dihydroxy-L-selenolane (formula III), while the right image shows improved wound closure and healing by topical application of compound 1. FIG. 25 is a typical image representative of all of the mice in the study.

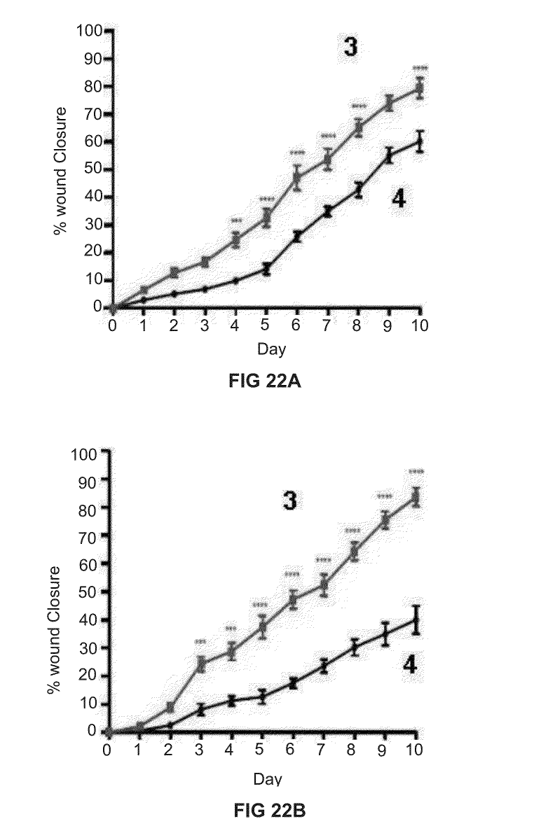

Thus the results illustrate the tissue repair and associated wound healing in diabetic patients (FIGS. 23 and 24A-B). In addition, the seleno-compound of formula III resulted in an inflammatory response during wound healing (FIG. 25).

1. A compound of formula (I): 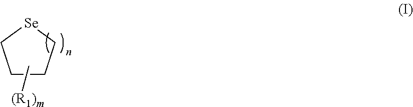
wherein n is 1, 2, or 3; m is 2, 3, 4, or 5; and each R₁ is independently (optionally substituted C1-C3 alkylene)p-OH, where p is 0 or 1, or a salt thereof.

2. A compound according to claim 1 or a salt thereof, selected from the following:

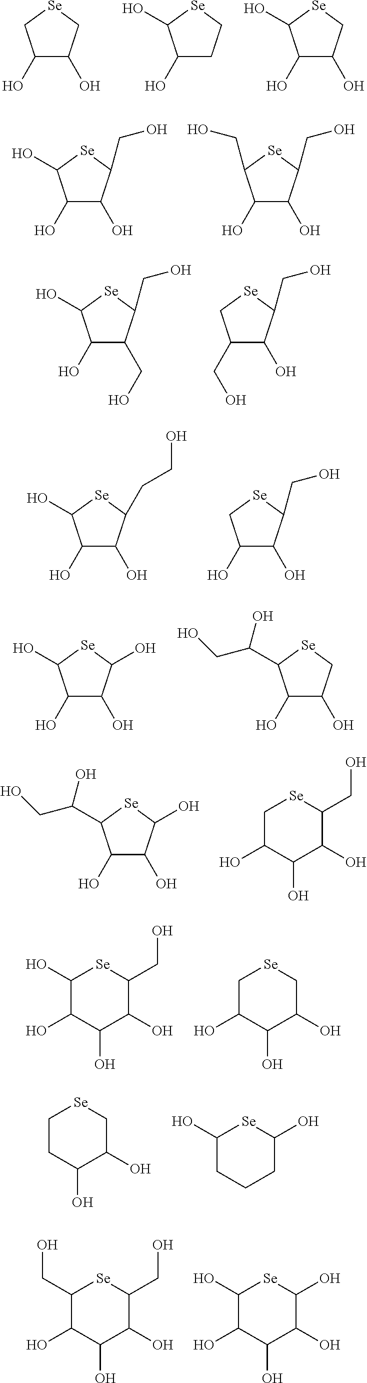

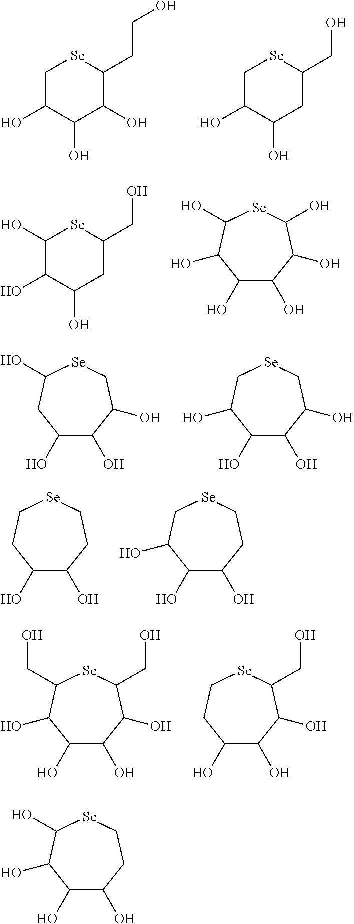

3. A compound according to claim I or a salt thereof, represented by:

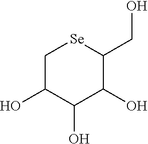 [SMILES: OCC1[Se]CC(O)C(O)C1O]

4. A compound according to claim 1 or a salt thereof, represented by:

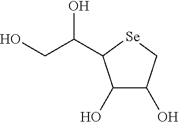 [SMILES: OCC(O)C1[Se]CC(O)C1O]

5. A compound according to claim 1 or a salt thereof, selected from one of following:
1,5-anhydro-5-seleno-L-gulitol;
1,5-anhydro-5-seleno-L-mannitol;
1,5-anhydro-5-seleno L-iditol;
1,5-anhydro-5-seleno-L-glucitol;
1,5-anhydro-5-seleno-L-galitol;
1,5-anhydro-5-seleno-L-talitol;
1,5-anhydro-5-seleno-L-allitol; and
1,5-anhydro-5-seleno-L-altritol.

6. A compound according to claim 1 or a salt thereof, selected from one of following:
1,5-anhydro-5-seleno-D-gulitol;
1,5-anhydro-5-seleno-D-mannitol;
1,5-anhydro-5-seleno-D-iditol;
1,5-anhydro-5-seleno-D-glucitol;
1,5-anhydro-5-seleno-D-galitol;
1,5-anhydro-5-seleno-D-talitol;
1,5-anhydro-5-seleno-D-allitol; and
1,5-anhydro-5-seleno-D-altritol.

7. A compound according to claim 1 or a salt thereof, selected from one of the following:
1,4-anhydro-4-seleno-L-gulitol;
1,4-anhydro; 4-seleno-L-mannitol;
1,4-anhydro-4-seleno-L-iditol;
1,4-anhydro-4-seleno-L-glucitol;
1,4-anhydro-4-seleno-L-galitol;
1,4-anhydro-4-seleno-L-talitol;
1,4-anhydro-4-seleno-L-allitol; and
1,4-anhydro-4-seleno-L-altritol.

8. A compound according to claim 1 or a salt thereof, selection from one of the following:
1,4-anhydro-4-seleno-D-gulitol;
1,4-anhydro-4-seleno-D-mannitol;
1,4-anhydro-4-seleno-D-iditol;
1,4-anhydro-4-seleno-D-glucitol;
1,4-anhydro-4-seleno-D-galitol;
1,4-anhydro-4-seleno-D-talitol;
1,4-anhydro-4-seleno-D-allitol; and
1,4-anhydro-4-seleno-D-altritol.

9. A pharmaceutical composition comprising a compound according to claim 1 or a pharmaceutically acceptable salt thereof, and a pharmaceutically acceptable carrier or diluent.

10. A method for the treatment of oxidative stress comprising the administration of a seleno-compound, or a pharmaceutically acceptable salt thereof, or a composition according to claim 1 to a subject in need thereof.

11. The method of claim 10 wherein the oxidative stress is associated with atherosclerosis.

12. The method of claim 10 wherein the oxidative stress is associated with cardiovascular disease.

13. A method of scavenging oxidants comprising the step of contacting a source of said oxidants with a seleno-compound if claim 1, or a pharmaceutically acceptable salt thereof, for a time and under suitable conditions.

14. A method of protecting against chloramine formation by HOCl, comprising the step of administering to a subject a compound or composition of claim 1.

15. A method of protecting a protein from HOCl-mediated oxidation comprising the step of contacting said protein with a compound or a composition of claim 1.

16. A method of protecting a protein from HOBr-mediated oxidation comprising the step of contacting said protein with a compound or a composition of claim 1.

17. A method of treating a disease or condition associated with increased levels of oxidants produced by MPO, the step of administering to a subject a compound or a composition of claim 1.

18. A method according to claim 16 wherein the disease or condition is atherosclerosis.

19. The method of claim 10 wherein the oxidative stress is associated with diabetes.

20. The method of claim 10 wherein the oxidative stress is associated with diabetic wound healing.
